(19) United States
(12) Patent Application Publication
(10) Pub. No.: US 2021/0010086 A1
(43) Pub. Date: Jan. 14, 2021

(54) MNK BIOMARKERS AND USES THEREOF

(71) Applicant: eFFECTOR Therapeutics, Inc., San Diego, CA (US)

(72) Inventors: Peggy A. Thompson, San Diego, CA (US); Wenqiong Joan Chen, San Diego, CA (US); James Appleman, San Diego, CA (US)

(21) Appl. No.: 16/881,193

(22) Filed: May 22, 2020

Related U.S. Application Data
(63) Continuation of application No. 15/389,822, filed on Dec. 23, 2016, now abandoned.
(60) Provisional application No. 62/273,875, filed on Dec. 31, 2015.

(51) Int. Cl. C12Q 1/6886; A61K 31/506; G01N 33/574

(52) U.S. Cl. CPC C12Q 1/6886; A61K 31/506; C12Q 2600/158; C12Q 2600/106; G01N 33/574

(57) ABSTRACT

The present disclosure relates to compositions and methods for identifying or diagnosing a human subject having or suspected of having a hyperproliferative disease and who would benefit from treatment with a MNK inhibitor.

### CROSS-REFERENCE TO RELATED APPLICATION

This application is a continuation of U.S. application Ser. No. 15/389,822 filed Dec. 23, 2016, which claims the benefit of U.S. Provisional Application No. 62/273,875 filed Dec. 31, 2015, which are hereby incorporated by reference in their entireties.

### BACKGROUND

Initiation of cap-dependent translation is thought to depend on the assembly of eukaryotic initiation factor 4F (eIF4F), an initiation factor complex including eIF4E, the scaffold protein eIF4G, and the RNA helicase eIF4A. The scaffold protein, eIF4G, contains binding sites for the cap binding eIF4E and the poly A tail protein (PABP) at the N-terminus, while the C-terminal domain contains docking sites for eIF3, eIF4A and Mnk1/2 (Pyronnet et al., EMBO J. 18:270, 1999; Imataka et al., EMBO J. 17: 7480, 1998; Lamphear et al., J. Biol. Chem. 270: 21975, 1995). eIF4G also recruits the 40S ribosomal subunit to the mRNA via its interaction with eIF3 and binds eIF4B, a protein that aids the

RNA-helicase function of eIF4A, thus facilitating the translation of mRNAs that contain structured 5′-untranslated terminal regions (UTRs). eIF4E is the key factor for the assembly of the eIF4F complex at the mRNA 5′-cap structure since eIF4E is the only protein of the complex that binds directly to the mRNA cap structure. Therefore, eIF4E is an important regulator of mRNA translation since the availability of eIF4E as part of the eIF4F complex is a limiting factor in controlling the rate of translation.

Regulation of eIF4E activity forms a node of convergence of the PI3K/Akt/mTOR and Erk/MAPK signaling pathways. The PI3K (phosphoinositide 3-kinase)/PTEN (phosphatase and tensin homologue deleted on chromosome ten)/Akt/mTOR (mammalian target of rapamycin) pathway is often involved in tumorgenesis, as well as sensitivity and resistance to cancer therapy. The Erk/MAPK signaling cascade is activated by growth factors and the p38 MAP kinase is part of a stress-activated pathway. The Mnk kinases can be activated by Erk and p38 MAPKs in response to various extracellular stimuli, and phosphorylate their major downstream effector, the cap binding eIF4E (Wang et al., J. Biol. Chem. 273: 9373, 1998). The Mnk kinases are also known to interact with the scaffold protein eIF4G (Shveygert et al., Mol. Cell Biol. 30:5160, 2010; Pyronnet et al., 1999; Scheper et al., Mol. Cell Biol. 21:743, 2001).

Mnk1 and Mnk2 are serine/threonine protein kinases that specifically phosphorylate serine 209 (Ser209) of eIF4E within the eIF4F complex. Mnk1 regulates eIF4E phosphorylation in response to external stimuli, while generally high basal Mnk2 activity, which is mostly unresponsive to external stimuli, accounts for the constitutive eIF4E phosphorylation levels (Waskiewicz et al., Mol. Cell Biol. 19:1871, 1999; Scheper et al., 2001). Mice with Mnkl, Mnk2 or both Mnk1 and Mnk2 inactivated are viable and phenotypically similar to wild type mice under unstressed conditions (Ueda et al., Mol. Cell Biol. 24:6539, 2004). In addition, mice with mutated eIF4E, in which Ser209 is replaced by alanine, show no eIF4E phosphorylation and significantly attenuated tumor growth (Furic et al., Proc. Nat'l. Acad. Sci. U.S.A. 107:14134, 2010). Phosphorylation of eIF4E is important for the translation of mRNAs containing 5′-UTRs with extensive secondary structure (Koromilas et al., EMBO J. 11:4153, 1992). Besides its ability to bind capped mRNA, nuclear eIF4E can interact with a 100 nt eIF4E-sensitive element (4E-SE) region in the 3′-UTRs of mRNAs and promote the nuclear export of the bound mRNA (Culjkovic et al., J. Cell Biol. 169:245, 2005). Each of Mnk1 and Mnk2 has alternatively spliced isoforms. Mnk1 has two alternatively spliced isoforms, Mnkla and Mnk1b, which differ at their carboxy-terminal end. The shorter Mnk1b isoform lacks exon 19, which results in a change in reading frame that introduces a premature stop codon (O′Loghlen et al., Exp. Cell Res. 299:343, 2004). Unlike Mnkla, Mnk1b also localizes to the nuclear compartment where it may regulate the phosphorylation of eIF4E and possibly other nuclear proteins (O'Loghlen et al., Biochim.

Biophys. Acta 1773:1416, 2007). Mnk1b exhibits higher basal activity as compared to Mnkla and lacks a MAPK domain (Goto et al., Biochem. J. 423:279, 2009). Mnk2 is also alternatively spliced into two isoforms, Mnk2a and Mnk2b. The two isoforms differ in their carboxy-terminal ends due to an alternative exon 13 (Scheper et al., Mol. Cell Biol. 23:5692, 2003). Mnk2b is shorter than Mnk2a, lacks a MAPK binding domain, exhibits low kinase activity towards eIF4E, and also localizes to the nucleus (Scheper et al., 2003).

The Mnk kinases are regulated by the p38 and Erk MAPK pathways, but their activity is also modulated by other MAPK-independent mechanisms. Mnk kinases can play an important role in controlling cap-dependent and cap-independent translation, participate in the pathophysiology of several malignant and inflammatory diseases and diminish responses to cancer therapeutics. Despite an increased understanding of Mnk structure and function, little progress has been made with regard to the discovery of pharmacological Mnk inhibitors and relatively few Mnk inhibitors have been reported: CGP052088 (Tschopp et al ., Mol Cell Biol Res Commun. 3(4):205-211, 2000); CGP57380 (Rowlett et al., Am J Physiol Gastrointest Liver Physiol. 294(2):G452-459, 2008); and cercosporamide (Konicek et al., Cancer Res. 71(5):1849-1857, 2011). More research efforts are needed to develop Mnk inhibitors to understand the variety of biological functions regulated or affected by Mnk kinases.

Accordingly, while advances have been made in this field there remains a significant need in the art for identifying biological functions regulated or altered by Mnk kinase activity, particularly with regard to Mnk's role in regulation of cancer pathways, as well as for associated composition and methods. The present disclosure meets such needs, and further provides other related advantages.

### BRIEF SUMMARY

In one aspect, the present disclosure provides a method of assessing whether a human subject having a hyperproliferative disease is likely to respond to treatment with a MNK inhibitor or of identifying a human subject as a candidate for treating a hyperproliferative disease with a MNK inhibitor.

In other aspects, the present disclosure provides a method for treating a hyperproliferative disease in a human subject, the method comprising administering an effective amount of a MNK inhibitor to a subject having or suspected of having a hyperproliferative disease when a sample obtained from the subject and prior to contacting the sample with a MNK inhibitor has a translational rate, translational efficiency, mRNA level or any combination thereof of one to about 100 genes as set forth in any of Tables 3-6, 9, 10 and 12 above or below a translational rate, translational efficiency, mRNA level or any combination thereof of one to about 100 genes as set forth in any of Tables 3-6, 9, 10 and 12 in the sample contacted with the MNK inhibitor. In certain embodiments, the translational rate, translational efficiency, mRNA level or any combination thereof of the one to about 100 genes as set forth in any of Tables 3-6, 9, 10 and 12 has at least about a log₂ fold change of 0.75 to about 2.0 (increase or decrease) as compared to a translational rate, translational efficiency, mRNA level or any combination thereof of the one to about 100 genes as set forth in any of Tables 3-6, 9, 10 and 12 in the sample contacted with the MNK inhibitor.

In further aspects, the present disclosure provides a method of maximizing therapeutic efficacy of a MNK inhibitor for a human subject having a hyperproliferative disease or monitoring response of a human subject having a hyperproliferative disease to treatment with a MNK inhibitor.

In still further aspects, the present disclosure provides a method of identifying a biomarker for determining responsiveness to a MNK inhibitor or for diagnosing a hyperproliferative disease in a human subject that would be responsive to a MNK inhibitor or of determining a prognosis of a human subject having a hyperproliferative disease if treated with a MNK inhibitor.

In yet further aspects, the present disclosure provides a kit for determining whether a human subject having a hyperproliferative disease may benefit from treatment with a MNK inhibitor.

### BRIEF DESCRIPTION OF THE DRAWINGS

FIG. 1 shows a western blot analysis of total cell lysates to assess protein levels of eIF4E and Cyclin D3 as well as phosphorylation levels of eIF4E after treating TMD8 cells with varying concentrations of Compound 107 for 3 or 48 hours.

FIGS. 2A and 2B show results of nascent protein synthesis analysis by measuring the uptake of L-azidohomoalanine (AHA) and polysome profiling of cells treated with a MNK inhibitor. (A) TMD8 cells were treated with DMSO or Compound 107 (300 nM and 10 μM) for 3 or 48 hours followed by labeling with AHA for 2 hours. The incorporated AHA was detected using western blot analysis and was normalized to a-actin. (B) Polysome profiles of TMD8 cells treated with DMSO (black) or 10 μM Compound 107 (red) for 3 or 48 hours. OD260, absorbance of light at 260 nm.

FIG. 3 shows a correlation plot of the Log₂ fold change in transcription (mRNA levels) versus translational rate (ribosome occupancy levels, RPF). TMD8 cells were treated with DMSO or Compound 107 (300 nM and 10 μM) for 3 or 48 hours. Data points in blue have p-values≤0.01 for changes in translational rate.

FIG. 4 shows a hierarchical cluster analysis of 215 genes identified by modulation of translational rate with Compound 107 treatment of TMD8 cells (10 μM, 48 hours) relative to DMSO treatment (Log₂ fold change≥0.75, p-value <0.01). RPF, ribosome protected fragments or translational rate; RNA, transcript levels. Color Key: red is upregulated; green is downregulated; scale (Log₂ fold change ±1.5).

FIG. 5 shows Gene Ontology biological process classification of MNK-sensitive genes identified by modulation of translational rate with Compound 107 treatment of TMD8 cells (10 μM, 48 hours) relative to DMSO treatment (Log₂ fold change >0.75, p-value≤0.01).

FIG. 6 show Western blot analysis of total cell lysates to assess protein levels of eIF4E and IRF7 as well as phosphorylation levels of eIF4E after treating TMD8 cells with varying concentrations of Compound 107 for 48 hours.

FIG. 7 shows the hierarchical cluster analysis of 51 genes identified by modulation of translational efficiency (TE) with Compound 107 treatment of TMD8 cells (300 nM and 10 μM for 3 hours) relative to control (Log₂ fold change≥1.0, p-value≤0.01). Color Key: red is upregulated; green is downregulated.; scale (Log₂ fold change±2.0).

### DETAILED DESCRIPTION

The instant disclosure provides compositions and methods for identifying human subjects and using biomarkers for preventing, ameliorating or treating a hyperproliferative disease that would be responsive to MNK inhibitors. For example, translational profiles may be used to determine translational efficiencies of one to about 100 genes as set forth in any of Tables 3-6, 9, 10 and 12 in a sample from the subject prior to contacting the sample with a MNK inhibitor, which can be compared to a control sample or a sample treated with the MNK inhibitor.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means ±20% of the indicated range, value, or structure, unless otherwise indicated. The term “consisting essentially of” limits the scope of a claim to the specified materials or steps, or to those that do not materially affect the basic and novel characteristics of the claimed invention. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

“Amino” refers to the —NH₂ substituent.

“Aminocarbonyl” refers to the —C(O)NH₂ substituent.

“Carboxyl” refers to the —CO₂H substituent.

“Carbonyl” refers to a —C(O)— or —C(═O)-group. Both notations are used interchangeably within the specification.

“Cyano” refers to the —C≡N substituent.

“Cyanoalkylene” refers to the -(alkylene)C≡N subsituent.

“Acetyl” refers to the —C(O)CH₃ substituent.

“Hydroxy” or “hydroxyl” refers to the —OH substituent.

“Hydroxyalkylene” refers to the -(alkylene)OH subsituent.

“Oxo” refers to a ═O substituent.

“Thio” or “thiol” refer to a —SH substituent.

“Alkyl” refers to a saturated, straight or branched hydrocarbon chain radical consisting solely of carbon and hydrogen atoms, having from one to twelve carbon atoms (C₁-C₁₂ alkyl), from one to eight carbon atoms (C₁-C₈ alkyl) or from one to six carbon atoms (C₁-C₆ alkyl), and which is attached to the rest of the molecule by a single bond. Exemplary alkyl groups include methyl, ethyl, n-propyl, 1-methylethyl (iso-propyl), n-butyl, n-pentyl, 1,1-dimethylethyl (t-butyl), 3-methylhexyl, 2-methylhexyl, and the like.

“Lower alkyl” has the same meaning as alkyl defined above but having from one to four carbon atoms (C₁-C₄ alkyl).

“Alkenyl” refers to an unsaturated alkyl group having at least one double bond and from two to twelve carbon atoms (C₂-C₁₂ alkenyl), from two to eight carbon atoms (C₂-C₈ alkenyl) or from two to six carbon atoms (C₂-C₆ alkenyl), and which is attached to the rest of the molecule by a single bond, e.g., ethenyl, propenyl, butenyl, pentenyl, hexenyl, and the like.

“Alkynyl” refers to an unsaturated alkyl group having at least one triple bond and from two to twelve carbon atoms (C₂-C₁₂ alkynyl), from two to ten carbon atoms (C₂-C₁₀ alkynyl) from two to eight carbon atoms (C₂-C₈ alkynyl) or from two to six carbon atoms (C₂-C₆ alkynyl), and which is attached to the rest of the molecule by a single bond, e.g., ethynyl, propynyl, butynyl, pentynyl, hexynyl, and the like.

“Alkylene” or “alkylene chain” refers to a straight or branched divalent hydrocarbon (alkyl) chain linking the rest of the molecule to a radical group, consisting solely of carbon and hydrogen, respectively. Alkylenes can have from one to twelve carbon atoms, e.g., methylene, ethylene, propylene, n-butylene, and the like. The alkylene chain is attached to the rest of the molecule through a single or double bond. The points of attachment of the alkylene chain to the rest of the molecule can be through one carbon or any two carbons within the chain. “Optionally substituted alkylene” refers to alkylene or substituted alkylene.

“Alkenylene” refers to divalent alkene. Examples of alkenylene include without limitation, ethenylene (—CH═CH—) and all stereoisomeric and conformational isomeric forms thereof “Substituted alkenylene” refers to divalent substituted alkene. “Optionally substituted alkenylene” refers to alkenylene or substituted alkenylene.

“Alkynylene” refers to divalent alkyne. Examples of alkynylene include without limitation, ethynylene, propynylene. “Substituted alkynylene” refers to divalent substituted alkyne.

“Alkoxy” refers to a radical of the formula —ORₐ where Rₐ is an alkyl having the indicated number of carbon atoms as defined above. Examples of alkoxy groups include without limitation —O-methyl (methoxy), —O-ethyl (ethoxy), —O-propyl (propoxy), —O-isopropyl (iso propoxy) and the like.

“Acyl” refers to a radical of the formula —C(O)Rₐ where Rₐ is an alkyl having the indicated number of carbon atoms. “Alkylaminyl” refers to a radical of the formula —NHRₐ or -NRₐRₐ where each Rₐ is, independently, an alkyl radical having the indicated number of carbon atoms as defined above.

“Cycloalkylaminyl” refers to a radical of the formula —NHRₐ where Rₐ is a cycloalkyl radical as defined herein.

“Alkylcarbonylaminyl” refers to a radical of the formula —NHC(O)Rₐ, where Rₐ is an alkyl radical having the indicated number of carbon atoms as defined herein.

“Cycloalkylcarbonylaminyl” refers to a radical of the formula —NHC(O)Rₐ, where Rₐ is a cycloalkyl radical as defined herein.

“Alkylaminocarbonyl” refers to a radical of the formula —C(O)NHRₐ or —C(O)NRₐRₐ, where each Rₐ is independently, an alkyl radical having the indicated number of carbon atoms as defined herein.

“Cyclolkylaminocarbonyl” refers to a radical of the formula —C(O)NHRₐ, where Rₐ is a cycloalkyl radical as defined herein.

“Aryl” refers to a hydrocarbon ring system radical comprising hydrogen, 6 to 18 carbon atoms and at least one aromatic ring. Exemplary aryls are hydrocarbon ring system radical comprising hydrogen and 6 to 9 carbon atoms and at least one aromatic ring; hydrocarbon ring system radical comprising hydrogen and 9 to 12 carbon atoms and at least one aromatic ring; hydrocarbon ring system radical comprising hydrogen and 12 to 15 carbon atoms and at least one aromatic ring; or hydrocarbon ring system radical comprising hydrogen and 15 to 18 carbon atoms and at least one aromatic ring. For purposes of this invention, the aryl radical may be a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. “Optionally substituted aryl” refers to an aryl group or a substituted aryl group.

“Arylene” denotes divalent aryl, and “substituted arylene” refers to divalent substituted aryl.

“Aralkyl” or “araalkylene” may be used interchangeably and refer to a radical of the formula —R_{b}-Rₑ where R_{b} is an alkylene chain as defined herein and R_{c} is one or more aryl radicals as defined herein, for example, benzyl, diphenylmethyl and the like.

“Cycloalkyl” refers to a stable non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, three to nine carbon atoms, three to eight carbon atoms, three to seven carbon atoms, three to six carbon atoms, three to five carbon atoms, a ring with four carbon atoms, or a ring with three carbon atoms. The cycloalkyl ring may be saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like.

“Cycloalkylalkylene” or “cycloalkylalkyl” may be used interchangeably and refer to a radical of the formula —R_{b}Rₑ where R_{b} is an alkylene chain as defined herein and Rₑ is a cycloalkyl radical as defined herein. In certain embodiments, R_{b} is further substituted with a cycloalkyl group, such that the cycloalkylalkylene comprises two cycloalkyl moieties. Cyclopropylalkylene and cyclobutylalkylene are exemplary cycloalkylalkylene groups, comprising at least one cyclopropyl or at least one cyclobutyl group, respectively.

“Fused” refers to any ring structure described herein which is fused to an existing ring structure in the compounds of the invention. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo (bromine), chloro (chlorine), fluoro (fluorine), or iodo (iodine).

“Haloalkyl” refers to an alkyl radical having the indicated number of carbon atoms, as defined herein, wherein one or more hydrogen atoms of the alkyl group are substituted with a halogen (halo radicals), as defined above. The halogen atoms can be the same or different. Exemplary haloalkyls are trifluoromethyl, difluoromethyl, trichloromethyl, 2,2,2-trifluoroethyl, 1,2-difluoroethyl, 3-bromo-2-fluoropropyl, 1,2-dibromoethyl, and the like.

“Heterocyclyl,” “heterocycle,” or “heterocyclic ring” refers to a stable 3- to 18-membered saturated or unsaturated radical which consists of two to twelve carbon atoms and from one to six heteroatoms, for example, one to five heteroatoms, one to four heteroatoms, one to three heteroatoms, or one to two heteroatoms selected from the group consisting of nitrogen, oxygen and sulfur. Exemplary heterocycles include without limitation stable 3-15 membered saturated or unsaturated radicals, stable 3-12 membered saturated or unsaturated radicals, stable 3-9 membered saturated or unsaturated radicals, stable 8-membered saturated or unsaturated radicals, stable 7-membered saturated or unsaturated radicals, stable 6-membered saturated or unsaturated radicals, or stable 5-membered saturated or unsaturated radicals.

Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of non-aromatic heterocyclyl radicals include, but are not limited to, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, thietanyl, trithianyl, tetrahydropyranyl, thiomorpholinyl, thiamorpholinyl, 1-oxo-thiomorpholinyl, and 1,1-dioxo-thiomorpholinyl. Heterocyclyls include heteroaryls as defined herein, and examples of aromatic heterocyclyls are listed in the definition of heteroaryls below.

“Heterocyclylalkyl” or “heterocyclylalkylene” refers to a radical of the formula —R_{b}R_{f} where R_{b} is an alkylene chain as defined herein and R_{f} is a heterocyclyl radical as defined above, and if the heterocyclyl is a nitrogen-containing heterocyclyl, the heterocyclyl may be attached to the alkyl radical at the nitrogen atom.

“Heteroaryl” or “heteroarylene” refers to a 5- to 14-membered ring system radical comprising hydrogen atoms, one to thirteen carbon atoms, one to six heteroatoms selected from the group consisting of nitrogen, oxygen and sulfur, and at least one aromatic ring.

For purposes of this invention, the heteroaryl radical may be a stable 5-12 membered ring, a stable 5-10 membered ring, a stable 5-9 membered ring, a stable 5-8 membered ring, a stable 5-7 membered ring, or a stable 6 membered ring that comprises at least 1 heteroatom, at least 2 heteroatoms, at least 3 heteroatoms, at least 4 heteroatoms, at least 5 heteroatoms or at least 6 heteroatoms. Heteroaryls may be a monocyclic, bicyclic, tricyclic or tetracyclic ring system, which may include fused or bridged ring systems; and the nitrogen,2 carbon or sulfur atoms in the heteroaryl radical may be optionally oxidized; the nitrogen atom may be optionally quaternized. The heteroatom may be a member of an aromatic or non-aromatic ring, provided at least one ring in the heteroaryl is aromatic.

Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (b enzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl).

“Heteroarylalkyl” or “heteroarylalkylene” refers to a radical of the formula —R_{b}R_{g} where R_{b} is an alkylene chain as defined above and R_{g} is a heteroaryl radical as defined above.

“Thioalkyl” refers to a radical of the formula -SRₐ where Rₐ is an alkyl radical as defined above containing one to twelve carbon atoms, at least 1-10 carbon atoms, at least 1-8 carbon atoms, at least 1-6 carbon atoms, or at least 1-4 carbon atoms.

“Heterocyclylaminyl” refers to a radical of the formula —NHR_{f} where R_{f} is a heterocyclyl radical as defined above.

“Thione” refers to a =S group attached to a carbon atom of a saturated or unsaturated (C₃-C₈)cyclic or a (C₁-C₈)acyclic moiety.

“Sulfoxide” refers to a —S(O)— group in which the sulfur atom is covalently attached to two carbon atoms.

“Sulfone” refers to a —S(O)₂— group in which a hexavalent sulfur is attached to each of the two oxygen atoms through double bonds and is further attached to two carbon atoms through single covalent bonds.

The term “oxime” refers to a —C(Rₐ)═N—ORₐ radical where Rₐ is hydrogen, lower alkyl, an alkylene or arylene group as defined above.

The compound of the invention can exist in various isomeric forms, as well as in one or more tautomeric forms, including both single tautomers and mixtures of tautomers. The term “isomer” is intended to encompass all isomeric forms of a compound of this invention, including tautomeric forms of the compound.

Some compounds described here can have asymmetric centers and therefore exist in different enantiomeric and diastereomeric forms. A compound of the invention can be in the form of an optical isomer or a diastereomer. Accordingly, the invention encompasses compounds of the invention and their uses as described herein in the form of their optical isomers, diastereoisomers and mixtures thereof, including a racemic mixture. Optical isomers of the compounds of the invention can be obtained by known techniques such as asymmetric synthesis, chiral chromatography, or via chemical separation of stereoisomers through the employment of optically active resolving agents.

Unless otherwise indicated, “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

In this description, a “pharmaceutically acceptable salt” is a pharmaceutically acceptable, organic or inorganic acid or base salt of a compound of the invention. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure.

As used herein, the term “derivative” refers to a modification of a compound by chemical or biological means, with or without an enzyme, which modified compound is structurally similar to a parent compound and (actually or theoretically) derivable from that parent compound. Generally, a “derivative” differs from an “analog” in that a parent compound may be the starting material to generate a “derivative,” whereas the parent compound may not necessarily be used as the starting material to generate an “analog.” A derivative may have different chemical, biological or physical properties from the parent compound, such as being more hydrophilic or having altered reactivity as compared to the parent compound. Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). Other exemplary derivatizations include glycosylation, alkylation, acylation, acetylation, ubiqutination, esterification, and amidation.

The term “derivative” also refers to all solvates, for example, hydrates or adducts (e.g., adducts with alcohols), active metabolites, and salts of a parent compound. The type of salt depends on the nature of the moieties within the compound. For example, acidic groups, such as carboxylic acid groups, can form alkali metal salts or alkaline earth metal salts (e.g., sodium salts, potassium salts, magnesium salts, calcium salts, and also salts with physiologically tolerable quaternary ammonium ions and acid addition salts with ammonia and physiologically tolerable organic amines such as, for example, triethylamine, ethanolamine or tris-(2-hydroxyethyl)amine). Basic groups can form acid addition salts with, for example, inorganic acids such as hydrochloric acid, sulfuric acid or phosphoric acid, or with organic carboxylic acids or sulfonic acids such as acetic acid, citric acid, lactic acid, benzoic acid, maleic acid, fumaric acid, tartaric acid, methanesulfonic acid or p-toluenesulfonic acid. Compounds that simultaneously contain a basic group and an acidic group, for example, a carboxyl group in addition to basic nitrogen atoms, can be present as zwitterions. Salts can be obtained by customary methods known to those skilled in the art, for example, by combining a compound with an inorganic or organic acid or base in a solvent or diluent, or from other salts by cation exchange or anion exchange.

The term “prodrug” refers to a precursor of a drug, a compound which upon administration to a patient, must undergo chemical conversion by metabolic processes before becoming an active pharmacological agent. Exemplary prodrugs of compounds in accordance with Formula I are esters, acetamides, and amides.

As used herein, the term “MNK,” also known as “mitogen-activated protein kinase (MAPK)-interacting serine/threonine kinase” or “MKNK” refers to a kinase that is phosphorylated by the p42 MAP kinases ERK1 and ERK2 and the p38-MAP kinases, triggered in response to growth factors, phorbol esters, and oncogenes such as Ras and Mos, and by stress signaling molecules and cytokines. MNK also refers to a kinase that is phosphorylated by additional MAP kinase(s) affected by interleukin-1 receptor-associated kinase 2 (IRAK2) and IRAK4, which are protein kinases involved in signaling innate immune responses through toll-like receptors (e.g., TLR₇) (see, e.g., Wan et al., J. Biol. Chem. 284: 10367, 2009). Phosphorylation of MNK proteins stimulates their kinase activity toward eukaryotic initiation factor 4E (eIF4E), which in turn regulates cap-dependent protein translation initiation, as well as regulate engagement of other effector elements, including hnRNPA1 and PSF (PTB (polypyrimidine tract binding protein) associated splicing factor). For example, proteins that bind the regulatory AU-rich elements (AREs) of the 3′-UTR of certain mRNAs (e.g., cytokines) are phosphorylated by MNK. Thus, MNK phosphorylation of proteins can alter the ability of these proteins to bind the 5′- or 3′-UTRs of eukaryotic mRNAs. In particular, reduced MNK mediated phosphorylation of hnRNPA1 decreases its binding to cytokine-ARE (see, e.g., Buxade et al., Immunity 23:177, 2005; Joshi and Platanias, Biomol. Concepts 3:127, 2012). MNK is encoded by two different genes, MNK1 and MNK2, which are both subject to alternative splicing. MNK1a and MNK2a represent full length transcripts, while MNK1b and MNK2b are splice variants that lack a MAPK binding domain. Therefore, MNK may refer to MNK1 or variants thereof (such as MNK1a or MNK1b), MNK2 or variants thereof (such as MNK2a or MNK2b), or combinations thereof In particular embodiments, MNK refers to human MNK.

The term “inhibit” or “inhibitor” refers to an alteration, interference, reduction, down regulation, blocking, abrogation or degradation, directly or indirectly, in the expression, amount or activity of a target or signaling pathway relative to (1) a control, endogenous or reference target or pathway, or (2) the absence of a target or pathway, wherein the alteration, interference, reduction, down regulation, blocking, abrogation or degradation is statistically, biologically, or clinically significant.

For example, a “MNK inhibitor” may block, inactivate, reduce or minimize MNK activity (e.g., kinase activity or translational effects), or reduce activity by promoting degradation of MNK, by about 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or more as compared to untreated MNK. In certain embodiments, a MNK inhibitor blocks, inactivates, reduces or minimizes the ability of MNK to phosphorylate eIF4E, hnRNPA1, PSF or combinations thereof. In further embodiments, a MNK inhibitor reduces or minimizes the expression of an immunosuppressive signal component, such as a ligand on a tumor cell or APC (e.g., PD-L1), a receptor on a T cell (e.g., PD-1, LAG3), or an immunosuppressive cytokine produced by such cells (e.g., IL-10, IL-4, IL-1RA, IL-35). Non-limiting examples of inhibitors include small molecules, antisense molecules, ribozymes, RNAi molecules, or the like.

In certain embodiments, a MNK inhibitor has specificity or is specific for MNK. As used herein, a “specific MNK inhibitor” has at least 25-fold less activity against the rest of a host cell kinome, which is the subset of genes that code for protein kinases in the genome of an organism or cell of interest (e.g., human). In further embodiments, a specific MNK inhibitor is a small molecule and has at least 50-fold less activity against the rest of the serine/threonine kinome of a cell, such as a human cell. In further embodiments, a specific MNK inhibitor has at least 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold less, or even less activity against other kinome enzymes. In still further embodiments, a specific MNK inhibitor blocks, inactivates, reduces or minimizes the ability of MNK1a, MNK1b, MNK2a, MNK2b, or any combination thereof to phosphorylate eIF4E, hnRNPA1, PSF or combinations thereof. Assays for detecting kinase activity in the presence or absence of inhibitors are well known in the art and can be used to show a particular MNK inhibitor is a specific MNK inhibitor, such as the assay taught by Karaman et al. (Nat. Biotechnol. 26:127, 2007).

As used herein, the term “translational profile” refers to the amount of protein made from translation of mRNA (i.e., translational level) for each gene in a given set of genes in a biological sample, collectively representing a set of individual translational rate values, translational efficiency values, or both translational rate and translational efficiency values for each of one or more genes in a given set of genes. In some embodiments, a translational profile comprises translational levels for a plurality of genes in a biological sample (e.g., cells), e.g., for at least about 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000 genes or more, or for at least about 1%, 2%, 3%, 4%, 5%, 6%, 7%, 8%, 9%, 10%, 15%, 20%, 25%, 50% or more of all genes in the sample. In some embodiments, a translational profile comprises a genome-wide measurement of translational rate, translational efficiency or both in a biological sample. In certain embodiments, a translational profile refers to a quantitative measure of the amount of mRNA associated with one or more ribosomes for each gene (i.e., translational rate, efficiency or both) in a given set of genes in a biological sample, wherein the amount of ribosome-associated mRNA correlates to the amount of protein that is translated (i.e., translational level).

As used herein, “translation rate” or “rate of translation” or “translational rate” refers to the total count of ribosome engagement, association or occupancy of mRNA for a particular gene as compared to the total count of ribosome engagement, association or occupancy of mRNA for at least one other gene or set of genes, wherein the count of total ribosomal occupancy correlates to the level of protein synthesis. Examination of translation rate across individual genes may be quantitative or qualitative, which will reveal differences in translation. In certain embodiments, translational rate provides a measure of protein synthesis for one or more genes, a plurality of genes, or across an entire genome. In particular embodiments, a translation rate is the amount of mRNA fragments protected by ribosomes for a particular gene relative to the amount of mRNA fragments protected by ribosomes for one or more other genes or groups of genes. For example, the mRNA fragments protected by ribosomes may correspond to a portion of the 5′-untranslated region, a portion of the coding region, a portion of a splice variant coding region, or combinations thereof In further embodiments, the translation rate is a measure of one, a plurality or all mRNA variants of a particular gene. Translation rates can be established for one or more selected genes or groups of genes within a single composition (e.g., biological sample), between different compositions, or between a composition that has been split into at least two portions and each portion exposed to different conditions.

As used herein, “mRNA level” refers to the amount, abundance, or concentration of mRNA or portions thereof for a particular gene in a composition (e.g., biological sample).

In certain embodiments, mRNA level refers to a count of one mRNA, a plurality of mRNA or all mRNA forms or fragments for a particular gene, including pre-mRNA, mature mRNA, or splice variants thereof In particular embodiments, an mRNA level for one or more genes or groups of genes corresponds to counts of unique mRNA sequences or portions thereof for a particular gene that map to a 5′-untranslated region, a coding region, a splice variant coding region, or any combination thereof.

As used herein, “translation efficiency” or “translational efficiency” refers to the ratio of the translation rate for a particular gene to the mRNA level for a particular gene in a given set of genes. For example, gene X may produce an equal abundance of mRNA (i.e., same or similar mRNA level) in normal and diseased tissue, but the amount of protein X produced may be greater in diseased tissue as compared to normal tissue. In this situation, the message for gene X is more efficiently translated in diseased tissue than in normal tissue (i.e., an increased translation rate without an increase in mRNA level). In another example, gene Y may produce half the mRNA level in normal tissue as compared to diseased tissue, and the amount of protein Y produced in normal tissue is half the amount of protein Y produced in diseased tissue. In this second situation, the message for gene Y is translated equally efficiently in normal and diseased tissue (i.e., a change in translation rate in diseased tissue that is proportional to the increase in mRNA level and, therefore, the translational efficiency is unchanged). In other words, the expression of gene X is altered at the translational level, while gene Y is altered at the transcriptional level. In certain situations, both the amount of mRNA and protein may change such that mRNA abundance (transcription), translation rate, translation efficiency, or a combination thereof is altered relative to a particular reference or standard.

In certain embodiments, translational efficiency may be standardized by measuring a ratio of ribosome-associated mRNA read density (i.e., translation level) to mRNA abundance read density (i.e., transcription level) for a particular gene. As used herein, “read density” is a measure of mRNA abundance and protein synthesis (e.g., ribosome profiling reads) for a particular gene, wherein at least 5, 10, 15, 20, 25, 50, 100, 150, 175, 200, 225, 250, 300 reads or more per unique mRNA or portion thereof is performed in relevant samples to obtain single-gene quantification for one or more treatment conditions. In certain embodiments, translational efficiency is scaled to standardize or normalize the translational efficiency of a median gene to 1.0 after excluding regulated genes (e.g., log2 fold-change ±1.5 after normalizing for the all-gene median), which corrects for differences in the absolute number of sequencing reads obtained for different libraries. In further embodiments, changes in protein synthesis, mRNA abundance and translational efficiency are similarly computed as the ratio of read densities between different samples and normalized to give a median gene a ratio of 1.0, normalized to the mean, normalized to the mean or median of log values, or the like.

As used herein, “gene signature” refers to a plurality of genes that exhibit a generally coherent, systematic, coordinated, unified, collective, congruent, or signature expression pattern or translation efficiency. In certain embodiments, a gene signature is (a) a plurality of genes that together comprise at least a detectable or identifiable portion of a biological pathway affected by a MNK inhibitor (e.g., 2, 3, 4, 5, or more genes; a hyperproliferative disease gene signature can comprise up to 10, 11, 12, 13, 14, 15, 16, 17, 18, 129, or 20 genes from a particular pathway, such as genes regulated by the eIF4F complex or component thereof, such as eIF4A or eIF4E), (b) a complete set of genes associated with a biological pathway affected by a MNK inhibitor, or (c) a cluster or grouping of independent genes having a recognized pattern of expression associated with being contacted with a MNK inhibitor. One or more genes from a particular gene signature may be part of a different gene signature (e.g., a cell migration pathway may share a gene with a cell adhesion pathway)—that is, gene signatures may intersect or overlap but each signature can still be independently defined by its unique translation profile.

The term “modulate” or “modulator,” as used with reference to altering an activity of a target gene or signaling pathway, refers to increasing (e.g., activating, facilitating, enhancing, agonizing, sensitizing, potentiating, or up regulating) or decreasing (e.g., preventing, blocking, inactivating, delaying activation, desensitizing, antagonizing, attenuating, or down regulating) the activity of the target gene or signaling pathway. In certain embodiments, a modulator alters a translational profile at the translational level (i.e., increases or decreases translation rate, translation efficiency or both, as described herein), at the transcriptional level, or both.

As used herein, a modulator or agent that “specifically binds” or is “specific for” a target refers to an association or union of a modulator or agent (e.g., siRNA, chemical compound) to a target molecule (e.g., a nucleic acid molecule encoding a target, a target product encoded by a nucleic acid molecule, or a target activity), which may be a covalent or non-covalent association, while not significantly associating or uniting with any other molecules or components in a cell, tissue, biological sample, or subject. A modulator or agent specific for a target (e.g., translation machinery component, such as eIF4E; translation machinery regulator, such as eIF2AK1, eIF2AK2, eIF2AK3, eIF2AK4) includes analogs and derivatives thereof In certain embodiments, a modulator specific for a translation machinery component (e.g., eIF4E) or translation machinery regulator (e.g., eIF2AK1) is a siRNA molecule.

In some embodiments, an agent that modulates translation in a hyperproliferative disease is identified as suitable for use when one or more genes of one or more biological pathways, gene signatures or combinations thereof are differentially translated by at least 1.5-fold (e.g., at least 1.5-fold, at least 2-fold, at least 2.5-fold, at least 3-fold, at least 3.5-fold, at least 4-fold, at least 4.5-fold, at least 5-fold, at least 6-fold, at least 7-fold, at least 8-fold, at least 9-fold, at least 10-fold or more) in a first translational profile (e.g., treated hyperproliferative disease sample, control sample or normal sample) as compared to a second translational profile (e.g., untreated disease or control sample). In some embodiments, an agent that modulates translation in a hyperproliferative disease is identified as suitable for use when the translational rate, translational efficiency, mRNA level or any combination thereof for one or more genes of one or more biological pathways, gene signatures or combinations thereof are increased or decreased by at least 1.5-fold (e.g., at least 1.5-fold, at least 2-fold, at least 2.5-fold, at least 3-fold, at least 3.5-fold, at least 4-fold, at least 4.5-fold, at least 5-fold, at least 6-fold, at least 7-fold, at least 8-fold, at least 9-fold, at least 10-fold or more) in a first translational profile as compared to a second translational profile.

A “biological sample” includes blood and blood fractions or products (e.g., serum, plasma, platelets, red blood cells, or the like); sputum or saliva; kidney, lung, liver, heart, brain, nervous tissue, thyroid, eye, skeletal muscle, cartilage, or bone tissue; cultured cells, e.g., primary cultures, explants, and transformed cells, stem cells, stool, urine, etc. Such biological samples (e.g., disease samples or normal samples) also include sections of tissues, such as a biopsy or autopsy sample, frozen sections taken for histologic purposes, or cells or other biological material used to model disease or to be representative of a pathogenic state. In certain embodiments, a biological sample is obtained from a “subject,” e.g., a eukaryotic organism, most preferably a mammal such as a primate, e.g., chimpanzee or human; cow; dog; cat; rodent, e.g., guinea pig, rat, or mouse; rabbit; bird; reptile; or fish.

As used herein, the term “normalize” or “normalizing” or “normalization” refers to adjusting the translational rate, translational efficiency, mRNA level or any combination thereof of one or more genes in a biological sample from a subject (e.g., a disease sample from one or more subjects, tissues or organs) to a level that is more similar, closer to, or comparable to the translational rate, translational efficiency, mRNA level or any combination thereof of those same one or more genes in a control sample (e.g., a non-diseased or normal sample from the same or different subject, tissue or organ). In certain embodiments, normalization refers to modulation of one or more translational regulators or translational system components to adjust or shift the translational rate, efficiency or both of one or more genes in a biological sample (e.g., diseased, abnormal or other biologically altered condition) to a translational efficiency that is more similar, closer to or comparable to the translational efficiency of those one or more genes in a non-diseased or normal control sample. In some embodiments, normalization is evaluated by determining a translational rate, translational efficiency, mRNA level or any combination thereof of one or more genes in a biological sample (e.g., disease sample) from a subject before and after an agent (e.g., therapeutic or known active agent) is administered to the subject and comparing the translational rate, translational efficiency, mRNA level or any combination thereof before and after administration to the translational rate, translational efficiency, mRNA level or any combination thereof from a control sample in the absence or presence of the agent. Exemplary methods of evaluating normalization of a translational profile associated with a disease or disorder includes observing a shift in a gene signature or evaluating a translational profile shift due to a therapeutic intervention in a hyperproliferative condition, disease or disorder.

As used herein, the phrase “differentially translated” refers to a change or difference (e.g., increase, decrease or a combination thereof) in translation rate, translation efficiency, or both of one gene, a plurality of genes, a set of genes of interest, one or more gene clusters, or one or more gene signatures under a particular condition as compared to the translation rate, translation efficiency, or both of the same gene, plurality of genes, set of genes of interest, gene clusters, or gene signatures under a different condition, which is observed as a difference in expression pattern. For example, a translational profile of a diseased cell may reveal that one or more genes have higher translation rates, higher translation efficiencies, or both (e.g., higher ribosome engagement of mRNA or higher protein abundance) than observed in a control or normal cell. Another exemplary translational profile of a diseased cell may reveal that one or more genes have lower translation rates, lower translation efficiencies, or both (e. g. , lower ribosome engagement of mRNA or lower protein abundance) than observed in a control or normal cell. In still another example, a translational profile of a diseased cell may reveal that one or more genes have higher translation rates, one or more genes have higher translation efficiencies, one or more genes have lower translation rates, one or more genes have lower translation efficiencies, or any combination thereof than observed in a control or normal cell. In some embodiments, one or more gene signatures, gene clusters or sets of genes of interest are differentially translated in a first translational profile as compared to one or more other translational profiles. In further embodiments, one or more genes, gene signatures, gene clusters or sets of genes of interest in a first translational profile show at least a 1.5-fold translation differential or at least a 1.0 log2 change (i.e., increase or decrease) as compared to the same one or more genes in at least one other different (e.g., second, third, etc.) translational profile.

In some embodiments, two or more translational profiles are generated and compared to each other to determine the differences (i.e., increases and/or decreases in translational rate, translational efficiency, mRNA level or any combination thereof) for each gene in a given set of genes between the two or more translational profiles. The comparison between the two or more translational profiles is referred to as the “differential translational profile.” In certain embodiments, a differential translational profile comprises one or more genes, gene clusters, or gene signatures (e.g., a hyperproliferative disease-associated pathway), or combinations thereof.

In certain embodiments, differential translation between genes or translational profiles may involve or result in a biological (e.g., phenotypic, physiological, clinical, therapeutic, prophylactic) benefit. For example, when identifying a therapeutic, validating a target, or treating a subject having a hyperproliferative disease, a “biological benefit” means that the effect on translation rate, translation efficiency or both, or the effect on the translation rate, translation efficiency or both of one or more genes of a translational profile allows for intervention or management of the hyperproliferative disease of a subject (e.g., a human or non-human mammal, such as a primate, horse, dog, mouse, rat). In general, one or more differential translations or differential translation profiles indicate that a “biological benefit” will be in the form, for example, of an improved clinical outcome; lessening or alleviation of symptoms associated with a hyperproliferative disease; decreased occurrence of symptoms; improved quality of life; longer disease-free status; diminishment of extent of hyperproliferative disease; stabilization of a hyperproliferative disease; delay of hyperproliferative disease progression; remission; survival; or prolonging survival. In certain embodiments, a biological benefit comprises normalization of a differential translation profile, or comprises a shift in translational profile to one closer to or comparable to a translational profile induced by a known active compound or therapeutic, or comprises inducing, stimulating or promoting a desired phenotype or outcome (e.g., reversal of transformation, induction of a quiescent state, apoptosis, necrosis, cytotoxicity), or reducing, inhibiting or preventing an undesired phenotype or outcome (e.g., activation, transformation, proliferation, migration).

In some embodiments, less than about 20% of the genes in the genome are differentially translated by at least 1.5-fold in a first translational profile as compared to a second translational profile. In some embodiments, less than about 5% of the genes in the genome are differentially translated by at least 2-fold or at least 3-fold in a first translational profile as compared to a second translational profile. In some embodiments, less than about 1% of the genes in the genome are differentially translated by at least 4-fold or at least 5-fold in a first translational profile as compared to a second translational profile.

As described herein, differentially translated genes between first and second translational profiles under a first condition may exhibit translational profiles “closer to” each other (i.e., identified through a series of pair-wise comparisons to confirm a similarity of pattern) under one or more different conditions (e.g., differentially translated genes between a normal sample and a hyperproliferative disease sample may have a more similar translational profile when the normal sample is compared to a hyperproliferative disease sample contacted with a MNK inhibitor). In certain embodiments, a test translational profile is “closer to” a reference translational profile when at least 99%, 95%, 90%, 80%, 70%, 60%, 50%, 25%, or 10% of a selected portion of differentially translated genes, a majority of differentially translated genes, or all differentially translated genes show a translational profile within 75%, 70%, 65%, 60%, 55%, 50%, 45%, 40%, 35%, 30%, or 25%, respectively, of their corresponding genes in the reference translational profile. In further embodiments, a selected portion of differentially translated genes, a majority of differentially translated genes, or all differentially translated genes from an experimental translational profile have a translational profile “closer to” the translational profile of the same genes in a reference translational profile when the amount of protein translated in the experimental and reference translational profiles are within about 3.0 log₂, 2.5 log₂, 2.0 log₂, 1.5 log₂, 1.1 log₂, 0.5 log₂, 0.2 log₂ or closer. In still further embodiments, a selected portion of differentially translated genes, a majority of differentially translated genes, or all differentially translated genes from an experimental translational profile have a translational profile “closer to” the translational profile of the same genes in a reference translational profile when the amount of protein translated in the experimental and reference translational profiles differs by no more than about 50%, 45%, 40%, 35%, 30%, 25%, 20%, 15%, 10%, 5%, 1% or less.

In some embodiments, an experimental differential expression profile as compared to a reference differential expression profile of interest has at least a 1.0 log₂ change in translational rate, translational efficiency, mRNA level or any combination thereof for at least 0.05%, at least 0.1%, at least 0.25%, at least 0.5%, at least 1%, at least 2%, at least 3%, at least 4%, at least 5%, at least 6%, at least 7%, at least 8%, at least 9%, at least 10%, at least 15%, at least 20%, at least 30%, at least 40%, at least 50%, at least 60%, at least 70%, at least 80%, or at least 90% or more of a set of selected differentially translated genes or for the entire set of selected differentially translated genes. In some embodiments, an experimental differential profile as compared to a reference differential expression profile of interest has at least a 2 log2 change in translational rate, translational efficiency, mRNA level or any combination thereof for at least 0.05%, at least 0.1%, at least 0.25%, at least 0.5%, at least 1%, at least 5%, at least 10%, at least 15%, at least 20%, at least 30%, at least 40%, at least 50%, at least 60%, at least 70%, at least 80%, or at least 90% or more of a set of selected differentially translated genes or for the entire set of differentially translated or transcribed genes. In some embodiments, an experimental differential expression profile as compared to a reference differential expression profile of interest has at least a 3 loge change in translational rate, translational efficiency, mRNA level or any combination thereof for at least 0.05%, at least 0.1%, at least 0.25%, at least 0.5%, at least 1%, at least 5%, at least 10%, at least 15%, at least 20%, at least 30%, at least 40%, at least 50%, at least 60%, at least 70%, at least 80%, or at least 90% or more of a set of selected differentially expressed genes or for the entire set of selected differentially expressed genes. In some embodiments, an experimental differential expression profile as compared to a reference differential expression profile of interest has at least a 4 log2 change in translational levels for at least 0.05%, at least 0.1%, at least 0.25%, at least 0.5%, at least 1%, at least 5%, at least 10%, at least 15%, at least 20%, at least 30%, at least 40%, at least 50%, at least 60%, at least 70%, at least 80%, or at least 90% or more of a set of selected differentially expressed genes or for the entire set of selected differentially expressed genes.

As described herein, a differential translational or expression profile between a first sample and a control may be “comparable” to a differential translational or expression profile between a second sample and the control (e.g., the differential profile between a hyperproliferative disease sample and the hyperproliferative disease sample treated with a known active compound may be comparable to the differential profile between the hyperproliferative disease sample and the hyperproliferative disease sample contacted with a MNK inhibitor). In certain embodiments, a test differential translational or expression profile is “comparable to” a reference differential translational profile when at least 99%, 95%, 90%, 80%, 70%, 60%, 50%, 25%, or 10% of a selected portion of differentially translated or expressed genes, a majority of differentially translated or expressed genes, or all differentially translated or expressed genes show a translational profile within 75%, 70%, 65%, 60%, 55%, 50%, 45%, 40%, 35%, 30%, or 25%, respectively, of their corresponding genes in the reference translational or expression profile. In further embodiments, a differential translational or expression profile comprising a selected portion of the differentially translated or expressed genes or all the differentially translated or expressed genes has a differential translational or expression profile “comparable to” the differential translational or expression profile of the same genes in a reference differential translational or expression profile when the amount of protein translated in the experimental and reference differential translational or expression profiles are within about 3.0 log₂, 2.5 log₂, 2.0 log₂, 1.5 log₂, 1.0 log₂, 0.5 log₂, 0.2 log₂ or closer. In still further embodiments, a differential translational or expression profile comprising a selected portion of the differentially translated or expressed genes or all the differentially translated or expressed genes has a differential translational or expression profile “comparable to” the differential translational or expression profile of the same genes in a reference differential translational or expression profile when the amount of protein translated in the experimental and reference differential translational or expression profiles differs by no more than about 50%, 45%, 40%, 35%, 30%, 25%, 20%, 15%, 10%, 5%, 1% or less.

“Treatment,” “treating” or “ameliorating” refers to medical management of a disease, disorder, or condition of a subject (i.e., patient), which may be therapeutic, prophylactic/preventative, or a combination treatment thereof. A treatment may improve or decrease the severity at least one symptom of hyperproliferative disease, delay worsening or progression of a disease, delay or prevent onset of additional associated diseases. “Reducing the risk of developing a hyperproliferative disease” refers to preventing or delaying onset of a hyperproliferative disease or reoccurrence of one or more symptoms of the hyperproliferative disease.

A “therapeutically effective amount (or dose)” or “effective amount (or dose)” of a compound refers to that amount sufficient to result in amelioration of one or more symptoms of the disease being treated in a statistically significant manner. When referring to an individual active ingredient administered alone, a therapeutically effective dose refers to that ingredient alone. When referring to a combination, a therapeutically effective dose refers to combined amounts of the active ingredients that result in the therapeutic effect, whether administered serially or simultaneously.

The term “pharmaceutically acceptable” refers to molecular entities and compositions that do not produce allergic or other serious adverse reactions when administered to a subject using routes well-known in the art.

A “subject in need” refers to a subject at risk of, or suffering from, a disease, disorder or condition (e.g., hyperproliferative disease) that is amenable to treatment or amelioration with a compound or a composition thereof provided herein. Subjects in need of administration of therapeutic agents as described herein include subjects suspected of having a cancer, subjects presenting with an existing cancer, or subjects receiving a cancer vaccine. A subject may be any organism capable of developing cancer or being infected, such as humans, pets, livestock, show animals, zoo specimens, or other animals. For example, a subject may be a human, a non-human primate, dog, cat, rabbit, horse, or the like. In certain embodiments, a subject in need is a human.

The “percent identity” between two or more nucleic acid sequences is a function of the number of identical positions shared by the sequences (i.e., % identity=number of identical positions/total number of positions x 100), taking into account the number of gaps, and the length of each gap that needs to be introduced to optimize alignment of two or more sequences. The comparison of sequences and determination of percent identity between two or more sequences can be accomplished using a mathematical algorithm, such as BLAST and Gapped BLAST programs at their default parameters (e.g., Altschul et al., J. Mol. Biol. 215:403, 1990; see also BLASTN at www.ncbi.nlm.nih.gov/BLAST).

A “conservative substitution” is recognized in the art as a substitution of one amino acid for another amino acid that has similar properties. Exemplary conservative substitutions are well known in the art (see, e.g., WO 97/09433, p. 10; Lehninger, Biochemistry, 2^{nd} Edition; Worth Publishers, Inc. NY:NY (1975), pp.71-'7′7; Lewin, Genes IV, Oxford University Press, NY and Cell Press, Cambridge, Mass. (1990), p. 8).

### MNK Inhibitors

Exemplary MNK inhibitors can inhibit both MNK1 and MNK2 kinase activity. In certain embodiments, a MNK inhibitor selectively inhibits MNK1 kinase activity over MNK2 kinase activity, or selectively inhibits MNK2 kinase activity over MNK1 kinase activity. In other embodiments, a MNK inhibitor selectively inhibits kinase activity of full length isoforms MNK1a and MNK2a over the kinase activity of MNK1b and MNK2b. In further embodiments, a MNK inhibitor selectively inhibits either MNK1 kinase activity or MNK2 kinase activity. In still further embodiments, a MNK inhibitor selectively inhibits kinase activity of any one of full length isoforms MNK1a, MNK1b, MNK2a, or MNK2b.

In further embodiments, a MNK inhibitor may be a compound, antisense molecule, ribozyme, RNAi molecule, or low molecular weight organic molecule. In certain embodiments, an MNK inhibitor is a compound having the following structure (I):

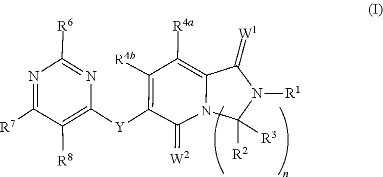

or a stereoisomer, tautomer or pharmaceutically acceptable salt thereof wherein:

W¹ and W² are independently O, S or N—OR′, where R′ is lower alkyl;

Y is —N(R⁵)—, —O—, —S—, —C(O)—, —S═O, —S(O)₂—, or —CHR⁹—;

R¹ is hydrogen, lower alkyl, cycloalkyl or heterocyclyl wherein any lower alkyl, cycloalkyl or heterocyclyl is optionally substituted with 1, 2 or 3 J groups;

n is 1, 2 or 3;

R² and R³ are each independently hydrogen, alkyl, alkenyl, alkynyl, aryl, araalkylene, heteroaryl, heteroarylalkylene, cycloalkyl, cycloalkylalkylene, heterocyclyl, or heterocyclylalkylene, wherein any alkyl, aryl, araalkylene, heteroaryl, heteroarylalkylene, cycloalkyl, cycloalkylalkylene, heterocyclyl, or heterocyclylalkylene, is optionally substituted with 1, 2 or 3 J groups;

or R² and R³ taken together with the carbon atom to which they are attached form a cycloalkyl or heterocyclyl, wherein any cycloalkyl or heterocyclyl is optionally substituted with 1, 2 or 3 J groups;

R^{4a} and R^{4b} are each independently hydrogen, halogen, hydroxyl, thiol, hydroxyalkylene, cyano, alkyl, alkoxy, acyl, thioalkyl, alkenyl, alkynyl, cycloalkyl, aryl, or heterocyclyl;

R⁵ is hydrogen, cyano, or lower alkyl;

or R⁵ and R⁸ taken together with the atoms to which they are attached form a fused heterocyclyl optionally substituted with 1, 2 or 3 J groups;

R⁶, R⁷ and R⁸ are each independently hydrogen, hydroxy, halogen, cyano, amino, alkyl, alkenyl, alkynyl, alkoxy, cycloalkyl, cycloalkylalkylene, cycloalkylalkenylene, alkylaminyl, alkylcarbonylaminyl, cycloalkylcarbonylaminyl, cycloalkylaminyl, heterocyclylaminyl, heteroaryl, or heterocyclyl, and wherein any amino, alkyl, alkenyl, alkynyl, alkoxy, cycloalkyl, cycloalkylalkylene, cycloalkylalkenylene, amino, alkylaminyl, alkylcarbonylaminyl, cycloalkylcarbonylaminyl, cycloalkylaminyl, heterocyclylaminyl, heteroaryl, or heterocyclyl is optionally substituted with 1, 2 or 3 J groups;

or R⁷ and R⁸ taken together with the atoms to which they are attached form a fused heterocyclyl or heteroaryl optionally substituted with 1, 2 or 3 J groups;

J is —SH, —SR⁹, —S(O)R⁹, —S(O)₂R⁹, —S(O)NH₂, —S(O)NR⁹R⁹, —NH₂, —NR⁹R⁹, —COOH, —C(O)OR⁹, —C(O)R⁹, —C(O)-NH₂, —C(O)—NR⁹R⁹, hydroxy, cyano, halogen, acetyl, alkyl, lower alkyl, alkenyl, alkynyl, alkoxy, haloalkyl, thioalkyl, cyanoalkylene, alkylaminyl, NH₂—C(O)-alkylene , NR⁹R⁹—C(O)-alkylene, —CHR⁹—C(O)-lower alkyl, —C(O)-lower alkyl, alkylcarbonylaminyl, cycloalkyl, cycloalkylalkylene, cycloalkylalkenylene, cycloalkylcarbonylaminyl, cycloalkylaminyl, —CHR⁹—C(O)-cycloalkyl, —C(O)-cycloalkyl, —CHR⁹—C(O)-aryl, —CHR⁹-aryl, —C(O)-aryl, —CHR⁹—C(O)-heterocycloalkyl, —C(O)-heterocycloalkyl, heterocyclylaminyl, or heterocyclyl; or any two J groups bound to the same carbon or hetero atom may be taken together to form oxo; and

R⁹ is hydrogen, lower alkyl or —OH.

In one embodiment of structure (I), the present disclosure provides a compound having the following structure (Ia), as well as stereoisomers, tautomers or pharmaceutically acceptable salts thereof.

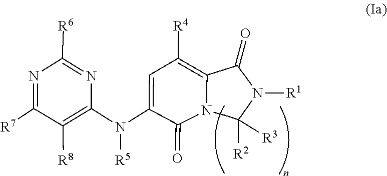

For Formula Ia compounds, substituent le is hydrogen or lower alkyl and subscript n is 1, 2 or 3. Substituents R² and R³ in Formula Ia are each independently hydrogen, alkyl, cycloalkyl, cycloalkylalkylene, heterocyclyl or heterocyclylalkyl, and any such alkyl, cycloalkyl, cycloalkylalkylene, heterocyclyl or heterocyclylalkyl can optionally be substituted with 1, 2 or 3 J groups.

Substitutents R² and R³ in Formula Ia when taken together with the carbon atom to which they are attached can form a cycloalkyl or heterocyclyl, wherein any such cycloalkyl or heterocyclyl is optionally substituted with 1, 2 or 3 J groups. In Formula Ia, R⁴a is hydrogen, halogen, hydroxy, alkyl, alkoxy, thioalkyl, alkenyl or cycloalkyl and substituent R⁵ is hydrogen or lower alkyl.

Alternatively, substituent groups R⁵ and R⁸ taken together with the atoms to which they are attached form a fused heterocyclyl that is optionally substituted with 1, 2 or 3 J groups.

In one embodiment, substituents R⁶, R⁷ and R⁸ are independently and at each occurrence hydrogen, halogen, alkyl, alkenyl, cycloalkly, cycloalkylalkyl, cycloalkylalkenyl, amino, alkylaminyl, alklycarbonylaminyl, cycloalkylcarbonylaminyl, alkylaminyl or cycloalkylaminyl, and any such alkyl, alkenyl, cycloalkly, cycloalkylalkyl, cycloalkylalkenyl, amino, alkylaminyl, alklycarbonylaminyl, cycloalkylcarbonylaminyl, alkylaminyl or cycloalkylaminyl is optionally substituted with 1, 2 or 3 J groups. For some compounds in accordance with Formula Ia, R⁷ and R⁸ taken together with the atoms to which they are attached form a fused heterocyclyl unsubstituted or substituted with 1, 2 or 3 J groups.

Variable J in Formula Ia is —SH, —SR⁹, —S(O) R⁹, —S(O)₂R⁹, —S(O)NH₂, —S(O)NR⁹R⁹, —NH₂, —NR⁹R⁹, —COOH, —C(O)OR⁹, —C(O)R⁹, —C(O)—NH₂, —C(O)—NR⁹R⁹, hydroxy, cyano, halogen, acetyl, alkyl, lower alkyl, alkenyl, alkynyl, alkoxy, haloalkyl, thioalkyl, cyanoalkylene, alkylaminyl, NH₂—C(O)-alkylene , NR⁹R⁹—C(O)-alkylene, —CHR⁹—C(O)-lower alkyl, —C(O)-lower alkyl, alkylcarbonylaminyl, cycloalkyl, cycloalkylalkylene, cycloalkylalkenylene, cycloalkylcarbonylaminyl, cycloalkylaminyl, —CHR⁹—C(O)-cycloalkyl, —C(O)-cycloalkyl, —CHR⁹—C(O)-aryl, —CHR⁹-aryl, —C(O)-aryl, —CHR⁹—C(O)-heterocycloalkyl, —C(O)-heterocycloalkyl, heterocyclylaminyl, or heterocyclyl. For some of the inventive compounds according to Formula Ia, any two J groups bound to the same carbon or hetero atom may be taken together to form an oxo group.

In some embodiments, variable J in Formula Ia is halogen, amino, alkyl, haloalkyl, alkylaminyl, cycloalkyl or heterocyclyl. Alternatively, for certain Formula Ia compounds, any two J groups when bound to the same carbon or hetero atom may be taken together to form oxo group.

Further MNK inhibitors are compounds according to Formula IIa, illustrated below, where variable Y is —N(R⁵)— and subscript “n” is 1.

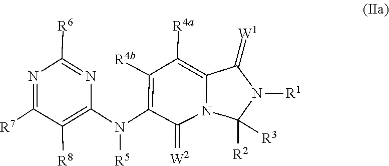

According to one embodiment, variable Y in Formula I is —O—, —S—, —C(O)—, sulfoxide, sulfone, —CHR⁹— or —CH₂—, subscript “n” is 1 and the inventive compounds conform to Formula IIb. When “Y” is —CHR⁹— in Formula IIb, substituent R⁹ is hydrogen, lower alkyl or hydroxy.

Substitute Specification (Clean Copy)

Attorney Docket No.: 050040-515C01US

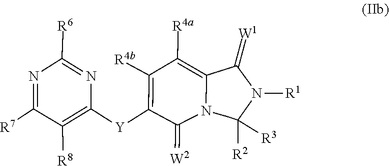

In more MNK inhibitor embodiments, variable “Y” in Formula I is —N(R⁵)—, subscript “n” is 2 or 3 and the compounds conform to Formula Ma or Formula IVa, respectively:

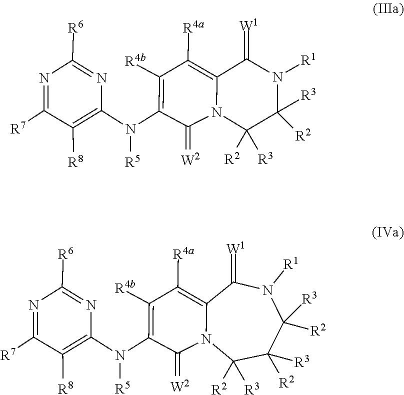

Alternatively, in certain embodiments, variable “Y” in Formula I is —O—, —S—, —C(O)—, sulfoxide, sulfone, —CHR⁹— or —CH₂—, “n” is 2 or 3 and the compounds conform to Formula Mb and Formula IVb, respectively: When “Y” is —CHR⁹— in Formula Mb or Formula IVb, substituent R⁹ is either hydrogen, lower alkyl or hydroxy.

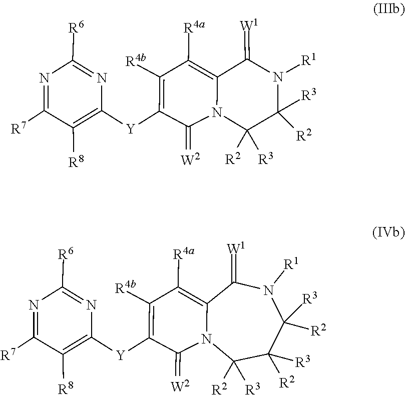

For MNK inhibitor compounds according to Formulae II, IIb, IIIa, IIIb, IVa and IVb, variables W¹ and W² are both oxo. In certain embodiments for compounds according to Formulae IIa, IIb, IIIa, IIIb, IVa and IVb, W¹ is oxo and W² is thione group. According to one embodiment, Formulae IIa, IIb, IIIa, IIIb, IVa and IVb compounds comprise an oxo at W¹ and a ═N—OR′ group at W². Also encompassed within the scope of the present MNK inhibitors are Formulae IIa, IIb, IIIa, IIIb, IVa and IVb compounds having a thione group at W¹ and an oxo group at W².

For Formulae IIa, IIb, IIIa, IIIb, IVa and IVb compounds, each of substituents R² and R³ can be the same in which case the carbon atom which R² and R³ are attached is not a chiral carbon. In certain embodiments, however, substituents R² and R³ are different. Thus, the carbon atom to which R² and R³ are attached is chiral and the resulting compound will have stereoisomers.

In certain MNK inhibitor embodiments, each R² and R³ in Formulae IIa, IIb, IIIa, IIIb, IVa and IVb is hydrogen. Alternatively, one of R² or R³ groups in Formulae IIa, IIb, IIIa, IIIb, IVa and IVb is hydrogen and the other group is alkyl optionally substituted with 1, 2 or 3 J groups. For certain compounds according to Formulae IIa, IIb, IIIa, IIIb, IVa and IVb, R² and R³ are both alkyl groups that are optionally substituted with 1, 2 or 3 J groups.

For some compounds in accordance with Formula IIa or Formula IIb, R² is alkyl and R³ is alkyl substituted with 1, 2 or 3 J groups. Exemplary of this category of Formula

Ha and Formula lib compounds are the following: compounds with substituent R² as alkyl and R³ is haloalkyl; compounds with substituent compounds with substituent R² as alkyl and R³ is cycloalkyl optionally substituted with 1, 2 or 3 J groups; compounds with substituent R² as alkyl and R³ is cyclopentyl optionally substituted with 1, 2 or 3 J groups; compounds with substituent R² as alkyl and R³ is aryl optionally substituted with 1, 2 or 3 J groups; compounds with substituent R² as alkyl and R³ is phenyl optionally substituted with 1, 2 or 3 J groups; compounds with substituent R² as alkyl and R³ is cycloalkylalkylene optionally substituted with 1, 2 or 3 J groups; compounds with substituent R² as alkyl and R³ is aralkylene optionally substituted with 1, 2 or 3 J groups; compounds with substituent R² as alkyl and R³ is benzyl optionally substituted with 1, 2 or 3 J groups; compounds with substituent R² as alkyl and R³ is heterocyclyl optionally substituted with 1, 2 or 3 J groups; compounds with substituent R² as alkyl and R³ is heteroaryl optionally substituted with 1, 2 or 3 J groups; compounds with substituent R² as alkyl and R³ is thiophenyl, thiazolyl or pyridinyl; compounds with substituent R² as alkyl and R³ is heterocyclylalkylene substituted or substituted with 1, 2 or 3 J groups; or compounds with substituent R² as alkyl and R³ is heteroarylalkylene optionally substituted with 1, 2 or 3 J groups.

In some embodiments, for compounds according to Formulae IIa, IIb, IIIa, IIIb, IVa and IVb, each R² and R³ are independently hydrogen, alkyl, cycloalkyl, cycloalkylalkylene, heterocyclyl or heterocyclylalkylene, and any such alkyl, cycloalkyl, cycloalkylalkylene, heterocyclyl or heterocyclylalkylene can optionally be substituted with 1, 2 or 3 J groups, idependently selected from the group consisting of halogen, amino, alkylaminyl and alkyl.

For certain Formulae IIIa, IIIb, IVa and IVb compounds, R² and R³ together with the carbon atom to which they are attached form a cycloalkyl or heterocyclyl ring.

Also contemplated are Formula I compounds where Y is —N(R⁵)-, subscript “n” is 1 and R² and R³ together with the carbon atom to which they are attached form a cycloalkyl or heterocyclyl ring “A.” Such compounds conform to Formula Va and the cycloalkyl or heterocyclyl ring “A” may optionally be substituted with 1, 2 or 3 J groups.

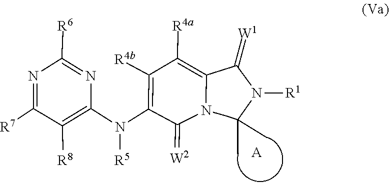

Alternatively, in some embodiments Y in Formula I is —O—, —S—, —C(O)—, sulfoxide, sulfone, —CHR⁹- or —CH₂—, “n” is 1 and R² and R³ together with the carbon atom to which they are attached form a cycloalkyl or heterocyclyl ring A. Such compounds conform to Formula Vb and the cycloalkyl or heterocyclyl ring “A” may optionally be substituted with 1, 2 or 3 J groups. When “Y” is —CHR⁹— in Formula Vb, substituent R⁹ is either hydrogen, lower alkyl or hydroxy.

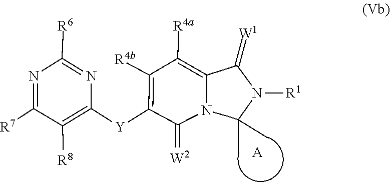

For Formula Va and Formula Vb compounds, W¹ and W² are both oxo and ring A is a cycloalkyl optionally substituted with 1, 2 or 3 J groups. Also contemplated are Formula Va and Formula Vb compounds for which ring A is a fused cycloalkyl optionally substituted with 1, 2 or 3 J groups; ring A is a cycloalkyl optionally substituted with 1, 2 or 3 J groups; ring A is a cyclobutyl, cyclopentyl or cyclohexyl optionally substituted with 1, 2 or 3 J groups, for example, J groups selected from the group consisting of halogen, amino, alkylaminyl and alkyl.

For some embodiments, ring A of a Formula Va or a Formula Vb is a heterocyclyl optionally substituted with 1, 2 or 3 J groups. Exemplary of such heterocyclyl groups are pyrrolidinyl, piperidinyl, tetrahydropyranyl, thietanyl or azetidinyl. In one embodiment, each of the above exemplified heterocyclyl may optionally be substituted with 1, 2 or 3 J groups. For certain Formula Va or a Formula Vb compounds ring A is a cycloalkyl substituted with at least 2J groups attached to the same carbon atom of the cycloalkyl, and the two J groups attached to the same carbon taken together form oxo group. In another embodiment, ring A of a Formula Va or a Formula Vb is a heterocyclyl substituted with at least 2J groups that are attached to the same hetero atom and wherein such 2 J groups taken together to form oxo. For some Formula Va or a Formula Vb compounds the cycloalkyl or heterocyclyl ring A is substituted with J groups selected from from the group consisting of halogen, cyano, hydroxy, trifluoromethyl, N-methyl amino, methyl, difluoroethylene, and methylenenitrile.

The present invention also provides compounds in accordance with Formula VI or its stereoisomers, tautomers or pharmaceutically acceptable salts. Formula VI is a sub-genus of Formula I in which Y is —N(R⁵)— and substituent groups R⁵ and R⁸ together with the atoms to which they are attached form a heterocycle ring B which may optionally be substituted with 1, 2 or 3 J groups.

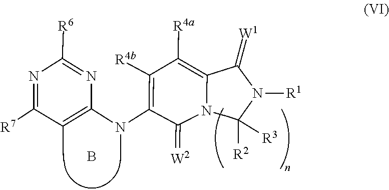

Also encompassed within the scope of the present MNK inhibitors are Formula I compounds in which variable “Y” is —N(R⁵)—, and substituent groups R⁷ and R⁸ together with the atoms to which they are attached form a fused ring C. Such compounds or the stereoisomer, tautomer or pharmaceutically acceptable salt conform to Formula VIIa . For Formula VIIa compounds, ring C may optionally be substituted with 1, 2 or 3 J groups.

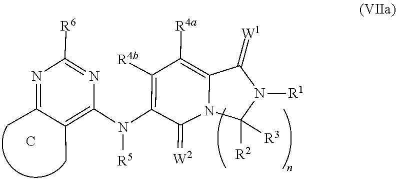

According to one embodiment, variable “Y” in Formula I is —O—, —S—, —C(O)—, sulfoxide, sulfone, —CHR⁹— or —CH₂—, and substituent groups R⁷ and R⁸ together with the atoms to which they are attached form a fused ring C. Such compounds and their stereoisomers, tautomers or pharmaceutically acceptable salts conform to Formula VIIb. For Formula VIIb compounds where “Y” is —CHR⁹—, substituent R⁹ can be hydrogen, lower alkyl or hydroxy.

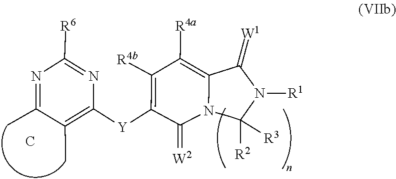

For Formula VIIb compounds, fused ring C may optionally be substituted with 1, 2 or 3 J groups. In one MNK inhibitor embodiment, W¹ and W² are both oxo for Formula VI, Formula VIla and Formula VIIb compounds. MNK inhibitors of this disclosure are further directed to Formulae I, Ia, IIa, IIb, IIIa, IIIb, IVa, IVb, Va, Vb, VI, VIIa and VIIb compounds where le is hydrogen or a lower alkyl group selected from methyl, ethyl, propyl, butyl, iso-propyl, sec-butyl, or tert-butyl, for example, compounds with le as methyl.

For certain Formulae I, Ia, IIa, IIb, IIIa, IIIb, IVa, IVb, Va, Vb, VI, VIIa and VIIb compounds, R⁴a is selected from the group consisting of hydrogen, halogen, alkyl, alkoxy, thioalkyl, alkenyl, and cycloalkyl while substituent R^{ob} is hydrogen or halogen. R⁵ in Formulae I, Ia, IIa, IIb, IIIa, IIIb, IVa, IVb, Va, Vb, VI, VIIa and VIIb is hydrogen or lower alkyl, while substituents R⁶, R⁷ and R⁸ are hydrogen.

In certain embodiments of this disclosure, R⁶ and R⁷ in Formula VI are both hydrogen, while for certain Formula VIIa and Formula VIIb compounds R⁶ is hydrogen.

MNK inhibitors of this disclosure are further directed to Formulae I, Ia, IIa, IIb, IIIa, IIIb, IVa, IVb, Va, and Vb compounds where substituent groups R⁶ and R⁸ are both hydrogen, and R₇ is selected from the group consisting of hydroxy, halogen, cyano, alkyl, alkenyl, alkynyl, alkoxy, cycloalkyl cycloalkylalkylene, cycloalkylalkenylene, amino, alkylaminyl, alkylcarbonylaminyl, cycloalkylcarbonylaminyl, cycloalkylaminyl, heterocyclylaminyl, heteroaryl, and heterocyclyl. For these compounds, any alkyl, alkenyl, alkynyl, alkoxy, cycloalkyl, cycloalkylalkylene, cycloalkylalkenylene, amino, alkylaminyl, alkylcarbonylaminyl, cycloalkylcarbonylaminyl, cycloalkylaminyl, heterocyclylaminyl, heteroaryl, or heterocyclyl is optionally substituted with 1, 2 or 3 J groups. In certain embodiments, R₇ is selected from the group consisting of alkyl, cycloalkyl, cycloalkylalkylene, cycloalkylalkenylene, amino, alkylaminyl, alklycarbonylaminyl, cycloalkylcarbonylaminyl, heterocyclylaminyl, heteroaryl, heterocyclyl and cycloalkylaminyl. For such compounds any alkyl, alkenyl, cycloalkyl, cycloalkylalkylene, cycloalkylalkenylene, amino, alkylaminyl, alklycarbonylaminyl, cycloalkylcarbonylaminyl, heterocyclylaminyl, heteroaryl, heterocyclyl or cycloalkylaminyl may optionally be substituted with 1, 2 or 3 J groups. Thus, certain embodiments provide Formulae I, Ia, IIa, IIb, IIIa, IIIb, IVa, IVb, Va, and Vb compounds where substituent groups R⁶ and R⁸ are both hydrogen, and R₇ is amino; substituent groups R⁶ and R⁸ are both hydrogen, and R₇ is alkylaminyl; substituent groups R⁶ and R⁸ are both hydrogen, and R₇ is —NHCH₃; substituent groups R⁶ and R⁸ are both hydrogen, and R₇ is cycloalkyl, for example cyclopropyl; substituent groups R⁶ and R⁸ are both hydrogen, and R₇ is cycloalkylaminyl substituted with 1 to 3 J groups, for instance halogens.

In one embodiment, for compounds in accordance with Formulae I, Ia, IIa, IIb, IIIa, IIIb, IVa, IVb, Va, and Vb, substituent groups R⁶ and R⁸ are both hydrogen, and R₇ is selected from the group consisting of —NHCH(CF₃)cyclopropyl, cycloalkylcarbonylaminyl, —NHC(O)cyclopropyl, cycloalkylalkenylene, and —CH═CHcyclopropyl.

For any compound in accordance with Formulae I, Ia, IIa, IIb, IIIa, IIIb, IVa, IVb, Va, Vb, VI, VIIa, and VIIb, J is —SH, —SR⁹, —S(O)R⁹, —S(O)₂ R⁹, —S(O)NH₂, —S(O)NR⁹R⁹, —NH₂, -NR⁹R⁹, —COOH, —C(O)OR⁹, —C(O)R⁹, —C(O)-NH₂, —C(O)-NR⁹R⁹, hydroxy, cyano, halogen, acetyl, alkyl, lower alkyl, alkenyl, alkynyl, alkoxy, haloalkyl, thioalkyl, cyanoalkylene, alkylaminyl, NH₂—C(O)-alkylene, NR⁹R⁹—C(O)-alkylene, —CHR⁹—C(O)-lower alkyl, —C(O)-lower alkyl, alkylcarbonylaminyl, cycloalkyl, cycloalkylalkylene, cycloalkylalkenylene, cycloalkylcarbonylaminyl, cycloalkylaminyl, —CHR⁹—C(O)-cycloalkyl, —C(O)-cycloalkyl, —CHR⁹—C(O)-aryl, —CHR⁹-aryl, —C(O)-aryl, —CHR⁹—C(O)-heterocycloalkyl, —C(O)-heterocycloalkyl, heterocyclylaminyl, or heterocyclyl and R⁹ is hydrogen, lower alkyl or -OH. Additionally, when two J groups bound to the same carbon or hetero atom they may be taken together to form oxo.

For certain compounds according to Formulae I, Ia, IIa, IIb, IIIa, IIIb, IVa, IVb, Va, Vb, VI, VIIa, and VIIb, J is halogen, hydroxy, alkyl, alkenyl, alkynyl or cyanoalkylene. Illustrative alkyl or alkylene chains are those having Ci-C₁₀ carbon atoms, C₁-C₈ carbon atoms, C₁-C₆ carbon atoms, C₁-C₄ carbon atoms, C₁-C₃ carbon atoms as well as ethyl and methyl groups. Alternatively, when J is alkenyl, or alkynyl, the carbon chain has at least one double or triple bond respectively and C₂-C₁₀ carbon atoms, C₂-C₈ carbon atoms, C₂-C₆ carbon atoms, C₂-C₄ carbon atoms, or C₂-C₃ carbon atoms.

A MNK inhibitor of Formula (I), as well as Formulae Ia, IIa, IIb, IIIa, IIIb, IVa, IVb, Va, Vb VI, VIIa and VIIb, may be isotopically-labelled by having one or more atoms replaced by an atom having a different atomic mass or mass number. Examples of isotopes that can be incorporated into the compounds of structure (I) include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabelled compounds may be useful to help determine or measure the effectiveness of the compounds, by characterizing, for example, the site or mode of action, or binding affinity to pharmacologically important site of action. Certain isotopically-labelled compounds of Formula (I), for example, those incorporating a radioactive isotope, are useful in drug or substrate tissue distribution studies. The radioactive isotopes tritium, i.e., ³H, and carbon-14, i.e ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula (I), as well as Formulae Ia, IIa, IIb, IIIa, IIIb, IVa, IVb, Va, Vb VI, VIIa and VIIb, can generally be prepared by conventional techniques known to those skilled in the art or by processes analogous to those described in the Preparations and Examples as set out in U.S. patent application Ser. No. 14/748,990 filed Jun. 24, 2015 and entitled “MNK Inhibitors and Methods Related Thereto,” which compounds and synthetic methods are incorporated herein in their entirety, using an appropriate isotopically-labeled reagent in place of the non-labeled reagent previously employed.

Embodiments of this disclosure are also meant to encompass the in vivo metabolic products of the MNK inhibitors of Formulae I, Ia, IIa, IIb, IIIa, IIIb, IVa, IVb, Va, Vb VI, VIIa and VIIb. Such products may result from, for example, the oxidation, reduction, hydrolysis, amidation, esterification, and the like of the administered compound, primarily due to enzymatic processes. Accordingly, the instant disclosure includes compounds produced by a process comprising administering a MNK inhibitor of this disclosure to a mammal for a period of time sufficient to yield a metabolic product thereof. Such products are typically identified by administering a radiolabelled MNK inhibitor as described herein in a detectable dose to an animal, such as rat, mouse, guinea pig, monkey, or human, allowing sufficient time for metabolism to occur, and isolating conversion products from the urine, blood or other biological samples.

In some embodiments, a MNK inhibitor of any one of compounds according to Formulae I, Ia, IIa, IIb, IIIa, IIIb, IVa, IVb, Va, Vb VI, VIIa and VIIb are in the form of a pharmaceutically acceptable salt, which includes both acid and base addition salts.

To this end, a “pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, or the like.

Similarly, a “pharmaceutically acceptable base addition salt” refers to those salts which retain the biological effectiveness and properties of the free acids, which are not biologically or otherwise undesirable. These salts are prepared by addition of an inorganic base or an organic base to the free acid. Salts derived from inorganic bases include the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, di cyclohexylamine, choline and caffeine.

Often crystallizations produce a solvate of a MNK inhibitor compound of this disclosure. As used herein, the term “solvate” refers to an aggregate that comprises one or more molecules of a compound of the invention with one or more molecules of solvent. A solvent may be water, in which case the solvate may be a hydrate. Alternatively, a solvent may be an organic solvent. Thus, the MNK inhibitor compounds of the present disclosure may exist as a hydrate, including a monohydrate, dihydrate, hemihydrate, sesquihydrate, trihydrate, tetrahydrate or the like, as well as the corresponding solvated forms. The MNK inhibitor compounds of this disclosure may be true solvates, while in other cases, the compounds may merely retain adventitious water or be a mixture of water plus some adventitious solvent.

A “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are non-superimposeable mirror images of one another.

MNK inhibitors of this disclosure, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

The term “tautomer” refers to a proton shift from one atom of a molecule to another atom of the same molecule. For example, when W′ is oxo and le is H, the present disclosure provides tautomers of a Formula I compound as illustrated below:

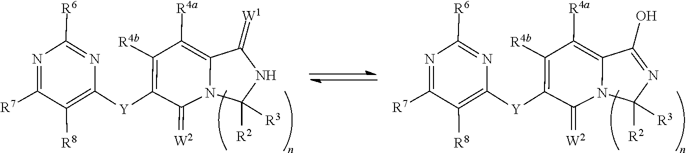

Similar tautomers exists for Formulae I, Ia, IIa, IIb, IIIa, IIIb, IVa, IVb, Va, Vb VI, VIIa and VIIb compounds. The compounds are synthesized using conventional synthetic methods, and more specifically using the general methods and specific synthetic protocols of the Examples found in U.S. patent application Ser. No. 14/748,990, filed Jun. 24, 2015 and entitled “MNK Inhibitors and Methods Related Thereto,” which compounds and synthetic methods are incorporated herein in their entirety.

Representative MNK inhibitor compounds of this disclosure are set forth in Table 1 and in U.S. patent application Ser. No. 14/748,990, filed Jun. 24, 2015 and entitled “MNK Inhibitors and Methods Related Thereto,” which compounds are incorporated herein by reference in their entirety. Similarly, incorporated herein by reference in their entirety are compounds and methods of making the same from U.S. Provisional Patent Application No. 62/247,953 (entitled “Isoindoline, Azaisoindoline, Dihydroindenone and Dihydroazaindenone Inhibitors of MNK1 and MNK2”) and 62/247,966 (entitled “Pyrrolo-, Pyrazolo-, Imidazo-Pyrimidine and Pyridine Compounds that Inhibit MNK1 and MNK2”).

Such compounds are provided for purpose of illustration and not limitation.

TABLE 1
Exemplary MNK Inhibitors

| Cmpd. | |
|---|---|
| No. | Structure |
| 1 | 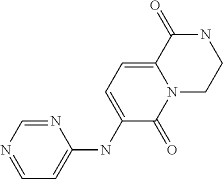 [SMILES: O=C1NCCn2c1ccc(Nc1ccncn1)c2=O] |
| 2 | 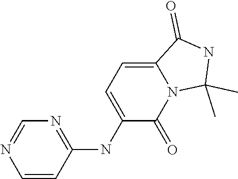 [SMILES: CC1(C)NC(=O)c2ccc(Nc3ccncn3)c(=O)n21] |
| 3 | 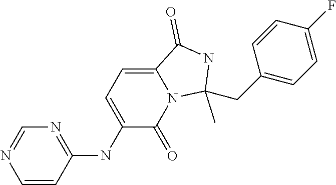 [SMILES: CC1(Cc2ccc(F)cc2)NC(=O)c2ccc(Nc3ccncn3)c(=O)n21] |
| 4 | 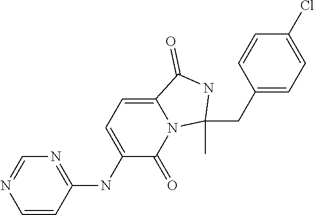 [SMILES: CC1(Cc2ccc(Cl)cc2)NC(=O)c2ccc(Nc3ccncn3)c(=O)n21] |
| 5 | 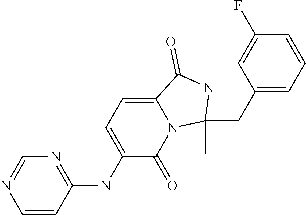 [SMILES: CC1(Cc2cccc(F)c2)NC(=O)c2ccc(Nc3ccncn3)c(=O)n21] |
| 6 | 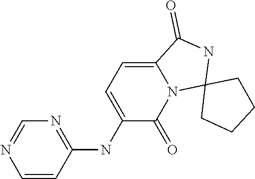 [SMILES: O=C1NC2(CCCC2)n2c1ccc(Nc1ccncn1)c2=O] |
| 7 | 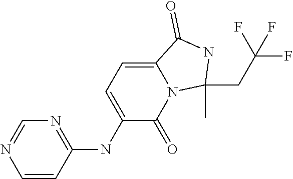 [SMILES: CC1(CC(F)(F)F)NC(=O)c2ccc(Nc3ccncn3)c(=O)n21] |
| 8 | 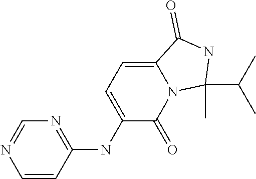 [SMILES: CC(C)C1(C)NC(=O)c2ccc(Nc3ccncn3)c(=O)n21] |
| 9 | 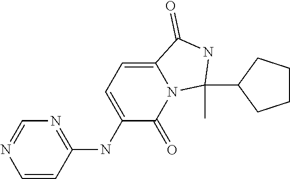 [SMILES: CC1(C2CCCC2)NC(=O)c2ccc(Nc3ccncn3)c(=O)n21] |
| 10 | 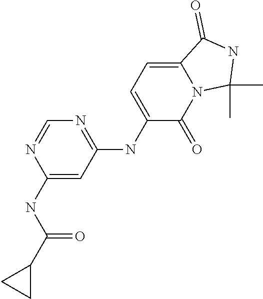 [SMILES: CC1(C)NC(=O)c2ccc(Nc3cc(NC(=O)C4CC4)ncn3)c(=O)n21] |
| 11 | 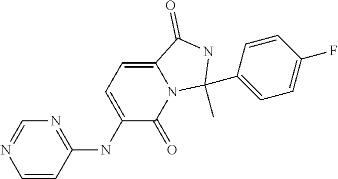 [SMILES: CC1(c2ccc(F)cc2)NC(=O)c2ccc(Nc3ccncn3)c(=O)n21] |
| 12 | 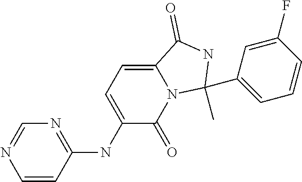 [SMILES: CC1(c2cccc(F)c2)NC(=O)c2ccc(Nc3ccncn3)c(=O)n21] |
| 13 | 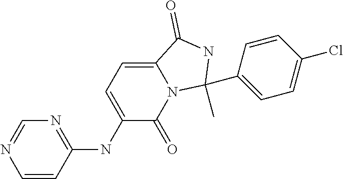 [SMILES: CC1(c2ccc(Cl)cc2)NC(=O)c2ccc(Nc3ccncn3)c(=O)n21] |
| 14 | 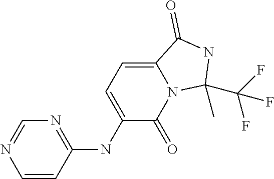 [SMILES: CC1(C(F)(F)F)NC(=O)c2ccc(Nc3ccncn3)c(=O)n21] |
| 15 | 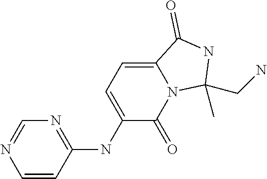 [SMILES: CC1(CN)NC(=O)c2ccc(Nc3ccncn3)c(=O)n21] |
| 16 | 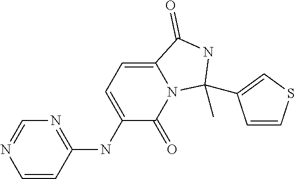 [SMILES: CC1(c2ccsc2)NC(=O)c2ccc(Nc3ccncn3)c(=O)n21] |
| 17 | 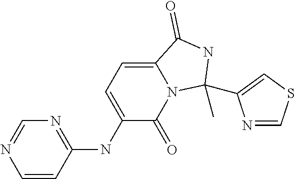 [SMILES: CC1(c2cscn2)NC(=O)c2ccc(Nc3ccncn3)c(=O)n21] |
| 18 | 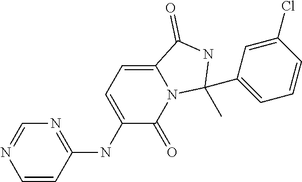 [SMILES: CC1(c2cccc(Cl)c2)NC(=O)c2ccc(Nc3ccncn3)c(=O)n21] |
| 19 | 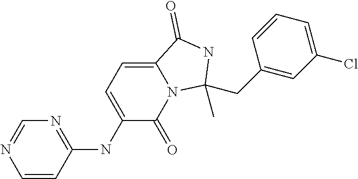 [SMILES: CC1(Cc2cccc(Cl)c2)NC(=O)c2ccc(Nc3ccncn3)c(=O)n21] |
| 20 | 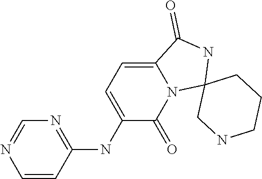 [SMILES: O=C1NC2(CCCNC2)n2c1ccc(Nc1ccncn1)c2=O] |
| 21 | 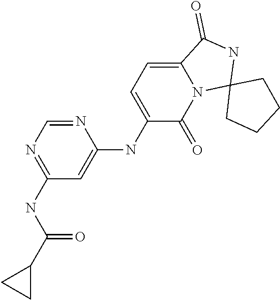 [SMILES: O=C1NC2(CCCC2)n2c1ccc(Nc1cc(NC(=O)C3CC3)ncn1)c2=O] |
| 22 | 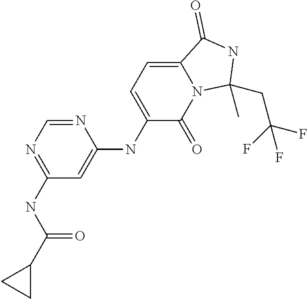 [SMILES: CC1(CC(F)(F)F)NC(=O)c2ccc(Nc3cc(NC(=O)C4CC4)ncn3)c(=O)n21] |
| 23 | 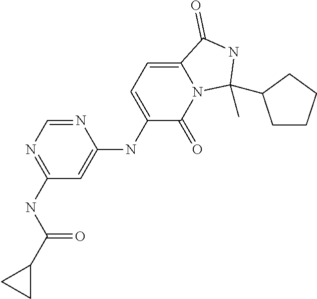 [SMILES: CC1(C2CCCC2)NC(=O)c2ccc(Nc3cc(NC(=O)C4CC4)ncn3)c(=O)n21] |
| 24 | 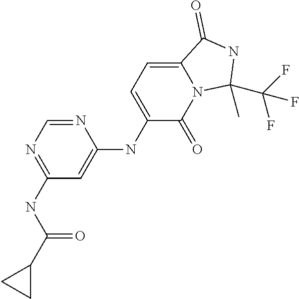 [SMILES: CC1(C(F)(F)F)NC(=O)c2ccc(Nc3cc(NC(=O)C4CC4)ncn3)c(=O)n21] |
| 25 | 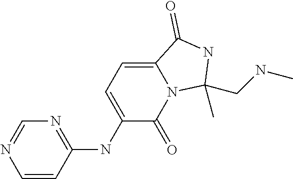 [SMILES: CNCC1(C)NC(=O)c2ccc(Nc3ccncn3)c(=O)n21] |
| 26 | 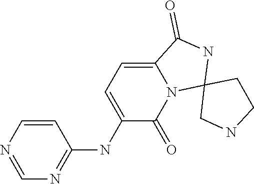 [SMILES: O=C1NC2(CCNC2)n2c1ccc(Nc1ccncn1)c2=O] |
| 27 | 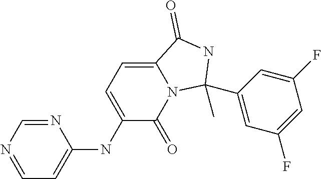 [SMILES: CC1(c2cc(F)cc(F)c2)NC(=O)c2ccc(Nc3ccncn3)c(=O)n21] |
| 28 | 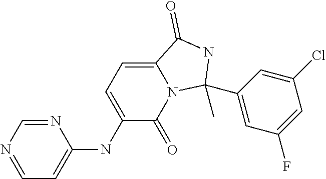 [SMILES: CC1(c2cc(F)cc(Cl)c2)NC(=O)c2ccc(Nc3ccncn3)c(=O)n21] |
| 29 | 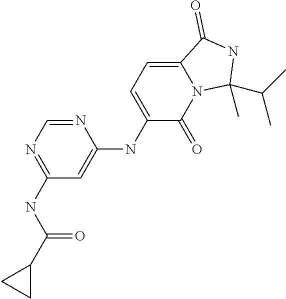 [SMILES: CC(C)C1(C)NC(=O)c2ccc(Nc3cc(NC(=O)C4CC4)ncn3)c(=O)n21] |
| 30 | 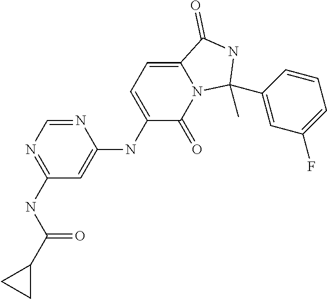 [SMILES: CC1(c2cccc(F)c2)NC(=O)c2ccc(Nc3cc(NC(=O)C4CC4)ncn3)c(=O)n21] |
| 31 | 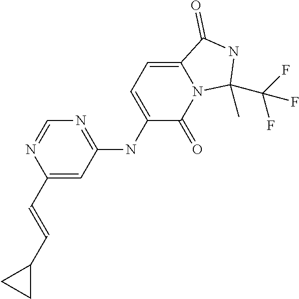 [SMILES: CC1(C(F)(F)F)NC(=O)c2ccc(Nc3cc(/C=C/C4CC4)ncn3)c(=O)n21] |
| 32 | 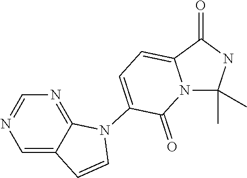 [SMILES: CC1(C)NC(=O)c2ccc(-n3ccc4cncnc43)c(=O)n21] |
| 33 | 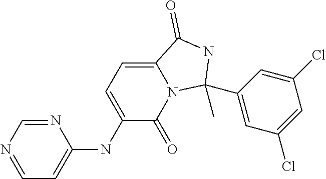 [SMILES: CC1(c2cc(Cl)cc(Cl)c2)NC(=O)c2ccc(Nc3ccncn3)c(=O)n21] |
| 34 | 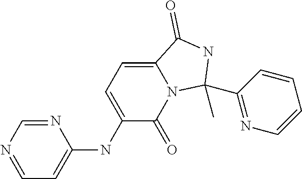 [SMILES: CC1(c2ccccn2)NC(=O)c2ccc(Nc3ccncn3)c(=O)n21] |
| 35 | 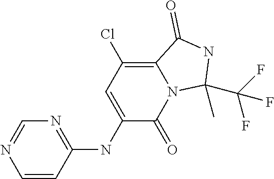 [SMILES: CC1(C(F)(F)F)NC(=O)c2c(Cl)cc(Nc3ccncn3)c(=O)n21] |
| 36 | 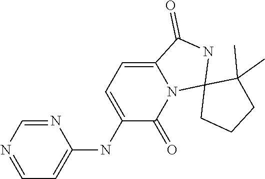 [SMILES: CC1(C)CCCC12NC(=O)c1ccc(Nc3ccncn3)c(=O)n12] |
| 37 | 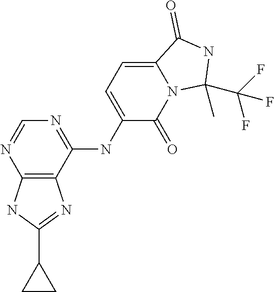 [SMILES: CC1(C(F)(F)F)NC(=O)c2ccc(Nc3ncnc4[nH]c(C5CC5)nc34)c(=O)n21] |
| 38 | 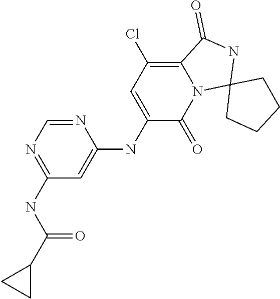 [SMILES: O=C1NC2(CCCC2)n2c1c(Cl)cc(Nc1cc(NC(=O)C3CC3)ncn1)c2=O] |
| 39 | 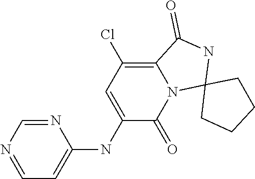 [SMILES: O=C1NC2(CCCC2)n2c1c(Cl)cc(Nc1ccncn1)c2=O] |
| 40 | 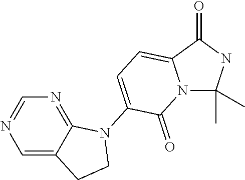 [SMILES: CC1(C)NC(=O)c2ccc(N3CCc4cncnc43)c(=O)n21] |
| 41 | 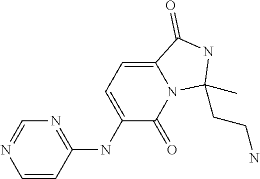 [SMILES: CC1(CCN)NC(=O)c2ccc(Nc3ccncn3)c(=O)n21] |
| 42 | 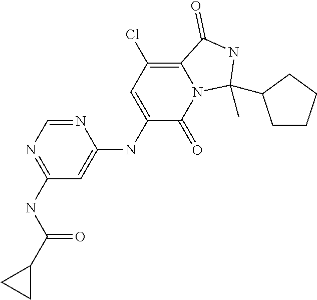 [SMILES: CC1(C2CCCC2)NC(=O)c2c(Cl)cc(Nc3cc(NC(=O)C4CC4)ncn3)c(=O)n21] |
| 43 | 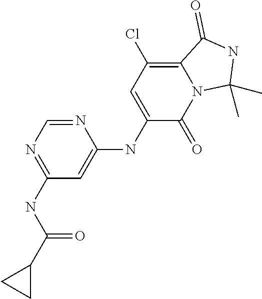 [SMILES: CC1(C)NC(=O)c2c(Cl)cc(Nc3cc(NC(=O)C4CC4)ncn3)c(=O)n21] |
| 44 | 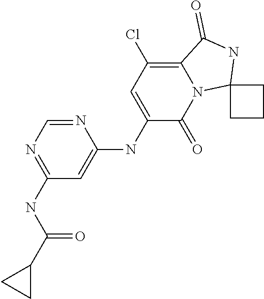 [SMILES: O=C1NC2(CCC2)n2c1c(Cl)cc(Nc1cc(NC(=O)C3CC3)ncn1)c2=O] |
| 45 | 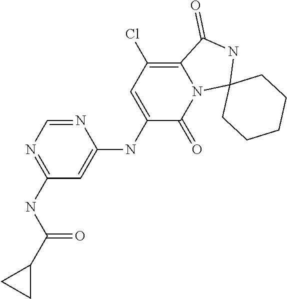 [SMILES: O=C1NC2(CCCCC2)n2c1c(Cl)cc(Nc1cc(NC(=O)C3CC3)ncn1)c2=O] |
| 46 | 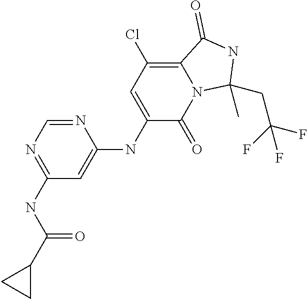 [SMILES: CC1(CC(F)(F)F)NC(=O)c2c(Cl)cc(Nc3cc(NC(=O)C4CC4)ncn3)c(=O)n21] |
| 47 | 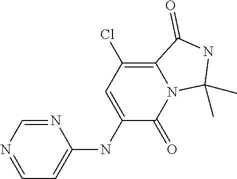 [SMILES: CC1(C)NC(=O)c2c(Cl)cc(Nc3ccncn3)c(=O)n21] |
| 48 | 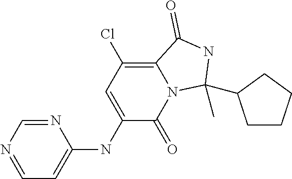 [SMILES: CC1(C2CCCC2)NC(=O)c2c(Cl)cc(Nc3ccncn3)c(=O)n21] |
| 49 | 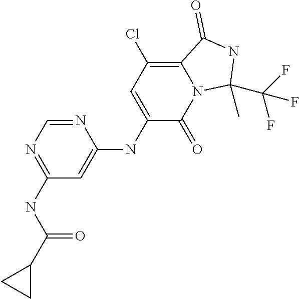 [SMILES: CC1(C(F)(F)F)NC(=O)c2c(Cl)cc(Nc3cc(NC(=O)C4CC4)ncn3)c(=O)n21] |
| 50 | 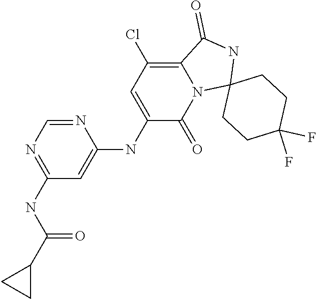 [SMILES: O=C1NC2(CCC(F)(F)CC2)n2c1c(Cl)cc(Nc1cc(NC(=O)C3CC3)ncn1)c2=O] |
| 51 | 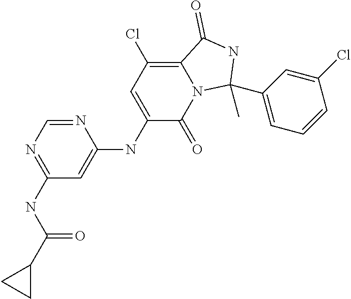 [SMILES: CC1(c2cccc(Cl)c2)NC(=O)c2c(Cl)cc(Nc3cc(NC(=O)C4CC4)ncn3)c(=O)n21] |
| 52 | 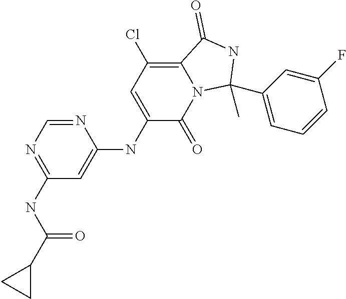 [SMILES: CC1(c2cccc(F)c2)NC(=O)c2c(Cl)cc(Nc3cc(NC(=O)C4CC4)ncn3)c(=O)n21] |
| 53 | 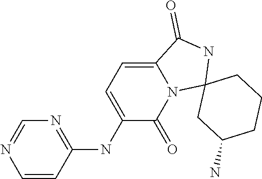 [SMILES: N[C@H]1CCCC2(C1)NC(=O)c1ccc(Nc3ccncn3)c(=O)n12] |
| 54 | 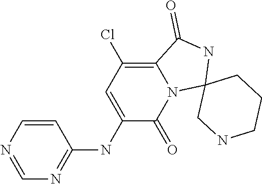 [SMILES: O=C1NC2(CCCNC2)n2c1c(Cl)cc(Nc1ccncn1)c2=O] |
| 55 | 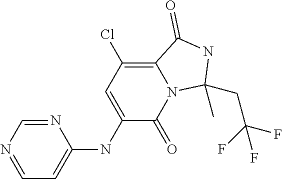 [SMILES: CC1(CC(F)(F)F)NC(=O)c2c(Cl)cc(Nc3ccncn3)c(=O)n21] |
| 56 | 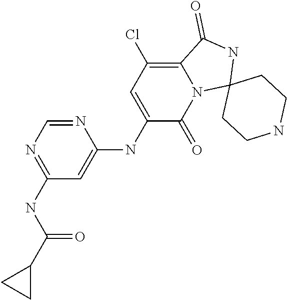 [SMILES: O=C1NC2(CCNCC2)n2c1c(Cl)cc(Nc1cc(NC(=O)C3CC3)ncn1)c2=O] |
| 57 | 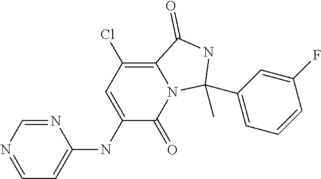 [SMILES: CC1(c2cccc(F)c2)NC(=O)c2c(Cl)cc(Nc3ccncn3)c(=O)n21] |
| 58 | 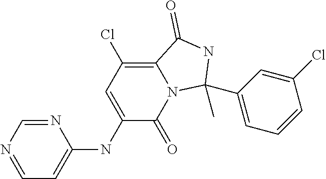 [SMILES: CC1(c2cccc(Cl)c2)NC(=O)c2c(Cl)cc(Nc3ccncn3)c(=O)n21] |
| 59 | 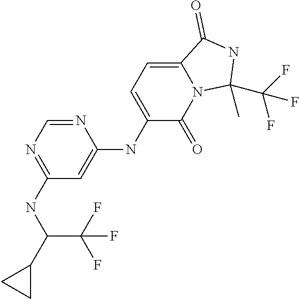 [SMILES: CC1(C(F)(F)F)NC(=O)c2ccc(Nc3cc(NC(C4CC4)C(F)(F)F)ncn3)c(=O)n21] |
| 60 | 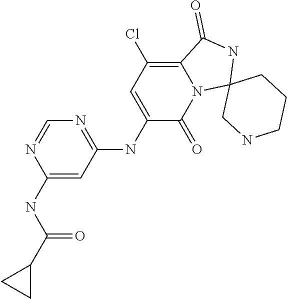 [SMILES: O=C1NC2(CCCNC2)n2c1c(Cl)cc(Nc1cc(NC(=O)C3CC3)ncn1)c2=O] |
| 61 | 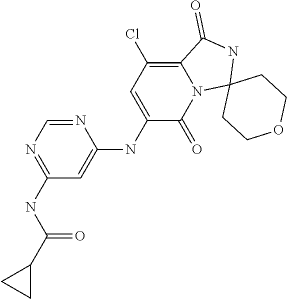 [SMILES: O=C1NC2(CCOCC2)n2c1c(Cl)cc(Nc1cc(NC(=O)C3CC3)ncn1)c2=O] |
| 62 | 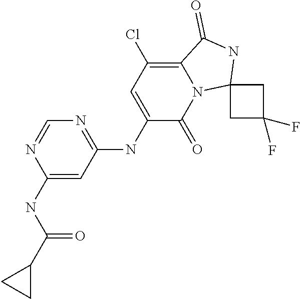 [SMILES: O=C1NC2(CC(F)(F)C2)n2c1c(Cl)cc(Nc1cc(NC(=O)C3CC3)ncn1)c2=O] |
| 63 | 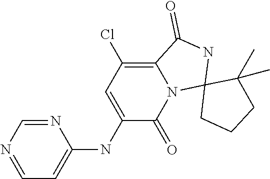 [SMILES: CC1(C)CCCC12NC(=O)c1c(Cl)cc(Nc3ccncn3)c(=O)n12] |
| 64 | 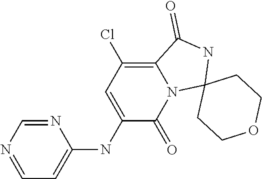 [SMILES: O=C1NC2(CCOCC2)n2c1c(Cl)cc(Nc1ccncn1)c2=O] |
| 65 | 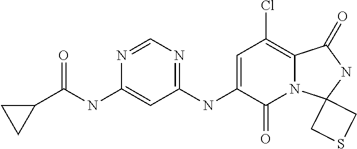 [SMILES: O=C1NC2(CSC2)n2c1c(Cl)cc(Nc1cc(NC(=O)C3CC3)ncn1)c2=O] |
| 66 | 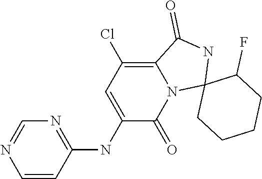 [SMILES: O=C1NC2(CCCCC2F)n2c1c(Cl)cc(Nc1ccncn1)c2=O] |
| 67 | 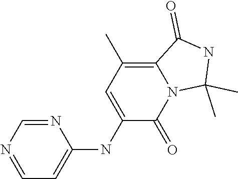 [SMILES: Cc1cc(Nc2ccncn2)c(=O)n2c1C(=O)NC2(C)C] |
| 68 | 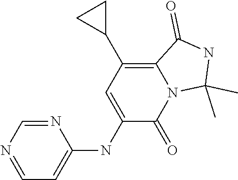 [SMILES: CC1(C)NC(=O)c2c(C3CC3)cc(Nc3ccncn3)c(=O)n21] |
| 69 | 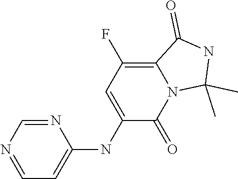 [SMILES: CC1(C)NC(=O)c2c(F)cc(Nc3ccncn3)c(=O)n21] |
| 70 | 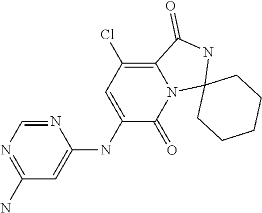 [SMILES: Nc1cc(Nc2cc(Cl)c3n(c2=O)C2(CCCCC2)NC3=O)ncn1] |
| 71 | 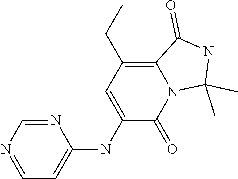 [SMILES: CCc1cc(Nc2ccncn2)c(=O)n2c1C(=O)NC2(C)C] |
| 72 | 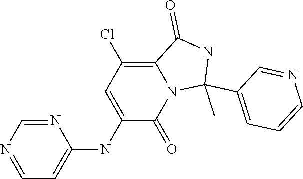 [SMILES: CC1(c2cccnc2)NC(=O)c2c(Cl)cc(Nc3ccncn3)c(=O)n21] |
| 73 | 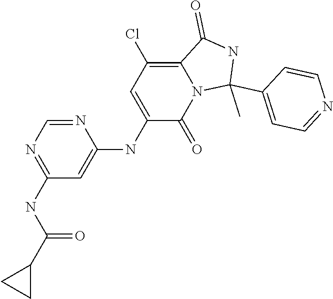 [SMILES: CC1(c2ccncc2)NC(=O)c2c(Cl)cc(Nc3cc(NC(=O)C4CC4)ncn3)c(=O)n21] |
| 74 | 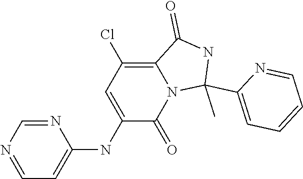 [SMILES: CC1(c2ccccn2)NC(=O)c2c(Cl)cc(Nc3ccncn3)c(=O)n21] |
| 75 | 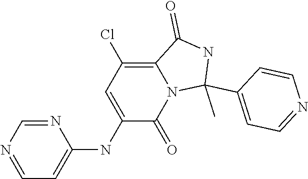 [SMILES: CC1(c2ccncc2)NC(=O)c2c(Cl)cc(Nc3ccncn3)c(=O)n21] |
| 76 | 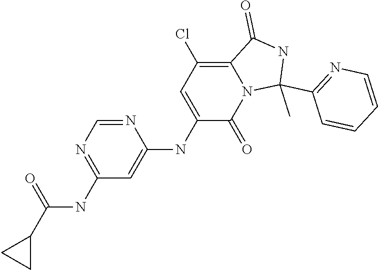 [SMILES: CC1(c2ccccn2)NC(=O)c2c(Cl)cc(Nc3cc(NC(=O)C4CC4)ncn3)c(=O)n21] |
| 77 | 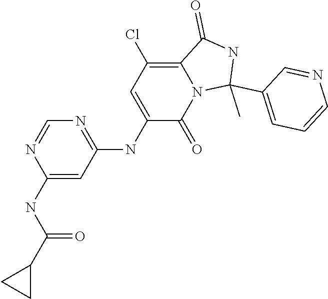 [SMILES: CC1(c2cccnc2)NC(=O)c2c(Cl)cc(Nc3cc(NC(=O)C4CC4)ncn3)c(=O)n21] |
| 78 | 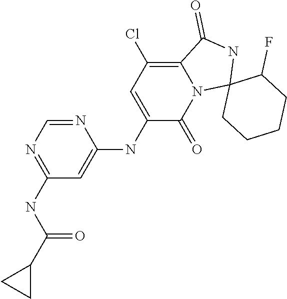 [SMILES: O=C1NC2(CCCCC2F)n2c1c(Cl)cc(Nc1cc(NC(=O)C3CC3)ncn1)c2=O] |
| 79 | 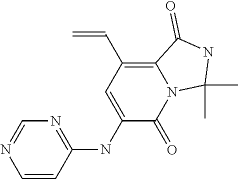 [SMILES: C=Cc1cc(Nc2ccncn2)c(=O)n2c1C(=O)NC2(C)C] |
| 80 | 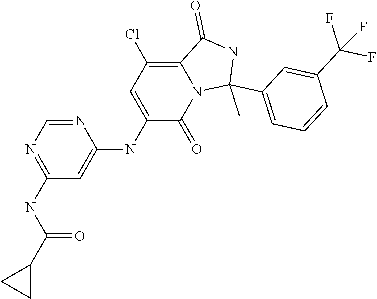 [SMILES: CC1(c2cccc(C(F)(F)F)c2)NC(=O)c2c(Cl)cc(Nc3cc(NC(=O)C4CC4)ncn3)c(=O)n21] |
| 81 | 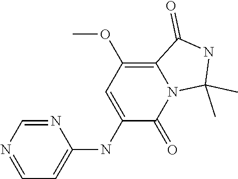 [SMILES: COc1cc(Nc2ccncn2)c(=O)n2c1C(=O)NC2(C)C] |
| 82 | 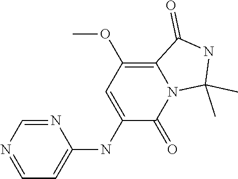 [SMILES: COc1cc(Nc2ccncn2)c(=O)n2c1C(=O)NC2(C)C] |
| 83 | 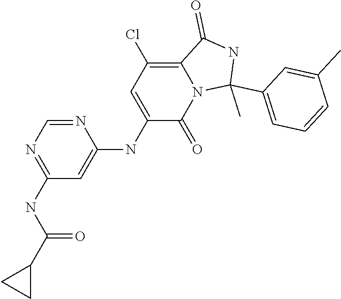 [SMILES: Cc1cccc(C2(C)NC(=O)c3c(Cl)cc(Nc4cc(NC(=O)C5CC5)ncn4)c(=O)n32)c1] |
| 84 | 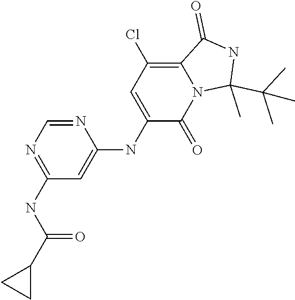 [SMILES: CC(C)(C)C1(C)NC(=O)c2c(Cl)cc(Nc3cc(NC(=O)C4CC4)ncn3)c(=O)n21] |
| 85 | 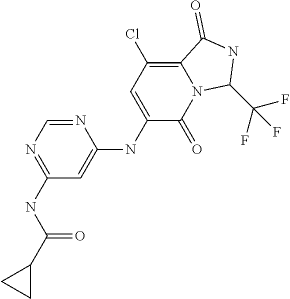 [SMILES: O=C1NC(C(F)(F)F)n2c1c(Cl)cc(Nc1cc(NC(=O)C3CC3)ncn1)c2=O] |
| 86 | 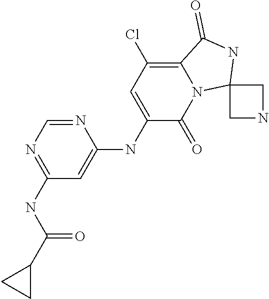 [SMILES: O=C1NC2(CNC2)n2c1c(Cl)cc(Nc1cc(NC(=O)C3CC3)ncn1)c2=O] |
| 87 | 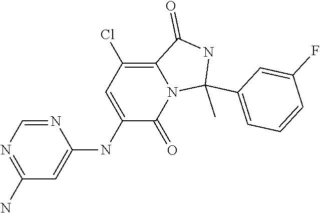 [SMILES: CC1(c2cccc(F)c2)NC(=O)c2c(Cl)cc(Nc3cc(N)ncn3)c(=O)n21] |
| 88 | 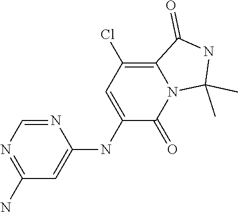 [SMILES: CC1(C)NC(=O)c2c(Cl)cc(Nc3cc(N)ncn3)c(=O)n21] |
| 89 | 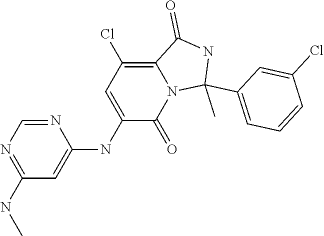 [SMILES: CNc1cc(Nc2cc(Cl)c3n(c2=O)C(C)(c2cccc(Cl)c2)NC3=O)ncn1] |
| 90 | 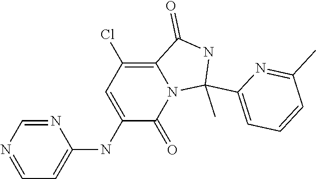 [SMILES: Cc1cccc(C2(C)NC(=O)c3c(Cl)cc(Nc4ccncn4)c(=O)n32)n1] |
| 91 | 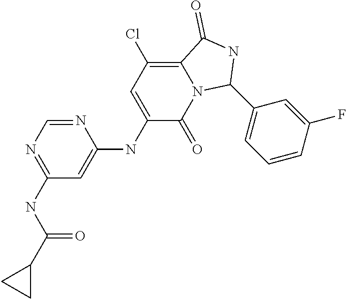 [SMILES: O=C1NC(c2cccc(F)c2)n2c1c(Cl)cc(Nc1cc(NC(=O)C3CC3)ncn1)c2=O] |
| 92 | 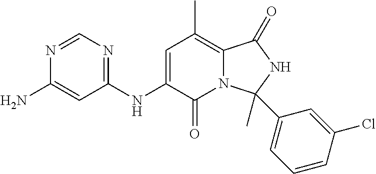 [SMILES: Cc1cc(Nc2cc(N)ncn2)c(=O)n2c1C(=O)NC2(C)c1cccc(Cl)c1] |
| 93 | 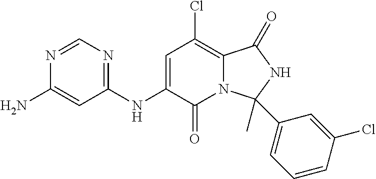 [SMILES: CC1(c2cccc(Cl)c2)NC(=O)c2c(Cl)cc(Nc3cc(N)ncn3)c(=O)n21] |
| 94 | 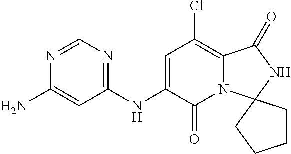 [SMILES: Nc1cc(Nc2cc(Cl)c3n(c2=O)C2(CCCC2)NC3=O)ncn1] |
| 95 | 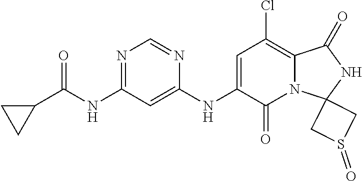 [SMILES: O=C1NC2(CS(=O)C2)n2c1c(Cl)cc(Nc1cc(NC(=O)C3CC3)ncn1)c2=O] |
| 96 | 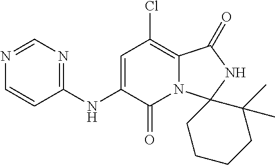 [SMILES: CC1(C)CCCCC12NC(=O)c1c(Cl)cc(Nc3ccncn3)c(=O)n12] |
| 97 | 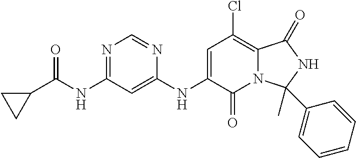 [SMILES: CC1(c2ccccc2)NC(=O)c2c(Cl)cc(Nc3cc(NC(=O)C4CC4)ncn3)c(=O)n21] |
| 98 | 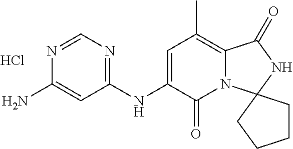 [SMILES: Cc1cc(Nc2cc(N)ncn2)c(=O)n2c1C(=O)NC21CCCC1 ; Cl] |
| 99 | 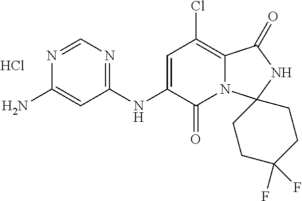 [SMILES: Cl ; Nc1cc(Nc2cc(Cl)c3n(c2=O)C2(CCC(F)(F)CC2)NC3=O)ncn1] |
| 100 | 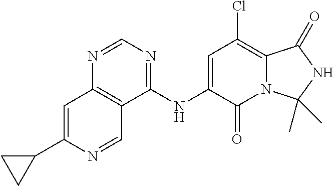 [SMILES: CC1(C)NC(=O)c2c(Cl)cc(Nc3ncnc4cc(C5CC5)ncc34)c(=O)n21] |
| 101 | 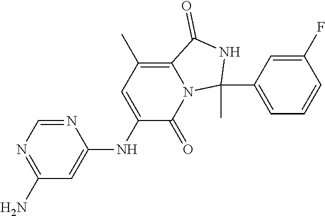 [SMILES: Cc1cc(Nc2cc(N)ncn2)c(=O)n2c1C(=O)NC2(C)c1cccc(F)c1] |
| 102 | 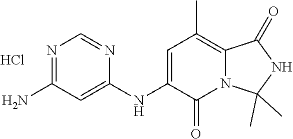 [SMILES: Cc1cc(Nc2cc(N)ncn2)c(=O)n2c1C(=O)NC2(C)C ; Cl] |
| 103 | 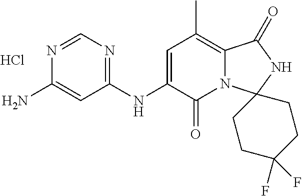 [SMILES: Cc1cc(Nc2cc(N)ncn2)c(=O)n2c1C(=O)NC21CCC(F)(F)CC1 ; Cl] |
| 104 | 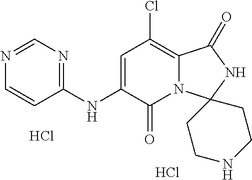 [SMILES: O=C1NC2(CCNCC2)n2c1c(Cl)cc(Nc1ccncn1)c2=O ; Cl ; Cl] |
| 105 | 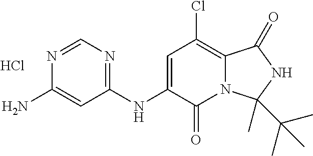 [SMILES: CC(C)(C)C1(C)NC(=O)c2c(Cl)cc(Nc3cc(N)ncn3)c(=O)n21 ; Cl] |
| 106 | 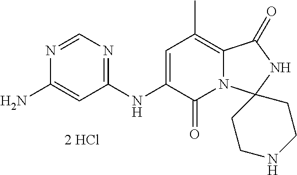 [SMILES: Cc1cc(Nc2cc(N)ncn2)c(=O)n2c1C(=O)NC21CCNCC1 ; "2 HCl"] |
| 107 | 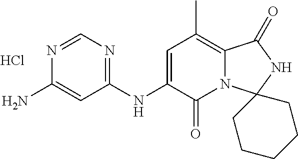 [SMILES: Cc1cc(Nc2cc(N)ncn2)c(=O)n2c1C(=O)NC21CCCCC1 ; Cl] |
| 108 | 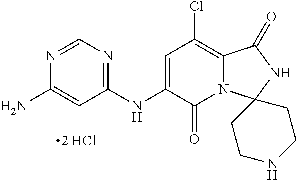 [SMILES: Nc1cc(Nc2cc(Cl)c3n(c2=O)C2(CCNCC2)NC3=O)ncn1 ; "·2 HCl"] |
| 109 | 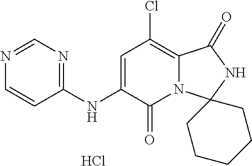 [SMILES: O=C1NC2(CCCCC2)n2c1c(Cl)cc(Nc1ccncn1)c2=O ; Cl] |
| 110 | 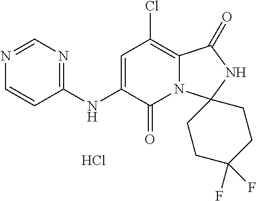 [SMILES: O=C1NC2(CCC(F)(F)CC2)n2c1c(Cl)cc(Nc1ccncn1)c2=O ; Cl] |
| 111 | 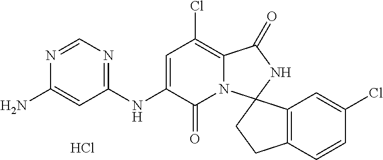 [SMILES: Nc1cc(Nc2cc(Cl)c3n(c2=O)C2(CCc4ccc(Cl)cc42)NC3=O)ncn1 ; Cl] |
| 112 | 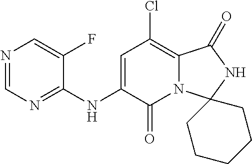 [SMILES: O=C1NC2(CCCCC2)n2c1c(Cl)cc(Nc1ncncc1F)c2=O] |

Other examples of MM(inhibitors that may be used according to any of the methods described herein include cercosporamide; SEL201; CGP57380 (see, Knauf et al., Mol. Cell. Biol. 21:5500-5511, 2001); CGP52088 (see Tschopp et al., Mol. Cell. Biol. Res. Commun. 3:205-211, 2000); YYC-37 (Schmid, “Targeting cap-dependent translation for cancer therapy: Identification of novel Mnk kinase inhibitors with enzymatic assays,” www.fhnw.ch/lifesciences/master/master-thesis/MS_MT_Schmid_Raffaela_2014.pdf, 2014); a retinamide retinonic acid metabolism blocking agent (also known as retinamide RAIVIBA) (e.g., VNLG-152) (see, PCT Publication No. WO 2010/036404; Ramalingam et al., Oncotarget 5:530-543, 2014; Mbatia et al., J. Med. Chem. 58:1900-1914, 2015); a sulfoximine substituted quinazoline derivative, as disclosed in U.S. Pat. No. 8,901,138; a pyrrolopyrimidine compound as disclosed in U.S. Pat. No. 8,697,713, PCT Publication No. WO 2013/174743, or PCT Publication No. WO 2014/044691; a thienopyrimidine compound as disclosed in U.S. Pat. No. 8,486,953, U.S. Patent Publication No. US 2010/0143341, PCT Publication No. WO 2013/174744; or PCT Publication No. WO 2014/118229; a piperazine-based compound (e.g., ETC036 or ETC037) as disclosed in PCT Publication No. WO 2014/088519; a bicyclic heterocyclic derivative (e.g., compound 20, 359, or 416) as disclosed in PCT Publication No. WO 2013/147711; a pyrazolopyrimidine compound as disclosed in U.S. Pat. No. 8,071,607; a substituted thiazolopyrimidine compound as disclosed in PCT Publication No. WO 2014/135480; a substituted imidazopyridazine compound as disclosed in U.S. Patent Publication Nos. US 2014/0296231; US 2014/0288069; US 2014/0228370; US 2014/0194430; PCT Publication Nos. WO 2013/149909; WO 2013/144189, WO 2013/087581, WO 2014/128093, WO 2014/076162, or WO 2014/118135; a substituted pyrazolopyrimidinylamino-indazole compound as disclosed in PCT Publication No. WO 2014/118226; a substituted indazol-pyrrolopyrimidine compound as disclosed in PCT Publication No. WO 2014/048894 or WO 2014/048869; a substituted benzothienopyrimidine compound as disclosed in PCT Publication No. WO 2013/174735; sulfoximine substituted quinazoline compound as disclosed in PCT Publication No. WO 2014206922; or a heterocyclyl aminoimidazopyridazine compound as disclosed in PCT Publication No. WO 2012/175591 (each of the compounds of these references is incorporated herein by reference, in their entirety).

In certain embodiments, a MNK inhibitor is a specific MNK inhibitor of any one of Formulae I, Ia, IIa, IIb, IIIa, IIIb, IVa, IVb, Va, Vb VI, VIIa and VIIb, or from Table 1 or Table 2, which is formulated as a pharmaceutical composition in an amount effective to treat a particular disease or condition of interest (e.g., cancer, chronic infection) upon administration of the pharmaceutical composition to a mammal (e.g., human). In particular embodiments, a pharmaceutical composition comprises a MNK inhibitor as described herein and a pharmaceutically acceptable carrier, diluent or excipient.

In this regard, a “pharmaceutically acceptable carrier, diluent or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier that has been approved by the United States Food and Drug Administration as being acceptable for use in humans or domestic animals.

Further, a “mammal” includes primates, such as humans, monkeys and apes, and non-primates such as domestic animals, including laboratory animals and household pets (e.g., cats, dogs, swine, cattle, sheep, goats, horses, rabbits), and non-domestic animals, such as wildlife or the like.

A pharmaceutical composition of this disclosure can be prepared by combining or formulating a MNK inhibitor as described herein with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Exemplary routes of administering such pharmaceutical compositions include oral, topical, transdermal, inhalation, parenteral, sublingual, buccal, rectal, vaginal, and intranasal. The term parenteral, as used herein, includes subcutaneous injections, intravenous, intramuscular, intrasternal injection or infusion techniques. Pharmaceutical compositions of this disclosure are formulated to allow the active ingredients contained therein to be bioavailable upon administration to a patient. Compositions that will be administered to a subject or patient take the form of one or more dosage units, where, for example, a tablet may be a single dosage unit, and a container of a MNK inhibitor as described herein in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). A composition to be administered will, in any event, contain a therapeutically effective amount of a MNK inhibitor of this disclosure, or a pharmaceutically acceptable salt thereof, for modulating an immune response to aid in treatment of a disease or condition of interest in accordance with the teachings herein.

A pharmaceutical composition of a MNK inhibitor as described herein may be in the form of a solid or liquid. In one aspect, the carrier(s) are particulate so that the compositions are, for example, in tablet or powder form. The carrier(s) may be liquid, with a composition being, for example, an oral syrup, injectable liquid or an aerosol, which is useful in, for example, inhalatory administration. When intended for oral administration, a pharmaceutical composition of a MNK inhibitor of this disclosure is preferably in either solid or liquid form, where semi-solid, semi-liquid, suspension and gel forms are included within the forms considered herein as either solid or liquid.

As a solid composition for oral administration, a pharmaceutical composition of a MNK inhibitor as described herein may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

A pharmaceutical composition may be in the form of a liquid, for example, an elixir, syrup, solution, emulsion or suspension. The liquid may be for oral administration or for delivery by injection, as two examples. When intended for oral administration, preferred compositions contain, in addition to a MNK inhibitor, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of MNK inhibitors, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of a MM(inhibitor intended for either parenteral or oral administration should contain an amount of a MM(inhibitor of this disclosure such that a suitable dosage will be obtained.

A pharmaceutical composition of a MNK inhibitor may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, a composition of a MNK inhibitor of this disclosure may be included with a transdermal patch or iontophoresis device.

The pharmaceutical composition of a MNK inhibitor may be intended for rectal administration, in the form, for example, of a suppository, which will melt in the rectum and release the drug. A composition for rectal administration may contain an oleaginous base as a suitable nonirritating excipient. Such bases include, for example, lanolin, cocoa butter or polyethylene glycol.

The pharmaceutical composition of a MNK inhibitor may include various materials that modify the physical form of a solid or liquid dosage unit. For example, the composition may include materials that form a coating shell around the active ingredients.

The materials that form the coating shell are typically inert, and may be selected from, for example, sugar, shellac, and other enteric coating agents. Alternatively, the active ingredients may be encased in a gelatin capsule.

The pharmaceutical composition of this disclosure in solid or liquid form may include an agent that binds to a MM(inhibitor described herein and thereby assist in the delivery of the compound. Suitable agents that may act in this capacity include a monoclonal or polyclonal antibody, a protein or a liposome.

A pharmaceutical composition of a MNK inhibitor may consist of dosage units that can be administered as an aerosol. The term aerosol is used to denote a variety of systems ranging from those of colloidal nature to systems consisting of pressurized packages.

Delivery may be by a liquefied or compressed gas or by a suitable pump system that dispenses the active ingredients. Aerosols of MNK inhibitors may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation, may determine preferred aerosol formulations and delivery modes.

A pharmaceutical composition of this disclosure may be prepared by methodology well-known in the pharmaceutical art. For example, a pharmaceutical composition intended to be administered by injection can be prepared by combining a MNK inhibitor as described herein with a sterile solvent so as to form a solution. A surfactant may be added to facilitate the formation of a homogeneous solution or suspension. Surfactants are compounds that non-covalently interact with a compound of this disclosure so as to facilitate dissolution or homogeneous suspension of the compound in an aqueous delivery system.

### Hyperproliferative Disease

In one aspect, the present disclosure provides a method of assessing whether a human subject having a hyperproliferative disease is likely to respond to treatment with a MNK inhibitor, comprising measuring a first translational rate, first translational efficiency, first mRNA level or any combination thereof of one to about 100 genes as set forth in any of Tables 3-6, 9, 10 and 12 in a sample from the subject prior to contacting the sample with a MNK inhibitor; measuring a second translational rate, second translational efficiency, second mRNA level or any combination thereof of one to about 100 genes as set forth in any of Tables 3-6, 9, 10 and 12 in a sample from the subject after contacting the sample with the MNK inhibitor; and identifying the subject as likely to respond to treatment with the MNK inhibitor when the first translational rate, first translational efficiency, first mRNA level or any combination thereof of the one to about 100 genes as set forth in any of Tables 3-6, 9, 10 and 12 differs (e.g., 0.75 log₂, 1.0 log₂ or 2.0 log₂) from the second translational rate, second translational efficiency, second mRNA level or any combination thereof of the one to about 100 genes as set forth in any of Tables 3-6, 9, 10 and 12. In certain embodiments, the present disclosure provides a method for reducing the risk of developing a hyperproliferative disease, comprising: administering to a subject at risk of developing a hyperproliferative disease a therapeutically effective amount of a MNK inhibitor that alters the translational rate, translational efficiency, mRNA level or any combination thereof of any one or more of the genes (including any alleles, homologs, or orthologs) listed in any of Tables 3-6, 9, 10 and 12.

In certain embodiments, treatment with a MNK inhibitor of this disclosure results in regulation of genes containing a consensus sequence(s), such as a 5′-UTR, 3′UTR, or both as provided in Tables 8 and 11. For example, regulation includes inhibition of translation initiation, control of mRNA stability, or control of transcription. Components that may affect regulation include translation factors (e.g., eIF4E) and RNA binding proteins, (e.g., hnRNPA1). In certain embodiments, such MNK inhibitor regulation can be useful in determining the sensitivity of a disease, or a subject in need to MNK inhibition and in determining response of a subject to MNK inhibition.

In other aspects, the present disclosure provides a method for treating a hyperproliferative disease in a human subject, comprising administering an effective amount of a MNK inhibitor to a subject having or suspected of having a hyperproliferative disease when a sample obtained from the subject and prior to contacting the sample with a MNK inhibitor has a translational rate, translational efficiency, mRNA level or any combination thereof of one to about 100 genes as set forth in any of Tables 3-6, 9, 10 and 12 above or below a translational rate, translational efficiency, mRNA level or any combination thereof of one to about 100 genes as set forth in any of Tables 3-6, 9, 10 and 12 in the sample contacted with the MNK inhibitor.

In still other aspects, the present disclosure provides a method of identifying a human subject as a candidate for treating a hyperproliferative disease with a MNK inhibitor, comprising (a) determining a first translational rate, first translational efficiency, mRNA level or any combination thereof of one to about 100 genes as set forth in any of Tables 3-6, 9, 10 and 12 in a sample from a subject having or suspected of having a hyperproliferative disease; (b) determining a second translational rate, second translational efficiency, mRNA level or any combination thereof of one to about 100 genes as set forth in any of Tables 3-6, 9, 10 and 12 in a control sample, wherein the control sample is from a subject known to respond to the MNK inhibitor and wherein the sample has not been contacted with the MNK inhibitor; and (c) identifying the subject as a candidate for treating hyperproliferative disease with the MNK inhibitor when the first translational rate, first translational efficiency, first mRNA level or any combination thereof of the one to about 100 genes as set forth in any of Tables 3-6, 9, 10 and 12 of step (a) is comparable to the second translational rate, second translational efficiency, second mRNA level or any combination thereof of the one to about 100 genes as set forth in any of Tables 3-6, 9, 10 and 12 of step (b).

In another aspect, the instant disclosure provides a method for selecting a therapy for a particular human subject in a population of subjects being considered for therapy, comprising (a) determining a translational rate, translational efficiency, mRNA level or any combination thereof of one to about 100 genes as set forth in any of Tables 3-6, 9, 10 and 12 in a sample from a subject having or suspected of having a hyperproliferative disease prior to contacting the subject sample with a MNK inhibitor; and (b) comparing the translational rate, translational efficiency, mRNA level or any combination thereof of the one to about 100 genes as set forth in any of Tables 3-6, 9, 10 and 12 in the subject sample to a translational rate, translational efficiency, mRNA level or any combination thereof of the one to about 100 genes as set forth in any of Tables 3-6, 9, 10 and 12 in a control sample, wherein a change in the translational rate, translational efficiency, mRNA level or any combination thereof of the one to about 100 genes as set forth in any of Tables 3-6, 9, 10 and 12 in the subject sample relative to the control sample identifies the subject as one who is likely to respond to treatment with the MNK inhibitor; wherein a therapy comprising the MNK inhibitor is selected or recommended if the subject having or suspected of having a hyperproliferative disease is identified as likely to respond to treatment with the MNK inhibitor; or wherein a therapy comprising the MNK inhibitor is not selected or recommended if the subject is not identified as likely to respond to treatment with a the MNK inhibitor.

In still another aspect, the instant disclosure provides a method of maximizing therapeutic efficacy of a MNK inhibitor for a human subject having a hyperproliferative disease, comprising (a) detecting a translational rate, translational efficiency, mRNA level or any combination thereof of one to about 100 genes as set forth in any of Tables 3-6, 9, 10 and 12 in a sample obtained from the subject prior to any administration of a MNK inhibitor to the subject; (b) comparing the translational rate, translational efficiency, mRNA level or any combination thereof of the one to about 100 genes as set forth in any of Tables 3-6, 9, 10 and 12 in the subject sample to a translational rate, translational efficiency, mRNA level or any combination thereof of the one to about 100 genes as set forth in any of Tables 3-6, 9, 10 and 12 in a control sample, wherein a change in the translational rate, translational efficiency, mRNA level or any combination thereof of the one to about 100 genes as set forth in any of Tables 3-6, 9, 10 and 12 in the subject sample relative to the control sample identifies the subject as one who is likely to respond to treatment with the MNK inhibitor; and (c) determining that treating with an effective amount of a MNK inhibitor will maximize efficacy of the treatment for the subject.

In certain aspects, the instant disclosure provides a method of monitoring response of a human subject having a hyperproliferative disease to treatment with a MNK inhibitor, comprising (a) determining that a sample obtained from the subject treated with a MMK inhibitor has a translational rate, translational efficiency, mRNA level or any combination thereof of one to about 100 genes as set forth in any of Tables 3-6, 9, 10 and 12 above or below the level of a control sample of the one to about 100 genes as set forth in any of Tables 3-6, 9, 10 and 12; and (b) determining that the treatment for the subject comprises an effective amount of a MM(inhibitor.

In yet another aspect, the instant disclosure provides a method of identifying a biomarker for determining responsiveness to a MM(inhibitor, comprising (a) measuring a translational rate, translational efficiency, mRNA level or any combination thereof of one to about 100 candidate biomarkers as set forth in any of Tables 3-6, 9, 10 and 12 in a sample from the subject prior to contacting the sample with a MNK inhibitor; and (b) comparing the translational rate, translational efficiency, mRNA level or any combination thereof of the one to about 100 candidate biomarkers as set forth in any of Tables 3-6, 9, 10 and 12 in the subject sample to a translational rate, translational efficiency, mRNA level or any combination thereof of the one to about 100 candidate biomarkers as set forth in any of Tables 3-6, 9, 10 and 12 in a control sample, wherein a change in the translational rate, translational efficiency, mRNA level or any combination thereof of the one to about 100 candidate biomarkers as set forth in any of Tables 3-6, 9, 10 and 12 in the subject sample relative to the control sample identifies the subject as one who is likely to respond to treatment with the MNK inhibitor.

Genes having an altered translational rate, translational efficiency, mRNA level or any combination thereof due to a MNK inhibitor can be used as biomarkers for hyperproliferative disease as described herein. MNK inhibitor biomarkers may include one to all of the genes identified in any of Tables 3-6, 9, 10 and 12. In certain embodiments, a MNK inhibitor biomarker comprises one gene, two genes, five genes, ten genes, 15 genes, 20 genes, 25 genes, 30 genes, 35 genes, 40 genes, 45 genes, 50 genes, 55 genes, 60 genes, 65 genes, 70 genes, 75 genes, 80 genes, 85 genes, 90 genes, 95 genes, 100 genes, 105 genes, 110 genes, 115 genes, or 120 genes. In further embodiments, a MNK inhibitor biomarker comprises from one gene to about 100 genes, from one gene to about 75 genes, from one gene to about 50 genes, from one gene to about 25 genes, from one gene to about ten genes, from one gene to about five genes, from two gene to about eight genes, or from three gene to about six genes.

In further aspects, the instant disclosure provides a method for diagnosing a hyperproliferative disease in a human subject that would be responsive to a MNK inhibitor, comprising (a) measuring a translational rate, translational efficiency, mRNA level or any combination thereof of one to about 100 candidate biomarkers as set forth in any of Tables 3-6, 9, 10 and 12 in a sample from the subject prior to contacting the sample with a MNK inhibitor; and (b) comparing the translational rate, translational efficiency, mRNA level or any combination thereof of the one to about 100 candidate biomarkers as set forth in any of Tables 3-6, 9, 10 and 12 in the subject sample to a translational rate, translational efficiency, mRNA level or any combination thereof of the one to about 100 candidate biomarkers as set forth in any of Tables 3-6, 9, 10 and 12 in a control sample; wherein a change in the translational rate, translational efficiency, mRNA level or any combination thereof of the one to about 100 candidate biomarkers as set forth in any of Tables 3-6, 9, 10 and 12 in the subject sample relative to the control sample diagnoses the subject as one who has a hyperproliferative disease that is likely to respond to treatment with the MNK inhibitor.

In still further embodiments, the instant disclosure provides a method of determining a prognosis of a human subject having a hyperproliferative disease if treated with a MNK inhibitor, comprising (a) determining the translational rate, translational efficiency, mRNA level or any combination thereof of the one to about 100 candidate biomarkers as set forth in any of Tables 3-6, 9, 10 and 12 in a sample from the subject prior to contacting the sample with a MNK inhibitor; (b) comparing the translational rate, translational efficiency, mRNA level or any combination thereof of the one to about 100 candidate biomarkers as set forth in any of Tables 3-6, 9, 10 and 12 in the subject sample to a translational rate, translational efficiency, mRNA level or any combination thereof of the one to about 100 candidate biomarkers as set forth in any of Tables 3-6, 9, 10 and 12 in a control sample; wherein the subject is classified as having a good prognosis if the subject is treated with an effective amount of a MM(inhibitor.

In additional aspects, the instant disclosure provides a kit for determining whether a human subject having a hyperproliferative disease may benefit from treatment with a MNK inhibitor, comprising (a) reagents useful for determining the translational rate, translational efficiency, mRNA level or any combination thereof of the one to about 100 candidate biomarkers as set forth in any of Tables 3-6, 9, 10 and 12 in a sample from the subject prior to contacting the sample with a MNK inhibitor; and; (b) instructions for use of the reagents to determine the translational rate, translational efficiency, mRNA level or any combination thereof of the one to about 100 candidate biomarkers as set forth in any of Tables 3-6, 9, 10 and 12 in a sample from the subject and a control sample prior to contacting the sample with a MM(inhibitor, wherein a change in translational rate, translational efficiency, mRNA level or any combination thereof of the one to about 100 candidate biomarkers as set forth in any of Tables 3-6, 9, 10 and 12 relative to a control sample indicates that the subject may benefit from treatment with a MNK inhibitor.

In any of the aforementioned embodiments, a gene having an altered translational rate, translational efficiency, mRNA level or any combination thereof, or a biomarker comprises any gene found in any of Tables 3-6, 9, 10 and 12, such as NR2F1, VLDLR, C2CD2L, BCL9L, CAV2, ACCN2, FZD5, RBKS, ULK2, KLF5, KLF9, SYT4, TMSB4Y, SKI, CENPBD1, LPAR5, ST3GAL1, WNT8A, WASF1, B3GNT7, TNFRSF14, VANGL2, ZNF771, RPS6KL1, ZNF425, CCDC85C, PER3, RASGRF1, EDN1, FLT3LG, SLC35A2, NR4A3, GLIPR2, ARMC7, PPP1R3D, PSRC1, KIAA0748, SETD1B, SLC16A3, MOB3C, LHFPL2, TTLL11, PCDH9, STMN3, FAM212B, C6orf225, SMN2 or any combination thereof.

In any of the of the aforementioned aspects or embodiments, any one or more of the genes as set forth in any of Tables 3-6, 9, 10 and 12 having their translational rate, translational efficiency, mRNA level or any combination thereof altered by the MNK inhibitor may contain a 5′-UTR recognition sequence of Table 8, a 3′-UTR recognition sequence of Table 11, or a combination thereof. In certain embodiments, the 5′-UTR recognition or 3′-UTR recognition sequence can present or occur more than once, such as one to about 15 times, one to about 10 times, or one to about 5 times. In certain embodiments, a 3′-UTR recognition sequence is involved in mRNA stability.

In certain embodiments, combinations of therapies for use in the methods described herein comprise (1) a MNK inhibitor and a modulator of an eIF4A, (2) a MNK inhibitor and a modulator of an eIF4E, (3) a MNK inhibitor and a modulator of an eIF5A, or (6) any combination thereof In further embodiments, a MNK inhibitor can be used in combination with an adjunctive therapy, such as an anti-cancer agent.

Anti-cancer agents include chemotherapeutic drugs. A chemotherapeutic agent includes, for example, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), or a DNA repair inhibitor. In further embodiments, a MNK inhibitor is used in combination with a chemotherapeutic agent and a PD-1 specific antibody or binding fragment thereof In still further embodiments, a MNK inhibitor is used in combination with a chemotherapeutic agent and a PD-Ll specific antibody or binding fragment thereof. In yet further embodiments, a MNK inhibitor is used in combination with a chemotherapeutic agent and a CTLA4 specific antibody or binding fragment thereof, or fusion protein. In yet further embodiments, a MNK inhibitor is used in combination with a chemotherapeutic agent and a LAG3 specific antibody or binding fragment thereof, or fusion protein.

Chemotherapeutic agents include, for example, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (methotrexate, pemetrexed, mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones, eribulin and navelbine; epidipodophyllotoxins (etoposide, teniposide); DNA damaging agents (actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, temozolamide, teniposide, triethylenethiophosphoramide and etoposide (VP 16)); DNA methyltransferase inhibitors (azacytidine); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkylsulfonates (busulfan), nitrosoureas (carmustine (BCNU) and analogs, streptozocin), triazenes (dacarbazine (DTIC)); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein, pomalidomide) and growth factor inhibitors (vascular endothelial growth factor (VEGF) inhibitors, such as ziv-aflibercept; fibroblast growth factor (FGF) inhibitors); inhibitors of apoptosis protein (IAP) antagonists (birinapant); histone deacetylase (HDAC) inhibitors (vorinostat, romidepsin, chidamide, panobinostat, mocetinostat, abexinostat, belinostat, entinostat, resminostat, givinostat, quisinostat, SB939); proteasome inhibitors (ixazomib); angiotensin receptor blocker; nitric oxide donors; anti-sense oligonucleotides; antibodies (trastuzumab, panitumumab, pertuzumab, cetuximab, adalimumab, golimumab, infliximab, rituximab, ocrelizumab, ofatumumab, obinutuzumab, alemtuzumab, abciximab, atlizumab, daclizumab, denosumab, efalizumab, elotuzumab, rovelizumab, ruplizumab, ustekinumab, visilizumab, gemtuzumab ozogamicin, brentuximb vedotin); chimeric antigen receptors; cell cycle inhibitors (flavopiridol, roscovitine, bryostatin-1) and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); PARP inhibitors (niraparib, olaparib); focal adhesion kinase (FAK) inhibitors (defactinib (VS-6063), VS-4718, VS-6062, GSK2256098); growth factor signal transduction kinase inhibitors (cediranib, galunisertib, rociletinib, vandetanib, afatinib, EGF816, AZD4547); c-Met inhibitors (capmatinib, INC280); ALK inhibitors (ceritinib, crizotinib); mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors.

In certain embodiments, a chemotherapeutic is a B-Raf inhibitor, a MEK inhibitor, a VEGF inhibitor, a VEGFR inhibitor, a tyrosine kinase inhibitor, an anti-mitotic agent, or any combination thereof In a specific embodiment, the chemotherapeutic is vemurafenib, dabrafenib, trametinib, cobimetinib, sunitinib, erlotinib, paclitaxel, docetaxel, or any combination thereof.

In certain embodiments, a therapy that induces or enhances an anti-cancer response, for example, a vaccine, an inhibitor of an immunosuppression signal, a B-Raf inhibitor, a MEK inhibitor, a VEGF inhibitor, a VEGFR inhibitor, a tyrosine kinase inhibitor, a cytotoxic agent, a chemotherapeutic, or any combination thereof, is used in combination with a MNK inhibitor in the immune modulation methods described herein, wherein the therapy that induces or enhances an anti-cancer response does not antagonize, reduce, diminish, or decrease the inhibitory activity of a MNK inhibitor on one or more inhibitory immune checkpoint molecules. An antagonistic combination with a MNK inhibitor may be ascertained by measuring translational rate, translational efficiency, mRNA levels or any combination thereod (e.g., as described in Example 1 herein) as a readout of the inhibitory activity of a MNK inhibitor, with and without the therapy that induces or enhances anti-cancer response. In certain embodiments, a combination of a MNK inhibitor and a therapy that induces or enhances anti-cancer response will not antagonize the inhibitory activity of the MNK inhibitor or will only decrease the inhibitory activity of the MNK inhibitor by less than 25%, 20%, 15%, 10%, 5%, 2%, 1%, 0.5%, 0.25%, or 0.1%.

In any of the combination therapies described herein, a combination of a MNK inhibitor and another therapy or modulator can be administered serially, simultaneously, or concurrently. When administering serially, a MNK inhibitor or pharmaceutical composition thereof is formulated in a separate composition from a second (or third, etc.) therapy, modulator or pharmaceutical compositions thereof. When administering simultaneously or concurrently, a first and second (or third, etc.) therapy or modulator may be formulated in separate compositions or formulated in a single composition. In any of these embodiments, the single or combination therapies can be administered as a single dose unit or administered as a single dose unit a plurality of times (daily, weekly, biweekly, monthly, biannually, annually, etc., or any combination thereof).

In certain embodiments, a combination therapy described herein is used in a method for treating a hyperproliferative disease. As used herein, “hyperproliferative disorder” or “hyperproliferative disease” refers to excessive growth or proliferation as compared to a normal cell or an undiseased cell. Exemplary hyperproliferative disorders include dysplasia, neoplasia, non-contact inhibited or oncogenically transformed cells, tumors, cancers, carcinoma, sarcoma, malignant cells, pre-malignant cells, as well as non-neoplastic or non-malignant hyperproliferative disorders (e.g., adenoma, fibroma, lipoma, leiomyoma, hemangioma, fibrosis, restenosis, or the like). In certain embodiments, a cancer being treated by immune modulation via compositions and methods of this disclosure includes carcinoma (epithelial), sarcoma (connective tissue), lymphoma or leukemia (hematopoietic cells), germ cell tumor (pluripotent cells), blastoma (immature “precursor” cells or embryonic tissue), or any combination thereof. These various forms of hyperproliferative disease are known in the art and have established criteria for diagnosis and classification (e.g., Hanahan and Weinberg, Cell 144:646, 2011; Hanahan and Weinberg Cell 100:57, 2000; Cavallo et al., Canc. Immunol. Immunother. 60:319, 2011; Kyrigideis et al., J. Carcinog. 9:3, 2010). In certain embodiments, a hyperproliferative disease may comprise an autoimmune and inflammatory disease.

A wide variety of hyperproliferative disorders, including solid tumors and leukemias, are amenable to the MNK inhibitor compositions and methods disclosed herein. Exemplary cancers that may be treated by immune modulation of this disclosure include adenocarcinoma of the breast, prostate, and colon; all forms of bronchogenic carcinoma of the lung; myeloid; melanoma; hepatoma; neuroblastoma; papilloma; apudoma; choristoma;

branchioma; malignant carcinoid syndrome; carcinoid heart disease; and carcinoma (e.g., Walker, basal cell, basosquamous, Brown-Pearce, ductal, Ehrlich tumor, Krebs 2, merkel cell, mucinous, non-small cell lung, oat cell, papillary, scirrhous, bronchiolar, bronchogenic, squamous cell, and transitional cell). Additional representative cancers that may be treated include histiocytic disorders; histiocytosis malignant; immunoproliferative small intestinal disease; plasmacytoma; reticuloendotheliosis; melanoma; chondroblastoma; chondroma; chondrosarcoma; fibroma; fibrosarcoma; giant cell tumors; histiocytoma; lipoma; liposarcoma; mesothelioma; myxoma; myxosarcoma; osteoma; osteosarcoma; chordoma; craniopharyngioma; dysgerminoma; hamartoma; mesenchymoma; mesonephroma; myosarcoma; ameloblastoma; cementoma; odontoma; teratoma; thymoma; and trophoblastic tumor.

Exemplary hematological malignancies include acute lymphoblastic leukemia (ALL), acute myeloid leukemia (AML), chronic myelogenous leukemia (CML), chronic eosinophilic leukemia (CEL), myelodysplastic syndrome (MDS), Hodgkin's lymphoma, non-Hodgkin's lymphoma (NHL) (e.g., follicular lymphoma, diffuse large B-cell lymphoma, or chronic lymphocytic leukemia), or multiple myeloma (MM).

Still further exemplary hyperproliferative disorders include adenoma; cholangioma; cholesteatoma; cyclindroma; cystadenocarcinoma; cystadenoma; granulosa cell tumor; gynandroblastoma; hepatoma; hidradenoma; islet cell tumor; Leydig cell tumor; sertoli cell tumor; thecoma; leimyoma; leiomyosarcoma; myoblastoma; myomma; myosarcoma; rhabdomyoma; rhabdomyosarcoma; ependymoma; ganglioneuroma; glioma; medulloblastoma; meningioma; neurilemmoma; neuroblastoma; neuroepithelioma; neurofibroma; neuroma; paraganglioma; paraganglioma nonchromaffin; angiokeratoma; angiolymphoid hyperplasia with eosinophilia; angioma sclerosing; angiomatosis; glomangioma; hemangioendothelioma; hemangioma; hemangiopericytoma; hemangiosarcoma; lymphangioma; lymphangiomyoma; lymphangiosarcoma; pinealoma; carcinosarcoma; chondrosarcoma; cystosarcoma phyllodes; fibrosarcoma; hemangiosarcoma; leiomyosarcoma; leukosarcoma; liposarcoma; lymphangiosarcoma; myosarcoma; myxosarcoma; ovarian carcinoma; rhabdomyosarcoma; sarcoma; neoplasms; nerofibromatosis; and cervical dysplasia.

The therapeutic agents or pharmaceutical compositions that treat or reduce the risk of developing a hyperproliferative disease provided herein are administered to a subject who has or is at risk of developing a hyperproliferative disease at a therapeutically effective amount or dose. Such a dose may be determined or adjusted depending on various factors including the specific therapeutic agents or pharmaceutical compositions, the routes of administration, the subject's condition, that is, stage of the disease, severity of symptoms caused by the disease, general health status, as well as age, gender, and weight, and other factors apparent to a person skilled in the medical art. Similarly, the dose of the therapeutic for treating a hyperproliferative disease may be determined according to parameters understood by a person skilled in the medical art. When referring to a combination, a therapeutically effective dose refers to combined amounts of the active ingredients that result in the therapeutic effect, whether administered serially or simultaneously (in the same formulation or concurrently in separate formulations). Optimal doses may generally be determined using experimental models and/or clinical trials. Design and execution of pre-clinical and clinical studies for a therapeutic agent (including when administered for prophylactic benefit) described herein are well within the skill of a person skilled in the relevant art.

Generally, the therapeutic agent (e.g., MNK inhibitor) is administered at a therapeutically effective amount or dose. A therapeutically effective amount or dose will vary according to several factors, including the chosen route of administration, formulation of the composition, patient response, severity of the condition, the subject's weight, and the judgment of the prescribing physician. The dosage can be increased or decreased over time, as required by an individual patient. In certain instances, a patient initially is given a low dose, which is then increased to an efficacious dosage tolerable to the patient. Determination of an effective amount is well within the capability of those skilled in the art.

The route of administration of a therapeutic agent can be oral, intraperitoneal, transdermal, subcutaneous, by intravenous or intramuscular injection, by inhalation, topical, intralesional, infusion; liposome-mediated delivery; topical, intrathecal, gingival pocket, rectal, intrabronchial, nasal, transmucosal, intestinal, ocular or otic delivery, or any other methods known in the art.

In some embodiments, a therapeutic agent is formulated as a pharmaceutical composition. In some embodiments, a pharmaceutical composition incorporates particulate forms, protective coatings, protease inhibitors, or permeation enhancers for various routes of administration, including parenteral, pulmonary, nasal and oral. The pharmaceutical compositions can be administered in a variety of unit dosage forms depending upon the method/mode of administration. Suitable unit dosage forms, including powders, tablets, pills, capsules, lozenges, suppositories, patches, nasal sprays, injectables, implantable sustained-release formulations, etc.

In some embodiments, a pharmaceutical composition comprises an acceptable diluent, carrier or excipient. A pharmaceutically acceptable carrier includes any solvent, dispersion media, or coating that are physiologically compatible and that preferably do not interfere with or otherwise inhibit the activity of the therapeutic agent. Preferably, a carrier is suitable for intravenous, intramuscular, oral, intraperitoneal, transdermal, topical, or subcutaneous administration. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers. Other pharmaceutically acceptable carriers and their formulations are well-known and generally described in, for example, Remington: The Science and Practice of Pharmacy, 21st Edition, Philadelphia, Pa. Lippincott Williams & Wilkins, 2005. Various pharmaceutically acceptable excipients are well-known in the art and can be found in, for example, Handbook of Pharmaceutical Excipients (5^{th} ed., Ed. Rowe et al., Pharmaceutical Press, Washington, D.C.).

### EXAMPLES

### Example 1

### Effect of MNK mnk Inhibition on Hyperproliferative Disease

MNK inhibitors of this disclosure are potent and selective inhibitor of mitogen-activated protein kinase-interacting serine/threonine kinase-1 (MNK-1) and MNK-2.

Published studies have shown that dysregulated translation of messenger RNA (mRNA) plays a role in the pathogenesis of multiple solid tumors and hematological malignancies. MNK-1 and MNK-2 integrate signals from several pathways by phosphorylating eukaryotic initiation factor 4E and other proteins involved in mRNA translation. MNK inhibitors of this disclosure (e.g., Compound 107) potently blocks phosphorylation and activation of eIF4E, thereby selectively regulating translation of a small set of mRNA.

MNK kinases have been shown to integrate signals emanating from Toll-like receptors to regulate pro-inflammatory cytokines (Joshi et al., Biomol. Concepts 3:127, 2012; Rowlett et al., Am. J. Physiol. Gastointest. Liver Physiol. 294:G452-G459, 2007). Ribosome profiling was used to identify which genes are translationally and transcriptional modulated upon treatment with Compound 107 in TMD8 (diffuse large B cell lymphoma) cells, which harbor an activating mutation in MYD88 and exhibit constitutive TLR pathway signaling.

Translational profiling was used to identify the MNK regulon in the TMD8 DLBCL cell line. Concentration and time dependence of Compound 107 was evaluated to identify the translationally regulated MNK-sensitive gene set on a genome-wide scale.

### Procedures

(a) Cell Culture

TMD8 human diffuse large B-cell lymphoma line was cultured in RPMI media supplemented with penicillin G (100 U/ml), streptomycin (100 μg/ml), 10% FBS in a humidified atmosphere of 5% CO2 maintained at 37° C.
(b) Drug Treatment

TMD8 cells were seeded prior to drug treatment. The following day, cells were treated with either DMSO (vehicle control) or MNK inhibitor (Compound 107) at the appropriate dose and time.
(c) Immunoblotting

Cells were spun down and washed with PBS and lysed in 1× RIPA buffer (Thermo Fisher) for 15 min at 4° C. Lysates were sonicated briefly and clarified by centrifugation for 15 min at 14,000 rpm and supernatants were collected. Protein concentration in the soluble fraction was determined by BCA protein assay (Thermo Scientific). 20 μg of protein were resolved on 4-20% Bis-Tris gradient gel (Invitrogen) and transferred to nitrocellulose membrane. The resulting blots were blocked for 1 hr at room temperature with Odyssey blocking solution (LI-COR) and then incubated with anti-phospho-eIF4E (Millipore), anti-eIF4E (Santa Cruz), anti-CCND3 (Cell Signaling) or anti-IRF7 (Cell Signaling) at 4° C. overnight. I3-actin was used as a loading control. The following day, the blots were washed 3 times, 10 min each in TBST, and incubated with fluorescent conjugated secondary antibody for 1 hour at room temperature. The blots were then washed and scanned, specific proteins were detected by using the LI-COR Odyssey infrared imager.
(d) Non-Radioactive Nascent Protein Synthesis Assay

Newly synthesized proteins were detected by using the CLICK-IT Biotin Protein Analysis Detection Kit (Life Technologies; C33372) according to the manufacturer's protocol. Briefly, cells were rinsed with PBS once and incubated in methionine-free media for 30 min in the presence of the compound before being pulsed with the nonradioactive azide-containing methionine analogue AHA for 2 hrs. Cell lysates were then collected for the labeling reactions. The newly synthesized, AHA-incorporated protein was crosslinked to alkyne-derivatized biotin by a copper (I)—catalyzed cycloaddition (CLICK-IT) according to the manufacturer's instructions (Life Technologies). Following the labeling reactions, proteins were precipitated and quantified and subsequently subjected to immunoblotting analysis. Anti-streptavidin-HRP was used to detect newly synthesized proteins that contained biotin-conjugated AHA. Amount of newly synthesized proteins can be quantified by densitometry.
(e) Polysome Profiling

Cells were washed with cold PBS supplemented with cycloheximide and lysed with 1× mammalian cell lysis buffer for 10 minutes on ice. Lysates were clarified by centrifugation for 10 minutes at 14,000 rpm and supernatants were collected. The clarified lysate was loaded onto a 10-50% sucrose gradient containing 0.1 mg/ml cycloheximide (gradients were prepared using a BioComp Gradient Station) and centrifuged at 40,000 rpm for 2 hours at 4° C. using a SW40Ti rotor in a BECKMAN COULTER Optima L8-80M ultra centrifuge. Polysome fractions were isolated using the BioComp Gradient Station.
(f) Ribosome Profiling

Ribosomal profiling allows for measurement of changes in transcription and translation on a genome-wide basis accompanying inhibition of MNK with Compound 107 treatment of human DLBCL cells. Ribosome profiles of the Compound 107 treated TMD8 cells (about 3×10⁶ cells/10 cm plate were harvested for ribosome profiling following drug treatment) were prepared and analyzed for changes in translational efficiencies with respect to potential disease-associated cellular changes accompanying MNK inhibition.

Briefly, cells were washed with cold PBS supplemented with cycloheximide and lysed with 1× mammalian cell lysis buffer for 10 minutes on ice. Lysates were clarified by centrifugation for 10 minutes at 14,000 rpm and supernatants were collected. Cell lysates were processed to generate ribosomal protected fragments and total mRNA according to the instructions included with the ARTSEQ Ribosome Profiling Kit (Illumina). Sequencing of total RNA (RNA) and of ribosome-protected fragments of RNA (RPF) was carried out using RNA-Seq methodology according to the manufacturer's instructions (Illumina). To analyze the ribosomal profiles, RNA-Seq reads were processed with tools from the FASTX-Toolkit (fastq quality trimmer, fastx clipper and fastx trimmer). Unprocessed and processed reads were evaluated for a variety of quality measures using FastQC. Processed reads were mapped to the human genome using Tophat. Gene-by-gene assessment of the number of fragments strictly and uniquely mapping to the coding region of each gene was conducted using HTSeq-count, a component of the HTSeq package. Differential analyses of Compound 107 treatment of TMD8 cells were carried out with the software packages DESeq for transcription (RNA counts) and translational rate (RPF counts) and BABEL (Olshen 2013) for translational efficiency based upon ribosomal occupancy (RPF counts) as a function of RNA level (RNA counts). The Log₂ fold change in translational efficiency (TE) between drug treated and control is determined from the Log₂ fold change difference in RPF and RNA values (drug treated versus to control).

Genes with low counts in either RPF or RNA were excluded from differential analyses. Biological process classification was done using Gene Ontology term analysis.

### Results

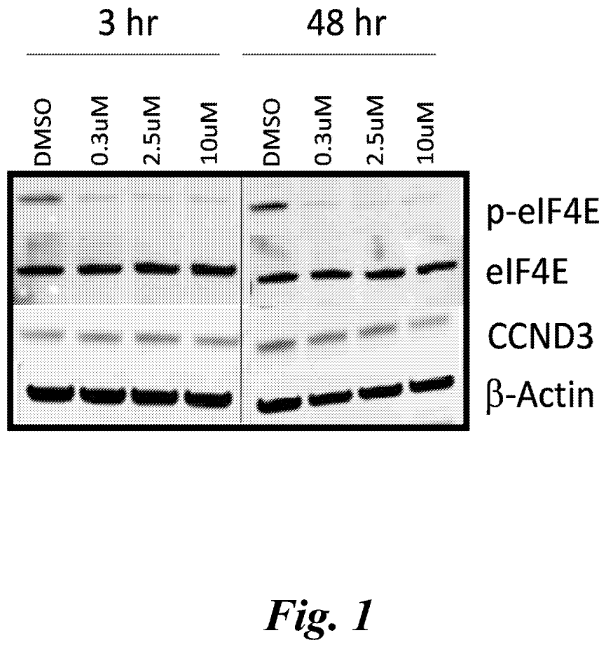

Compound 107 is a potent, highly selective MNK1 and MNK2 inhibitor. Treatment of TMD8 cells with Compound 107 (0.3-10 μM) for either 3 or 48 hours led to essentially complete inhibition of eIF4E phosphorylation at 5209 (FIG. 1). This is consistent with Compound 107 being a potent MNK inhibitor with a reported ECso value of 9.7 nM for inhibition of p-eIF4E in the TMD8 cell line (study report ECB-003). Exposure of TMD8 cells to increasing concentrations of Compound 107 caused a reduction in cyclin D3 at 48 hours (FIG. 1) and promoted cell survival (data not shown). Cyclin D3 is an important regulator of cell cycle (G1 to S phase) and a prognostic factor associated with poor clinical outcome in patients with DLBCL.

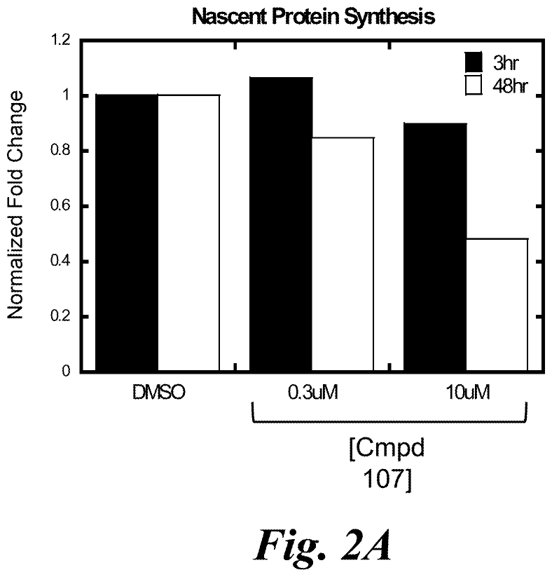

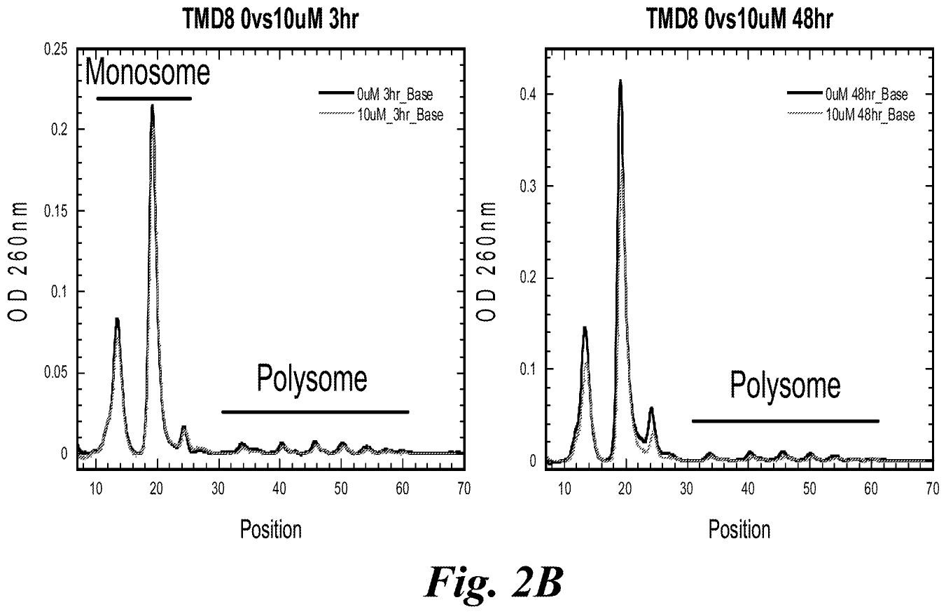

The effect of P-eIF4E inhibition on protein synthesis was measured by incorporation of non-radioactive methionine into newly synthesized proteins. Compound 107 treatment with either 0.3 or 10 μM for 3 hours and 0.3 μM for 48 hours had no impact on global protein synthesis whereas incubation of TMD8 cells with 10 μM Compound 107 for 48 hours showed a modest reduction in global protein synthesis rates (FIG. 2). It should be noted that the proliferation EC₅₀ for Compound 107 treatment of TMD8 cells is 3.3 μM; therefore, the decrease in nascent protein synthesis observed at longer incubation times may be due to a reduction in cellular proliferation.

To further evaluate the effect of inhibition of phosphorylation of eIF4E on translational regulation, we analyzed the polysome profiles of TMD8 cells in the presence or absence of treatment with 0.3 or 10 μM Compound 107 for 3 or 48 hours. Initiation inhibitors have been shown to shift the mRNA from actively translating polyribosomes to monosomes (Tscherne 1975); however, Compound 107 inhibition of eIF4E phosphorylation was not observed to alter the polysome to monosome distribution as a function of time or concentration of drug treatment (FIG. 2). This data is supportive of Compound 107 translationally regulating a small focused set of genes that do not result in substantial modulation of the polysome profile.

TMD8 cells were treated with DMSO or Compound 107 (0.3 or 10 μM) and cell lysates from two biological replicates were collected after 3 or 48 hours after treatment. The cell lysates were divided into two fractions and processed to quantitate the drug effects on the total mRNA (transcriptome) or RNase digested to generate the ribosome protected fragments (translatome). The raw sequencing counts were analyzed using DESeq analysis to determine differential expression or differential ribosome occupancy between DMSO and Compound 107 treatment and are reported as the loge fold change. Quantitation of the ribosome protected fragments (RPF) directly reflects the extent that a given transcript is bound by ribosomes and is a measure of the drug effects on translational rate.

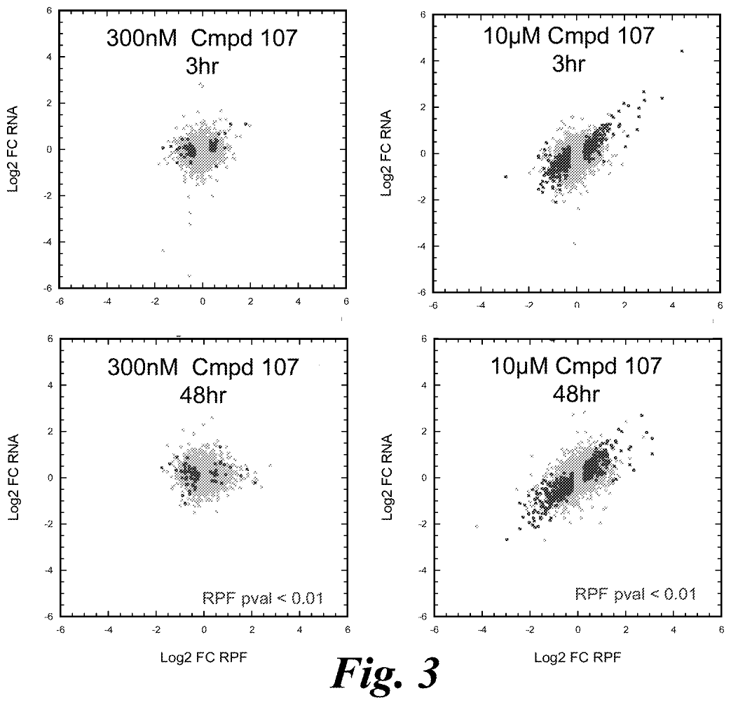

On a genome wide evaluation, inhibition of MNK1/2 resulted in the statistically significant modulation of translation rate or transcript levels for a small subset of genes after treatment of TMD8 cells with 300 nM or 10 μM Compound 107 for 3 or 48 hours (see FIG. 3, data points shown in blue have modulation in translational rate or ribosome protected fragments (RPF) that are statistically significant with a p-value <0.01).

The number of genes that were regulated at the translational rate (RPF) or translational efficiency (TE) is summarized in Table 2. Loge fold change in TE values are calculated from the difference in loge fold change in translational rate (RPF) between drug treated and control, and the loge fold change in total mRNA (drug treated vs. control). Evaluation of TE results in normalizing translation changes to transcript abundance. The statistical significance for TE values was determined using Babel software which was developed for assessing the significance of changes in translational regulation between conditions.

TABLE 2
Summary of Gene Modulation from Ribosome Profiling

| Cmpd | Time Point | Mode of | # Genes | # Genes |
|---|---|---|---|---|
| 107 | (hour) | Regulation | Down Regulated | Up Regulated |
| 10 μM | 3 | Translational | 58 | 64 |
| | | Rate (RPF) | | |
| 10 μM | 3 | Translational | 24 | 27 |
| | | Efficiency | | |
| 10 μM | 48 | Translational | 123 | 92 |
| | | Rate (RPF) | | |
| 10 μM | 48 | Translational | 31 | 15 |
| | | Efficiency | | |

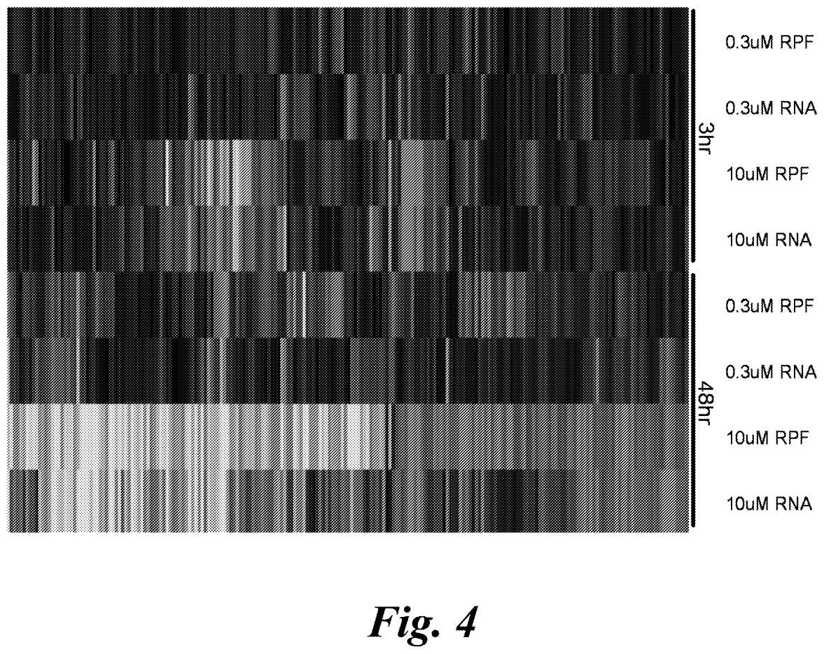

Ribosome profiling identified 123 genes with decreased translational rate after treatment of TMD8 cells with 1004 Compound 107 for 48 hours relative to DMSO control (log₂≤−0.75, p-value <0.01). Of these 123 MNK-sensitive genes, 51 were also down regulated at the mRNA expression level (log₂≤−0.75, p-value <0.01); whereas 27 were selectively down regulated at the translational level in the absence of substantial transcript changes (log₂≤−0.75, p-value <0.05). In addition, 92 genes were identified with increased translational rate with MNK inhibition (log₂≥0.75, p-value <0.01), see Table 3. Even though all of these genes were not identified as statistically significant at a lower concentration or the 3 hour time point of Compound 107 treatment, the heatmap shown in FIG. 4 illustrates that dose dependent or time dependent modulation was observed for many of the genes (see gene lists in Table 3 and Table 4).

Characterization of MNK Regulon

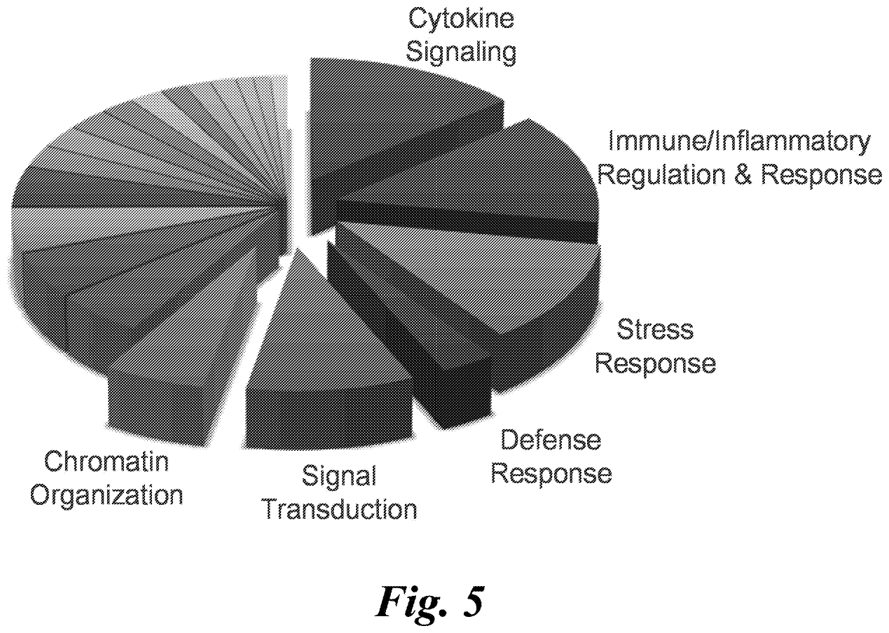

The ribosome profiling data was analyzed to help elucidate what role MNK plays in regulating biological function. As shown in FIG. 5, MNK-sensitive genes cluster into functional categories known to be involved in the development and progression of cancer.

A significant portion of MNK-sensitive genes cluster in the cytokine mediated signaling, immune/inflammatory regulation and response, and stress response functional categories indicaqting that these genes define an important MNK regulon.

One of the major functional classifications, regulation of immune and inflammatory response, identified lymphocyte-activation gene 3 (LAG-3). LAG-3 plays an important role in tumor mediated immune suppression. Antibody treatment to block LAG-3 in cancer demonstrated enhanced activation of T cells at the tumor site leading to disruption of tumor growth (Mahoney 2015). LAG-3 was translationally regulated by MNK inhibition with a decrease in ribosome occupancy observed in the absence of a substantial change in mRNA expression levels (Table 3). Treating TMD8 cells with Compound 107 resulted in a selective decrease in the expression of LAG-3 at the protein level (data not shown). A second immune checkpoint inhibitor, programmed cell death 1 (PD-1), was also observed to be translationally down regulated (˜2-fold) by Compound 107 treatment. PD-1 regulation did not meet the statistical significance cutoff (p-value=0.033); therefore, the modulation is not captured in Table 3. Further analysis confirmed that levels of PD-1 on the surface of activated T cells were reduced upon incubation with Compound 107 (data not shown).

TABLE 3
Gene Signature with altered Translational Efficiency -
48 hr treatment with Compound 107 (10 μM)

| | | Log2 FC | |
|---|---|---|---|
| | | Translational | Babel |
| ENSEMBL | HGNC | Efficiency | p-value |
| ENSG00000162545 | CAMK2N1 | −2.5546 | 1.15E−07 |
| ENSG00000177595 | PIDD | −1.62939 | 2.03E−07 |
| ENSG00000124444 | ZNF576 | −1.51934 | 0.000764 |
| ENSG00000126458 | RRAS | −1.4383 | 0.00069 |
| ENSG00000241370 | RPP21 | −1.42971 | 0.000907 |
| ENSG00000165795 | NDRG2 | −1.39889 | 5.41E−05 |
| ENSG00000130193 | C8orf55 | −1.37918 | 6.53E−07 |
| ENSG00000119862 | LGALSL | −1.37257 | 0.000486 |
| ENSG00000172830 | SSH3 | −1.3681 | 1.88E−06 |
| ENSG00000163704 | PRRT3 | −1.35517 | 0.001371 |
| ENSG00000174165 | ZDHHC24 | −1.35091 | 1.70E−05 |
| ENSG00000185818 | NAT8L | −1.33508 | 1.32E−05 |
| ENSG00000198959 | TGM2 | −1.31951 | 2.08E−06 |
| ENSG00000135127 | CCDC64 | −1.31264 | 0.00323 |
| ENSG00000123146 | CD97 | −1.31209 | 1.38E−05 |
| ENSG00000182319 | PRAGMIN.1 | −1.25028 | 3.51E−07 |
| ENSG00000126259 | KIRREL2 | −1.24828 | 0.000444 |
| ENSG00000135736 | CCDC102A | −1.23619 | 0.000324 |
| ENSG00000162419 | GMEB1 | −1.22516 | 0.000687 |
| ENSG00000088836 | SLC4A11 | −1.21669 | 0.006112 |
| ENSG00000173264 | GPR137 | −1.20785 | 5.72E−05 |
| ENSG00000161677 | JOSD2 | −1.18989 | 4.09E−05 |
| ENSG00000259207 | ITGB3 | −1.17172 | 0.009106 |
| ENSG00000172375 | C2CD2L | −1.16622 | 0.000622 |
| ENSG00000166188 | ZNF319 | −1.10642 | 0.000302 |
| ENSG00000089692 | LAG3 | −1.08355 | 0.00325 |
| ENSG00000224877 | C17orf89 | −1.03812 | 0.000167 |
| ENSG00000080493 | SLC4A4 | −1.03449 | 0.009316 |
| ENSG00000006118 | TMEM132A | −1.01053 | 0.00187 |
| ENSG00000166145 | SPINT1 | −1.00409 | 0.000647 |
| ENSG00000166165 | CKB | −1.00108 | 0.002018 |
| ENSG00000196659 | TTC30B | 1.019655 | 0.002671 |
| ENSG00000163703 | CRELD1 | 1.083448 | 0.00687 |
| ENSG00000133111 | RFXAP | 1.085844 | 0.002703 |
| ENSG00000135919 | SERPINE2 | 1.099489 | 9.75E−05 |
| ENSG00000131669 | NINJ1 | 1.155379 | 0.000377 |
| ENSG00000204271 | SPIN3 | 1.373141 | 0.000295 |
| ENSG00000145730 | PAM | 1.482688 | 0.00044 |
| ENSG00000103066 | PLA2G15 | 1.514339 | 5.86E−05 |
| ENSG00000093072 | CECR1 | 1.544801 | 1.90E−08 |
| ENSG00000085741 | WNT11 | 1.586229 | 3.94E−06 |
| ENSG00000174943 | KCTD13 | 1.59024 | 7.09E−06 |
| ENSG00000197121 | PGAP1 | 1.597591 | 2.90E−05 |
| ENSG00000136197 | C7orf25 | 1.672952 | 0.002464 |
| ENSG00000008441 | NFIX | 1.854321 | 6.30E−09 |
| ENSG00000154229 | PRKCA | 1.88062 | 7.53E−10 |

A decrease in translation rate with Compound 107 treatment was also observed for a number of immune/inflammatory regulation and responsive genes (e.g., TNF, IL6, IL10, IL12B, STAT5A and CD97), many of which have been reported to be frequently upregulated in cancer. Evaluation of select cytokine/chemokine biomarkers (CXCL10, IL6 and IL10) confirmed that they were also down regulated at the protein expression level with drug treatment (data not shown).

CD97 antigen was also identified to be translationally down regulated with MNK inhibition (see Table 3). Drug treatment caused a substantial reduction in ribosome occupancy with minimal changes in total mRNA suggesting that regulation is predominately by translation inhibition. CD97 plays a role in mediating immune defense, inflammation as well as cell adhesion and migration (Safaee et al., Intern. J. Oncol. 43:1343, 2013). Interaction between CD97 and its ligand CD55 regulates proliferation and INFγ secretion. This receptor also plays a role in leukocyte migration and has been reported to be overexpressed in many cancer types. The expression levels have been reported to correlate with migration and invasion in tumor cell lines (Liu et al., PLoS ONE 7:e39989, 2012).

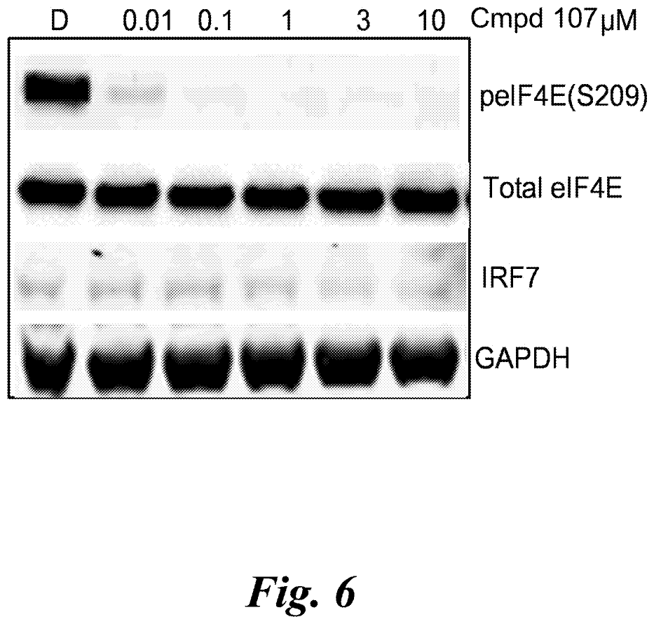

The cytokine mediated signaling functional classification was also found to contain a regulator of interferon responsive gene (IRF7) that was translationally regulated in the absence of mRNA level changes by MNK inhibition. It has been reported that the translation of the transcription factor IRF7, a master regulator of interferon sensitive genes, was sensitive to changes in levels of the eIF4F complex (Colina et al., Nature 452:323, 2008). Increased concentrations of Compound 107 caused a decrease of eIF4G bound to eIF4E. This reduces the levels of eIF4F complex resulting in decreased translation of IRF7 and downregulates the production of interferon sensitive genes in TMD8 cells. Interestingly, many interferon responsive genes (e.g., IFITM1, IFITM2, IFIT5, IFI6, IFI27, IFI44L, OAS1, OAS2, OAS3 and OASL) were observed to be modulated at their translational rate by MNK inhibition suggesting that IRF7 may play a role in regulating the expression level of these genes. Treatment of TMD8 cells with Compound 107 confirmed that IRF7 was decreased at the protein level (FIG. 6).

TABLE 4
Gene Signature with altered Translational Rate -
48 hr treatment with Compound 107 (10 μM)

| | | Log2 FC | |
|---|---|---|---|
| | | RPF(508)/ | RPF |
| ENSEMBL | HGNC | RPF(DMSO) | p-value |
| ENSG00000113302 | IL12B | −2.97926 | 7.17E−08 |
| ENSG00000136634 | IL10 | −2.44428 | 1.01E−22 |
| ENSG00000126561 | STAT5A | −2.41633 | 0.003011 |
| ENSG00000099377 | HSD3B7 | −2.28789 | 0.000157 |
| ENSG00000188095 | MESP2 | −2.24576 | 0.002221 |
| ENSG00000123146 | CD97 | −2.07735 | 0.000431 |
| ENSG00000115155 | OTOF | −2.06598 | 1.37E−12 |
| ENSG00000163435 | ELF3 | −2.05078 | 0.003752 |
| ENSG00000162896 | PIGR | −2.04028 | 4.33E−21 |
| ENSG00000038427 | VCAN | −2.01837 | 0.000273 |
| ENSG00000100385 | IL2RB | −1.91206 | 0.000143 |
| ENSG00000165795 | NDRG2 | −1.90564 | 0.00301 |
| ENSG00000185745 | IFIT1 | −1.85909 | 3.90E−07 |
| ENSG00000169245 | CXCL10 | −1.85291 | 3.33E−05 |
| ENSG00000061492 | WNT8A | −1.81845 | 4.63E−05 |
| ENSG00000050730 | TNIP3 | −1.80473 | 2.65E−07 |
| ENSG00000232810 | TNF | −1.79413 | 0.002099 |
| ENSG00000102962 | CCL22 | −1.78462 | 2.35E−13 |
| ENSG00000131650 | KREMEN2 | −1.76152 | 0.005495 |
| ENSG00000089692 | LAG3 | −1.73946 | 1.93E−08 |
| ENSG00000089327 | FXYD5 | −1.72417 | 2.24E−05 |
| ENSG00000159403 | C1R | −1.71429 | 0.004739 |
| ENSG00000166165 | CKB | −1.69242 | 0.000332 |
| ENSG00000196154 | S100A4 | −1.66603 | 3.59E−12 |
| ENSG00000197471 | SPN | −1.66209 | 1.92E−06 |
| ENSG00000104974 | LILRA1 | −1.61062 | 0.000876 |
| ENSG00000119917 | IFIT3 | −1.58089 | 1.28E−08 |
| ENSG00000117228 | GBP1 | −1.58032 | 0.000907 |
| ENSG00000100300 | TSPO | −1.57417 | 1.15E−05 |
| ENSG00000143554 | SLC27A3 | −1.51692 | 6.60E−05 |
| ENSG00000165949 | IFI27 | −1.49986 | 1.16E−05 |
| ENSG00000130589 | RP4-697K14.7.1 | −1.48475 | 3.67E−05 |
| ENSG00000080493 | SLC4A4 | −1.48412 | 0.004421 |
| ENSG00000126259 | KIRREL2 | −1.48401 | 0.004371 |
| ENSG00000196433 | ASMT | −1.45577 | 1.08E−32 |
| ENSG00000133106 | EPSTI1 | −1.43374 | 1.86E−09 |
| ENSG00000198959 | TGM2 | −1.42209 | 0.002437 |
| ENSG00000155367 | PPM1J | −1.40931 | 0.005481 |
| ENSG00000197594 | ENPP1 | −1.37762 | 0.001932 |
| ENSG00000162419 | GMEB1 | −1.36949 | 0.006765 |
| ENSG00000111331 | OAS3 | −1.36883 | 1.65E−14 |
| ENSG00000198734 | F5 | −1.36313 | 0.000451 |
| ENSG00000184979 | USP18 | −1.35151 | 0.008731 |
| ENSG00000137628 | DDX60 | −1.33852 | 2.25E−07 |
| ENSG00000143545 | RAB13 | −1.33443 | 0.007806 |
| ENSG00000204580 | DDR1 | −1.32544 | 0.002022 |
| ENSG00000073737 | DHRS9 | −1.32189 | 7.03E−25 |
| ENSG00000135245 | HILPDA | −1.32053 | 0.001451 |
| ENSG00000132530 | XAF1 | −1.31584 | 0.001575 |
| ENSG00000109654 | TRIM2 | −1.30871 | 0.008596 |
| ENSG00000166145 | SPINT1 | −1.27736 | 0.001682 |
| ENSG00000197409 | HIST1H3D | −1.26779 | 2.71E−18 |
| ENSG00000166016 | ABTB2 | −1.26672 | 7.00E−10 |
| ENSG00000198339 | HIST1H4I | −1.26529 | 4.22E−15 |
| ENSG00000166825 | ANPEP | −1.26362 | 2.94E−18 |
| ENSG00000076706 | MCAM | −1.24795 | 0.003741 |
| ENSG00000197956 | S100A6 | −1.22498 | 3.82E−12 |
| ENSG00000148671 | C10orf116 | −1.22396 | 0.000479 |
| ENSG00000146094 | DOK3 | −1.20264 | 1.84E−08 |
| ENSG00000197355 | UAP1L1 | −1.2022 | 0.003814 |
| ENSG00000119698 | PPP4R4 | −1.19834 | 0.004533 |
| ENSG00000152778 | IFIT5 | −1.18044 | 7.33E−08 |
| ENSG00000100097 | LGALS1 | −1.17449 | 5.59E−27 |
| ENSG00000129226 | CD68 | −1.17278 | 6.84E−06 |
| ENSG00000135114 | OASL | −1.16832 | 0.006662 |
| ENSG00000188987 | HIST1H4D | −1.16014 | 3.38E−10 |
| ENSG00000143851 | PTPN7 | −1.16005 | 0.000213 |
| ENSG00000196226 | HIST1H2BB | −1.14935 | 1.69E−25 |
| ENSG00000135363 | LMO2 | −1.14579 | 3.62E−10 |
| ENSG00000137959 | IFI44L | −1.12524 | 0.007482 |
| ENSG00000118785 | SPP1 | −1.11429 | 0.005206 |
| ENSG00000170054 | SERPINA9 | −1.10919 | 0.007474 |
| ENSG00000086730 | LAT2 | −1.10583 | 1.84E−05 |
| ENSG00000121858 | TNFSF10 | −1.0941 | 0.005782 |
| ENSG00000182319 | PRAGMIN.1 | −1.08519 | 0.000324 |
| ENSG00000033327 | GAB2 | −1.06871 | 1.40E−07 |
| ENSG00000111335 | OAS2 | −1.05905 | 3.64E−12 |
| ENSG00000105246 | EBI3 | −1.05549 | 8.49E−10 |
| ENSG00000106211 | HSPB1 | −1.05329 | 0.000258 |
| ENSG00000198374 | HIST1H2AL | −1.02619 | 3.56E−18 |
| ENSG00000197747 | S100A10 | −1.02099 | 0.004346 |
| ENSG00000172578 | KLHL6 | −1.01847 | 3.63E−11 |
| ENSG00000175793 | SFN | −1.01447 | 1.08E−07 |
| ENSG00000126709 | IFI6 | −1.00881 | 0.000183 |
| ENSG00000185885 | IFITM1 | −0.99804 | 5.83E−05 |
| ENSG00000157601 | MX1 | −0.9881 | 8.09E−18 |
| ENSG00000078900 | TP73 | −0.98363 | 0.001645 |
| ENSG00000050344 | NFE2L3 | −0.97099 | 0.005737 |
| ENSG00000187608 | ISG15 | −0.96993 | 2.60E−05 |
| ENSG00000244509 | APOBEC3C | −0.95392 | 7.73E−07 |
| ENSG00000105339 | DENND3 | −0.9497 | 6.50E−10 |
| ENSG00000185201 | IFITM2 | −0.94598 | 0.00069 |
| ENSG00000116701 | NCF2 | −0.93045 | 2.78E−10 |
| ENSG00000203813 | HIST1H3H | −0.92611 | 3.60E−16 |
| ENSG00000127838 | PNKD | −0.92541 | 0.000878 |
| ENSG00000136048 | DRAM1 | −0.9214 | 0.008978 |
| ENSG00000011600 | TYROBP | −0.92083 | 3.35E−05 |
| ENSG00000069424 | KCNAB2 | −0.91626 | 0.000571 |
| ENSG00000168961 | LGALS9 | −0.91586 | 0.002821 |
| ENSG00000142227 | EMP3 | −0.91273 | 5.13E−12 |
| ENSG00000256018 | HIST1H3G | −0.90594 | 1.58E−23 |
| ENSG00000112297 | AIM1 | −0.90393 | 0.009322 |
| ENSG00000137880 | GCHFR | −0.90347 | 0.005474 |
| ENSG00000160193 | WDR4 | −0.90196 | 0.002151 |
| ENSG00000185507 | IRF7 | −0.8927 | 3.68E−05 |
| ENSG00000138642 | HERC6 | −0.8905 | 6.66E−09 |
| ENSG00000075643 | MOCOS | −0.88696 | 6.12E−05 |
| ENSG00000120756 | PLS1 | −0.85114 | 0.008274 |
| ENSG00000256872 | NOL12.1 | −0.84317 | 0.002179 |
| ENSG00000075673 | ATP12A | −0.84142 | 0.004515 |
| ENSG00000135678 | CPM | −0.82877 | 7.15E−05 |
| ENSG00000196374 | HIST1H2BM | −0.82148 | 2.76E−16 |
| ENSG00000105501 | SIGLEC5 | −0.81396 | 1.27E−05 |
| ENSG00000069956 | MAPK6 | −0.81322 | 0.005761 |
| ENSG00000168298 | HIST1H1E | −0.80114 | 3.94E−08 |
| ENSG00000184348 | HIST1H2AK | −0.7894 | 0.000417 |
| ENSG00000100628 | ASB2 | −0.78613 | 0.000237 |
| ENSG00000204388 | HSPA1B | −0.77891 | 0.002023 |
| ENSG00000182150 | C9orf102 | −0.77522 | 1.27E−05 |
| ENSG00000161642 | ZNF385A | −0.77484 | 0.0067 |
| ENSG00000111679 | PTPN6 | −0.76789 | 1.18E−17 |
| ENSG00000089127 | OAS1 | −0.76434 | 4.93E−08 |
| ENSG00000115415 | STAT1 | −0.75629 | 1.73E−11 |
| ENSG00000119801 | YPEL5 | 0.760448 | 0.000299 |
| ENSG00000181192 | DHTKD1 | 0.764402 | 8.67E−08 |
| ENSG00000136816 | TOR1B | 0.768829 | 0.009313 |
| ENSG00000047346 | FAM214A | 0.777436 | 0.003669 |
| ENSG00000142197 | DOPEY2 | 0.777617 | 0.004336 |
| ENSG00000160190 | SLC37A1 | 0.778985 | 0.002634 |
| ENSG00000147813 | NAPRT1 | 0.779163 | 0.002601 |
| ENSG00000182197 | EXT1 | 0.781067 | 0.003696 |
| ENSG00000137522 | RNF121 | 0.782531 | 0.000106 |
| ENSG00000085733 | CTTN | 0.79859 | 0.000857 |
| ENSG00000198799 | LRIG2 | 0.811407 | 0.002378 |
| ENSG00000040933 | INPP4A | 0.81253 | 0.00019 |
| ENSG00000117115 | PADI2 | 0.827014 | 4.16E−07 |
| ENSG00000237541 | HLA-DQA2 | 0.83794 | 0.00026 |
| ENSG00000064687 | ABCA7 | 0.838349 | 8.92E−08 |
| ENSG00000178567 | EPM2AIP1 | 0.844625 | 1.10E−07 |
| ENSG00000100239 | PPP6R2 | 0.868881 | 0.002086 |
| ENSG00000022567 | SLC45A4 | 0.882449 | 0.000642 |
| ENSG00000070190 | DAPP1 | 0.888139 | 1.44E−06 |
| ENSG00000011638 | TMEM159 | 0.892833 | 0.002825 |
| ENSG00000134508 | CABLES1 | 0.896359 | 3.19E−06 |
| ENSG00000068650 | ATP11A | 0.900461 | 1.41E−08 |
| ENSG00000162772 | ATF3 | 0.903429 | 6.89E−07 |
| ENSG00000184588 | PDE4B | 0.907944 | 4.92E−07 |
| ENSG00000104381 | GDAP1 | 0.910922 | 0.001954 |
| ENSG00000213983 | AP1G2 | 0.920318 | 4.05E−10 |
| ENSG00000007255 | TRAPPC6A | 0.921604 | 5.69E−06 |
| ENSG00000125089 | SH3TC1 | 0.92921 | 4.04E−09 |
| ENSG00000162849 | KIF26B | 0.937105 | 2.55E−08 |
| ENSG00000132465 | IGJ | 0.939334 | 0.001111 |
| ENSG00000112799 | LY86 | 0.939406 | 0.007118 |
| ENSG00000109323 | MANBA | 0.948906 | 8.51E−05 |
| ENSG00000177082 | WDR73 | 0.964199 | 0.001708 |
| ENSG00000120915 | EPHX2 | 0.965725 | 0.007941 |
| ENSG00000177169 | ULK1 | 0.971771 | 0.005245 |
| ENSG00000122547 | EEPD1 | 0.972615 | 0.002936 |
| ENSG00000155008 | APOOL | 0.994015 | 0.002942 |
| ENSG00000180879 | SSR4 | 1.02052 | 1.70E−08 |
| ENSG00000148450 | MSRB2 | 1.023757 | 0.004321 |
| ENSG00000168952 | STXBP6 | 1.025274 | 0.004935 |
| ENSG00000175866 | BAIAP2 | 1.033872 | 0.00088 |
| ENSG00000011523 | CEP68 | 1.047207 | 1.86E−06 |
| ENSG00000181104 | F2R | 1.050301 | 3.94E−07 |
| ENSG00000039068 | CDH1 | 1.063384 | 2.48E−28 |
| ENSG00000162804 | SNED1 | 1.072969 | 3.61E−06 |
| ENSG00000204186 | ZDBF2 | 1.074851 | 0.000103 |
| ENSG00000116741 | RGS2 | 1.081702 | 8.32E−12 |
| ENSG00000086062 | B4GALT1 | 1.083754 | 1.85E−24 |
| ENSG00000108469 | RECQL5 | 1.114083 | 0.003726 |
| ENSG00000149289 | ZC3H12C | 1.118735 | 0.002052 |
| ENSG00000063587 | ZNF275 | 1.124034 | 0.007157 |
| ENSG00000115902 | SLC1A4 | 1.124858 | 1.23E−19 |
| ENSG00000126353 | CCR7 | 1.125736 | 8.99E−14 |
| ENSG00000196735 | HLA-DQA1 | 1.13898 | 0.000388 |
| ENSG00000173511 | VEGFB | 1.141671 | 0.00834 |
| ENSG00000100422 | CERK | 1.153379 | 0.008695 |
| ENSG00000196586 | MYO6 | 1.15805 | 0.004386 |
| ENSG00000111863 | C6orf105 | 1.174363 | 0.004815 |
| ENSG00000102181 | CD99L2 | 1.191549 | 0.00115 |
| ENSG00000157613 | CREB3L1 | 1.1936 | 2.67E−10 |
| ENSG00000148814 | LRRC27 | 1.203632 | 0.001584 |
| ENSG00000175274 | TP53I11 | 1.207781 | 3.73E−12 |
| ENSG00000119139 | TJP2 | 1.221339 | 0.00027 |
| ENSG00000141655 | TNFRSF11A | 1.222958 | 9.83E−05 |
| ENSG00000091490 | SEL1L3 | 1.224037 | 4.42E−06 |
| ENSG00000146072 | TNFRSF21 | 1.237311 | 0.001914 |
| ENSG00000178498 | DTX3 | 1.257036 | 0.002195 |
| ENSG00000256043 | CTSO | 1.272534 | 0.007318 |
| ENSG00000054219 | LY75 | 1.302381 | 0.000595 |
| ENSG00000005379 | BZRAP1 | 1.306583 | 0.007345 |
| ENSG00000142494 | SLC47A1 | 1.413722 | 0.000421 |
| ENSG00000170873 | MTSS1 | 1.421876 | 4.08E−13 |
| ENSG00000137642 | SORL1 | 1.424223 | 2.81E−09 |
| ENSG00000197121 | PGAP1 | 1.475874 | 0.002179 |
| ENSG00000138152 | BTBD16 | 1.537078 | 0.003756 |
| ENSG00000101190 | TCFL5 | 1.562859 | 0.008093 |
| ENSG00000103316 | CRYM | 1.57238 | 2.36E−05 |
| ENSG00000136197 | C7orf25 | 1.591281 | 0.004133 |
| ENSG00000132872 | SYT4 | 1.599338 | 2.13E−06 |
| ENSG00000090104 | RGS1 | 1.612188 | 1.13E−07 |
| ENSG00000132622 | HSPA12B | 1.705048 | 0.000299 |
| ENSG00000144218 | AFF3 | 1.807852 | 3.82E−08 |
| ENSG00000170801 | HTRA3 | 1.822354 | 2.29E−09 |
| ENSG00000259207 | ITGB3 | 1.918161 | 0.00147 |
| ENSG00000168679 | SLC16A4 | 2.119988 | 0.000332 |
| ENSG00000176058 | TPRN | 2.164119 | 0.007139 |
| ENSG00000163545 | NUAK2 | 2.214125 | 0.003588 |
| ENSG00000144115 | THNSL2 | 2.340677 | 0.000963 |
| ENSG00000183508 | FAM46C | 2.666134 | 0.000144 |
| ENSG00000160307 | S100B | 2.854634 | 1.74E−07 |
| ENSG00000173890 | GPR160 | 3.098965 | 0.000295 |
| ENSG00000123096 | SSPN | 3.116769 | 0.001045 |

### Ribosome Profiling of Compound 107—Modulation of Translational Efficiency

The ribosome profiling data was also analyzed to identify genes that were modulated in translational efficiency (TE) by MNK inhibition (ribosome occupancy changes in the absence of modulation of total mRNA). Tables 2, 3 and 5 summarize the genes with altered translational efficiencies when TMD8 cells were treated with 10 μM Compound 107 for 3 or 48 hours (log₂≥|1.0|, p-value<0.01). Under all conditions tested, only a small subset of genes exhibited modulation in translational efficiency suggesting that MNK inhibition regulates the translation of a select set of genes. The short incubation time (3 hour) dataset was further evaluated in order to separate translational regulation from potential secondary effects of drug treatment. Within this treatment duration,

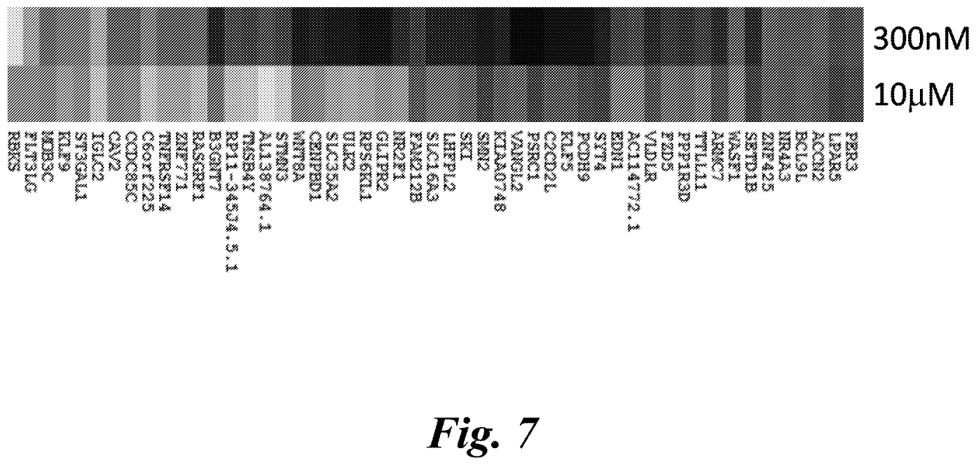

Compound 107 had negligible effects on global protein synthesis (see FIG. 2). Table 5 lists the genes identified to be translationally regulated with treatment of TMD8 cells with 10 μM Compound 107 for 3 hours. Comparison of the translational efficiencies for these genes after treatment with 300 nM or 10 μM Compound 107 shows a dose dependent regulation where 300 nM Compound 107 modulated the translational efficiency to an equal or lesser extent than at higher concentrations for the majority of genes identified (see FIG. 7).

TABLE 5
Gene Signature with Altered Translational Efficiency -
3 hr treatment with Compound 107 (10 μM)

| | | Log2 FC | |
|---|---|---|---|
| | | Translational | Babel |
| ENSEMBL | HGNC | Efficiency | p-value |
| ENSG00000226209 | AL138764.1 | −2.03046 | 1.54E−09 |
| ENSG00000197457 | STMN3 | −1.81242 | 1.36E−06 |
| ENSG00000203778 | C6orf225 | −1.75288 | 1.20E−07 |
| ENSG00000211677 | IGLC2 | −1.72128 | 2.06E−07 |
| ENSG00000154620 | TMSB4Y | −1.71261 | 0.00039 |
| ENSG00000261740 | RP11-345J4.5.1 | −1.69665 | 1.25E−07 |
| ENSG00000058335 | RASGRF1 | −1.66349 | 4.16E−07 |
| ENSG00000083290 | ULK2 | −1.49532 | 0.001886 |
| ENSG00000179965 | ZNF771 | −1.48497 | 2.68E−05 |
| ENSG00000157873 | TNFRSF14 | −1.44732 | 0.000508 |
| ENSG00000198208 | RPS6KL1 | −1.42512 | 0.00041 |
| ENSG00000102100 | SLC35A2 | −1.42482 | 0.000109 |
| ENSG00000175745 | NR2F1 | −1.41529 | 0.000296 |
| ENSG00000119138 | KLF9 | −1.38105 | 0.000254 |
| ENSG00000122694 | GLIPR2 | −1.32437 | 0.000314 |
| ENSG00000177946 | CENPBD1 | −1.25052 | 0.00118 |
| ENSG00000090554 | FLT3LG | −1.24394 | 0.000177 |
| ENSG00000171174 | RBKS | −1.22956 | 0.00027 |
| ENSG00000205476 | CCDC85C | −1.22506 | 0.000486 |
| ENSG00000008513 | ST3GAL1 | −1.21844 | 0.00017 |
| ENSG00000105971 | CAV2 | −1.16862 | 0.002859 |
| ENSG00000142961 | MOB3C | −1.16047 | 0.003302 |
| ENSG00000061492 | WNT8A | −1.15972 | 0.004508 |
| ENSG00000156966 | B3GNT7 | −1.02861 | 0.001106 |
| ENSG00000139718 | SETD1B | 1.007829 | 0.000121 |
| ENSG00000205571 | SMN2 | 1.008299 | 0.005058 |
| ENSG00000184574 | LPAR5 | 1.017622 | 0.003565 |
| ENSG00000132872 | SYT4 | 1.019057 | 0.001408 |
| ENSG00000134222 | PSRC1 | 1.03198 | 0.001867 |
| ENSG00000184226 | PCDH9 | 1.109301 | 0.007408 |
| ENSG00000049246 | PER3 | 1.112369 | 0.001577 |
| ENSG00000125449 | ARMC7 | 1.191173 | 0.001636 |
| ENSG00000147852 | VLDLR | 1.276713 | 2.67E−05 |
| ENSG00000197852 | FAM212B | 1.286305 | 0.004916 |
| ENSG00000110881 | ACCN2 | 1.307382 | 6.47E−05 |
| ENSG00000119508 | NR4A3 | 1.340635 | 8.58E−08 |
| ENSG00000102554 | KLF5 | 1.355587 | 0.002627 |
| ENSG00000135426 | KIAA0748 | 1.381165 | 4.52E−05 |
| ENSG00000172375 | C2CD2L | 1.391317 | 2.26E−06 |
| ENSG00000157933 | SKI | 1.466 | 9.39E−09 |
| ENSG00000186174 | BCL9L | 1.477711 | 0.000604 |
| ENSG00000204947 | ZNF425 | 1.506457 | 0.000563 |
| ENSG00000162738 | VANGL2 | 1.51594 | 1.44E−05 |
| ENSG00000132825 | PPP1R3D | 1.545654 | 6.53E−06 |
| ENSG00000145685 | LHFPL2 | 1.662132 | 1.84E−05 |
| ENSG00000141526 | SLC16A3 | 1.720928 | 9.66E−06 |
| ENSG00000163251 | FZD5 | 1.746008 | 1.91E−07 |
| ENSG00000175764 | TTLL11 | 1.895963 | 3.86E−11 |
| ENSG00000112290 | WASF1 | 2.072213 | 8.31E−11 |
| ENSG00000078401 | EDN1 | 2.357118 | 3.03E−08 |
| ENSG00000188971 | AC114772.1 | 2.425778 | 2.71E−06 |

Interestingly, a number of the translationally regulated MNK-sensitive genes have been reported to play a role in cancer development. Select highlighted genes were found to fall within four functional categories. (1) Post-Translational Modification: ST3GAL1 (ST3 beta-galactoside alpha-2,3-sialyltransferase 1) is involved in protein glycosylation—one of the most important posttranslational modifications of proteins. Increased sialytransferase activity promotes cancer cell metastasis and correlates with poor prognosis (Chen et al., Cancer Res. 71:473, 2011). Programmed cell death-1 (PD-1) is an immunoinhibitory receptor that plays a major role in tumor immune escape. PD-1 interacts with its ligand PD-Ll to inhibit T lymphocyte proliferation and survival (Mahoney et al., Nat. Rev. 14:561, 2015). The affinity of the PD-1/PD-L1 interaction is regulated by glycosylation. The non-glycosylated form of the proteins reduces the affinity by ˜35 fold suggesting that this MNK-sensitive gene may regulate tumor immune escape (Carlsson et al., J. Immunol. Clin. Res. 2:1013, 2014). In addition, ST3GAL1 has also been reported to be upregulated in breast cancer where aberrant glycosylation has been well documented (Sproviero et al., J. Biol. Chem. 287:44490, 2012). SLC35A2 (solute carrier family 35 (UDP-galactose transporter), member A2) transports the activated sugar, UDP-galactose, into Golgi vesicles where it transports the sugar for glycosylation and may position the glycosyltransferases for substrate binding (Sosicka et al., Biochem. Biophys. Res. Comm. 454:486, 2014). Increased expression of SLC35A2 has been reported in cancer (Kumamoto et al., Cancer Res. 61:4620, 2001). (2) Immune Response: FLT3LG (fms-related tyrosine kinase 3 ligand) activates FLT3 and downstream pathways such as mTOR and RAS/MEK/ERK. It is reported to play an important role in regulating the immune response and is a gene associated with cancer (Kreiter et al., Cancer Res. 71:6132, 2011). TNFRSF14 (tumor necrosis factor receptor superfamily, member 14) also plays a role in regulating the immune response and is a known cancer related gene associated with lymphoma (Launay et al., Leukemia 26:559, 2012). (3) Cell Invasion and Migration: GLIPR2 (GLI pathogenesis-related 2) overexpression of this protein has been shown to promote migration and invasion via EMT in hepatocellular carcinoma (Huang et al., PLoS One 8:e77497, 2013). STMN3 (stathmin-like 3) has been found to stimulate proliferation, invasion and migration in cancer cell lines (Nair et al., Mol. Cancer 13:173, 2014). (4) WNT Signaling Pathway: WNT8A (wingless-type MMTV integration site family, member 8A) is a potent activator of the canonical WNT/β-catenin signaling pathway found to play a role in cancer progression (Merritt et al., BMC Cancer 9:378, 2009). Compound 107 translationally down regulated these select genes providing insight into possible mechanisms for how an MNK inhibitor can achieve therapeutic benefit in treating cancer.

TABLE 6
Gene Signature with Altered Translational Rate -
3 hr treatment with Compound 107 (10 μM)

| | | Log2 FC | |
|---|---|---|---|
| | | RPF(508)/ | RPF |
| ENSEMBL | HGNC | RPF(DMSO) | p-value |
| ENSG00000185271 | KLHL33 | −1.60103 | 0.004425 |
| ENSG00000179965 | ZNF771 | −1.59912 | 0.000476 |
| ENSG00000175745 | NR2F1 | −1.58646 | 0.005653 |
| ENSG00000134061 | CD180 | −1.41967 | 3.05E−15 |
| ENSG00000198339 | HIST1H4I | −1.41635 | 8.21E−11 |
| ENSG00000100055 | CYTH4 | −1.40783 | 1.02E−05 |
| ENSG00000147119 | CHST7 | −1.36995 | 0.000536 |
| ENSG00000048462 | TNFRSF17 | −1.36363 | 1.78E−12 |
| ENSG00000083290 | ULK2 | −1.32787 | 0.004012 |
| ENSG00000132530 | XAF1 | −1.28786 | 0.005338 |
| ENSG00000050344 | NFE2L3 | −1.27663 | 0.000693 |
| ENSG00000211677 | IGLC2 | −1.26774 | 0.005708 |
| ENSG00000115641 | FHL2 | −1.20655 | 6.50E−09 |
| ENSG00000140950 | KIAA1609 | −1.19879 | 0.000989 |
| ENSG00000185090 | MANEAL | −1.18289 | 0.002269 |
| ENSG00000119917 | IFIT3 | −1.1714 | 2.64E−07 |
| ENSG00000232810 | TNF | −1.153 | 0.00082 |
| ENSG00000167208 | SNX20 | −1.09911 | 0.007244 |
| ENSG00000156966 | B3GNT7 | −1.09193 | 0.002729 |
| ENSG00000171812 | COL8A2 | −1.09179 | 0.00164 |
| ENSG00000100628 | ASB2 | −1.08647 | 6.17E−07 |
| ENSG00000185215 | TNFAIP2 | −1.06679 | 0.002327 |
| ENSG00000043355 | ZIC2 | −1.05959 | 0.00449 |
| ENSG00000185361 | TNFAIP8L1 | −1.05304 | 0.004074 |
| ENSG00000143851 | PTPN7 | −1.0434 | 1.41E−05 |
| ENSG00000068137 | PLEKHH3 | −1.03706 | 0.001841 |
| ENSG00000176170 | SPHK1 | −1.02378 | 0.004102 |
| ENSG00000182319 | PRAGMIN.1 | −1.01718 | 1.18E−06 |
| ENSG00000172059 | KLF11 | −1.01017 | 0.007553 |
| ENSG00000164171 | ITGA2 | −1.0097 | 0.00134 |
| ENSG00000033327 | GAB2 | −1.00815 | 6.17E−09 |
| ENSG00000160712 | IL6R | −0.99939 | 0.004453 |
| ENSG00000152778 | IFIT5 | −0.99744 | 1.93E−06 |
| ENSG00000146232 | NFKBIE | −0.98337 | 5.97E−05 |
| ENSG00000168386 | FILIP1L | −0.98189 | 3.13E−07 |
| ENSG00000118513 | MYB | −0.97394 | 5.06E−08 |
| ENSG00000106351 | AGFG2 | −0.97226 | 1.66E−05 |
| ENSG00000112561 | TFEB | −0.96931 | 1.73E−06 |
| ENSG00000158473 | CD1D | −0.96447 | 0.0001 |
| ENSG00000122877 | EGR2 | −0.9562 | 0.008918 |
| ENSG00000033030 | ZCCHC8 | −0.95021 | 1.79E−07 |
| ENSG00000095370 | SH2D3C | −0.94928 | 2.11E−09 |
| ENSG00000175793 | SFN | −0.94169 | 1.83E−05 |
| ENSG00000114993 | RTKN | −0.93049 | 0.007109 |
| ENSG00000107554 | DNMBP | −0.92796 | 2.09E−05 |
| ENSG00000007968 | E2F2 | −0.91027 | 0.002424 |
| ENSG00000198807 | PAX9 | −0.90466 | 7.04E−07 |
| ENSG00000050730 | TNIP3 | −0.90457 | 0.000297 |
| ENSG00000011243 | AKAP8L | −0.90052 | 1.33E−08 |
| ENSG00000128011 | LRFN1 | −0.87462 | 0.001018 |
| ENSG00000126368 | NR1D1 | −0.86413 | 0.003281 |
| ENSG00000189007 | ADAT2 | −0.85883 | 0.00477 |
| ENSG00000169220 | RGS14 | −0.85752 | 0.002852 |
| ENSG00000112658 | SRF | −0.84127 | 2.46E−07 |
| ENSG00000141540 | TTYH2 | −0.82713 | 0.001188 |
| ENSG00000185745 | IFIT1 | −0.81979 | 0.000567 |
| ENSG00000174123 | TLR10 | −0.81508 | 4.00E−05 |
| ENSG00000155090 | KLF10 | −0.74898 | 0.00307 |
| ENSG00000188987 | HIST1H4D | −0.73374 | 1.21E−08 |
| ENSG00000196664 | TLR7 | −0.71303 | 0.000183 |
| ENSG00000112715 | VEGFA | −0.70624 | 0.001656 |
| ENSG00000128604 | IRF5 | −0.69732 | 1.09E−05 |
| ENSG00000137393 | RNF144B | 0.814234 | 2.12E−07 |
| ENSG00000243646 | IL10RB | 0.815546 | 0.008041 |
| ENSG00000022567 | SLC45A4 | 0.8358 | 2.19E−05 |
| ENSG00000113916 | BCL6 | 0.837293 | 6.26E−07 |
| ENSG00000166900 | STX3 | 0.839404 | 0.004292 |
| ENSG00000091317 | CMTM6 | 0.844015 | 7.82E−10 |
| ENSG00000198718 | FAM179B | 0.844391 | 0.000204 |
| ENSG00000204256 | BRD2 | 0.846845 | 1.14E−10 |
| ENSG00000175040 | CHST2 | 0.852537 | 4.39E−05 |
| ENSG00000144468 | RHBDD1 | 0.868301 | 0.001005 |
| ENSG00000132334 | PTPRE | 0.872291 | 9.77E−10 |
| ENSG00000197872 | FAM49A | 0.894748 | 1.32E−06 |
| ENSG00000196352 | CD55 | 0.903985 | 5.51E−08 |
| ENSG00000132406 | TMEM128 | 0.907408 | 0.003367 |
| ENSG00000175536 | LIPT2 | 0.938936 | 0.004759 |
| ENSG00000162924 | REL | 0.94092 | 3.83E−14 |
| ENSG00000065989 | PDE4A | 0.945142 | 0.005854 |
| ENSG00000130775 | C1orf38 | 0.962788 | 5.15E−06 |
| ENSG00000173011 | TADA2B | 0.96342 | 0.002327 |
| ENSG00000113532 | ST8SIA4 | 0.971872 | 1.30E−07 |
| ENSG00000198551 | ZNF627 | 0.982826 | 0.000151 |
| ENSG00000163508 | EOMES | 0.993677 | 0.004542 |
| ENSG00000132329 | RAMP1 | 1.015089 | 4.40E−05 |
| ENSG00000172086 | KRCC1 | 1.015193 | 0.000305 |
| ENSG00000229644 | NAMPTL | 1.022118 | 0.002546 |
| ENSG00000095794 | CREM | 1.027472 | 3.18E−10 |
| ENSG00000130449 | ZSWIM6 | 1.028861 | 1.24E−06 |
| ENSG00000135114 | OASL | 1.03488 | 0.005831 |
| ENSG00000143669 | LYST | 1.059095 | 0.005706 |
| ENSG00000178764 | ZHX2 | 1.071475 | 1.28E−07 |
| ENSG00000114013 | CD86 | 1.099711 | 2.65E−17 |
| ENSG00000013441 | CLK1 | 1.110988 | 5.96E−12 |
| ENSG00000157933 | SKI | 1.13623 | 0.000237 |
| ENSG00000134460 | IL2RA | 1.138597 | 4.30E−05 |
| ENSG00000113240 | CLK4 | 1.152334 | 1.07E−05 |
| ENSG00000144847 | IGSF11 | 1.180317 | 6.74E−07 |
| ENSG00000188177 | ZC3H6 | 1.19016 | 0.002659 |
| ENSG00000113369 | ARRDC3 | 1.220686 | 0.000349 |
| ENSG00000104951 | IL4I1 | 1.253109 | 3.36E−24 |
| ENSG00000139926 | FRMD6 | 1.263872 | 0.002762 |
| ENSG00000188389 | PDCD1 | 1.27866 | 4.28E−06 |
| ENSG00000141655 | TNFRSF11A | 1.292303 | 1.01E−07 |
| ENSG00000170525 | PFKFB3 | 1.305374 | 6.19E−21 |
| ENSG00000166046 | TCP11L2 | 1.317384 | 2.86E−18 |
| ENSG00000120875 | DUSP4 | 1.355674 | 0.00018 |
| ENSG00000126353 | CCR7 | 1.411188 | 2.77E−26 |
| ENSG00000188042 | ARL4C | 1.463755 | 1.68E−10 |
| ENSG00000184588 | PDE4B | 1.520963 | 3.17E−18 |
| ENSG00000102755 | FLT1 | 1.527831 | 7.56E−05 |
| ENSG00000159788 | RGS12 | 1.753404 | 0.001162 |
| ENSG00000116741 | RGS2 | 1.766509 | 2.99E−16 |
| ENSG00000165730 | STOX1 | 1.780548 | 0.003315 |
| ENSG00000100867 | DHRS2 | 1.785294 | 0.000326 |
| ENSG00000164849 | GPR146 | 1.968529 | 0.0083 |
| ENSG00000119508 | NR4A3 | 2.124319 | 4.05E−12 |
| ENSG00000090104 | RGS1 | 2.164612 | 0.000101 |
| ENSG00000204947 | ZNF425 | 2.528277 | 3.49E−05 |
| ENSG00000136244 | IL6 | 2.604373 | 1.24E−11 |
| ENSG00000132872 | SYT4 | 2.6084 | 4.65E−14 |
| ENSG00000161835 | GRASP | 2.81282 | 2.88E−21 |
| ENSG00000186081 | KRT5 | 3.282757 | 1.16E−07 |
| ENSG00000138378 | STAT4 | 3.568756 | 9.08E−05 |
| ENSG00000123358 | NR4A1 | 3.727382 | 3.44E−10 |
| ENSG00000183508 | FAM46C | 4.414207 | 2.21E−50 |

### Conclusion

Ribosome profiling identified an MM(regulon that is strongly connected to regulating immune and inflammatory responsive and regulatory genes. This is consistent with previous reports that MNK kinases regulate pro-inflammatory cytokines. These findings are significant as pro-inflammatory cytokines are known mediators of tumor-stromal cell recruitment and interaction. Pro-inflammatory cytokines are drivers of key hallmarks of cancer including angiogenesis, migration and invasion, and immune evasion, while also driving drug resistance. Select genes within the MNK regulon identified by treatment of TMD8 with Compound 107 have also been observed to be modulated in additional systems. Compound 107 treatment of the DLBCL cell line, TMD8, demonstrated modulation of one or more of the cytokines evaluated (TNFα, IL6, IL10). Likewise, Compound 107 treatment of CD3/CD28 activated T cells resulted in the reduction of cell surface levels of the immune checkpoint inhibitors PD-1 and LAG-3 along with reduction in select cytokines/chemokines (TNFα, IL10, CXCL10). The observed overlap of select genes regulated between multiple model systems suggests an element of commonality for MNK regulation.

### Example 2

### 5′ AND 3′ Recognition Elements of MNK Sensitive Genes

Identification of de novo 3′- and 5′-UTR recognition elements in the MNK-sensitive genes was conducted using the DREME motif identification algorithm. The UTRs were searched for 5-12 mers that are enriched compared to the UTRs from the whole genome. Enrichment in either the 3′- or 5′-UTRs of MNK-sensitive genes was evaluated using bootstrap analysis.

### Identification of 5′-UTR Recognition Elements in Translationally Regulated Geneset

A recent study identified 5′ untranslated region (5′-UTR) regulatory elements in the mouse genome that rendered select transcripts sensitive to expression levels of eIF4E. These transcripts were found to contain a cytosine rich 15-nucleotide motif termed the cytosine-enriched regulator of translation (CERT) domain in the 5′-UTR. 70% of the targets sensitive to eIF4E levels were found to be enriched for this CERT sequence (Truitt et al., Cell 162:1, 2015).

Only a small subset of genes exhibited modulation in translational efficiency (ribosome occupancy changes in the absence of modulation of total mRNA) indicating that MNK inhibition of the phosphorylation of eIF4E regulates the translation of a select set of genes. This is consistent with reports that the translational machinery can discriminate between different mRNA transcripts. The sequence and structural features of the 5′-UTR are suggested to play a role in regulating the efficiency of translation. The 5′-UTR of the Compound 107 sensitive genes treated with 10 μM Compound 107 for 3 hr were evaluated for length and percentage GC content. The length of the 5′-UTR was significantly longer relative to the whole genome suggesting that these transcripts are sensitive to regulating eIF4E activity (Table 7); however, the percent GC content was not statistically different relative to the background.

TABLE 7
Characteristics of the 5′-UTR of Compound 107 Sensitive Genes

| | 5′-UTR MNK | | |
|---|---|---|---|
| Sample | Sensitive Genes | Background | p-value |
| % GC Content | 66.395 | 62.46 | 0.02 |
| 5′-UTR Length | 365.34 | 238.637 | 0.01 |

To determine if additional features within the 5′-UTR sensitize them to inhibition of MNK, the MNK translational efficiency sensitive genes were evaluated for sequence specific motifs to identify potential cis-acting regulatory elements. An unbiased search using DREME, a motif discovery algorithm, was utilized to identify de novo recognition elements. This search identified three sequence specific 5′-UTR motifs that were statistically enriched relative to the entire genome (Table 8): a cytosine-rich 9-mer ([C(C|U)(C|U)(C|G)CCC(G|U)(C|G)]; p-value 2.78×10⁻⁶) and 7- and 6-mer guanine-rich motifs ([GGGGC(C|U)C]; p-value 4.94×10⁻⁸) and ([GCCGG(C1U)]; p-value 9.76×10⁻⁸), respectively. Over 80% of the genes regulated at the translational efficiency level by MNK inhibition contained one or more of the three distinct 5′-UTR recognition motifs. 55% of the genes contained the 9-mer cytosine-rich element, and 51% and 62% contained the 7-mer and 6-mer guanine-rich motif, respectively. The majority of genes containing the 9-mer and 6-mer 5′-UTR motifs contained multiple cis-acting recognition elements with 62% of the 9-mer and 79% of the 6-mer genes containing more than one of the motifs. Table 9 lists which translationally regulated genes contain the 5′-UTR recognition motifs and the number of occurrences of each motif within the 5′-UTR of specific genes.

The MNK sensitive translationally regulated genes containing the 5′-UTR cis-acting motifs have been reported to play a role in cancer development. Gene Ontology analysis determined that the MNK sensitive genes are associated with the immune and inflammatory response, response to stimulus, post-translational modification, cell invasion and migration, and WNT signaling pathway biological functional categories.

TABLE 8
De novo 5'-UTR recognition motifs sensitive to Compound 107 inhibition
of MNK and their enrichment within the Compound 107 gene sets

| | | | % Genes | % Genes |
|---|---|---|---|---|
| | | | with Motif - | with Motif - |
| | De Novo 5′-UTR Recognition | | Translational | Translational |
| Motif | Motifs | p-value | Efficiency | Rate |
| 9-mer | C(C\|U)(C\|U)(G\|C)CCC(G\|U)(G\|C) | 2.78 × 10⁻⁶ | 55 | 10 |
| 7-mer | GGGGC(C\|U)C | 4.94 × 10⁻⁸ | 51 | 8 |
| 6-mer | GCCGG(C\|U) | 9.76 × 10⁻⁸ | 62 | 9 |

TABLE 9
Occurrence of de novo 5′-UTR recognition elements identified in
the MNK-sensitive gene set regulated at translational efficiency
after incubation of TMD8 cells with 10 μM Compound 107 for 3 hours

| | | 9-mer | 7-mer | 6-mer |
|---|---|---|---|---|
| | | # of | # of | # of |
| | | Occur- | Occur- | Occur- |
| Ensembl ID | HGNC | rences | rences | rences |
| ENSG00000175745 | NR2F1 | 7 | 1 | 14 |
| ENSG00000147852 | VLDLR | 5 | 0 | 0 |
| ENSG00000172375 | C2CD2L | 5 | 4 | 5 |
| ENSG00000186174 | BCL9L | 5 | 4 | 2 |
| ENSG00000105971 | CAV2 | 3 | 2 | 2 |
| ENSG00000110881 | ACCN2 | 3 | 1 | 4 |
| ENSG00000163251 | FZD5 | 3 | 1 | 1 |
| ENSG00000171174 | RBKS | 3 | 1 | 1 |
| ENSG00000083290 | ULK2 | 2 | 1 | 2 |
| ENSG00000102554 | KLF5 | 2 | 2 | 4 |
| ENSG00000119138 | KLF9 | 2 | 0 | 9 |
| ENSG00000132872 | SYT4 | 2 | 0 | 2 |
| ENSG00000154620 | TMSB4Y | 2 | 1 | 2 |
| ENSG00000157933 | SKI | 2 | 1 | 0 |
| ENSG00000177946 | CENPBD1 | 2 | 3 | 2 |
| ENSG00000184574 | LPAR5 | 2 | 1 | 0 |
| ENSG00000008513 | ST3GAL1 | 1 | 0 | 2 |
| ENSG00000061492 | WNT8A | 1 | 0 | 1 |
| ENSG00000112290 | WASF1 | 1 | 2 | 1 |
| ENSG00000156966 | B3GNT7 | 1 | 0 | 4 |
| ENSG00000157873 | TNFRSF14 | 1 | 1 | 2 |
| ENSG00000162738 | VANGL2 | 1 | 2 | 1 |
| ENSG00000179965 | ZNF771 | 1 | 0 | 3 |
| ENSG00000198208 | RPS6KL1 | 1 | 0 | 2 |
| ENSG00000204947 | ZNF425 | 1 | 0 | 3 |
| ENSG00000205476 | CCDC85C | 1 | 1 | 2 |
| ENSG00000049246 | PER3 | 0 | 1 | 0 |
| ENSG00000058335 | RASGRF1 | 0 | 1 | 0 |
| ENSG00000078401 | EDN1 | 0 | 0 | 0 |
| ENSG00000090554 | FLT3LG | 0 | 2 | 0 |
| ENSG00000102100 | SLC35A2 | 0 | 0 | 0 |
| ENSG00000119508 | NR4A3 | 0 | 0 | 0 |
| ENSG00000122694 | GLIPR2 | 0 | 0 | 2 |
| ENSG00000125449 | ARMC7 | 0 | 1 | 0 |
| ENSG00000132825 | PPP1R3D | 0 | 0 | 2 |
| ENSG00000134222 | PSRC1 | 0 | 1 | 0 |
| ENSG00000135426 | KIAA0748 | 0 | 0 | 0 |
| ENSG00000139718 | SETD1B | 0 | 0 | 2 |
| ENSG00000141526 | SLC16A3 | 0 | 0 | 1 |
| ENSG00000142961 | MOB3C | 0 | 0 | 0 |
| ENSG00000145685 | LHFPL2 | 0 | 0 | 2 |
| ENSG00000175764 | TTLL11 | 0 | 1 | 0 |
| ENSG00000184226 | PCDH9 | 0 | 0 | 0 |
| ENSG00000197457 | STMN3 | 0 | 0 | 0 |
| ENSG00000197852 | FAM212B | 0 | 0 | 2 |
| ENSG00000203778 | C6orf225 | 0 | 0 | 0 |
| ENSG00000205571 | SMN2 | 0 | 2 | 0 |

Inhibition of MNK1/2 resulted in the statistically significant modulation of translation rate or transcript levels for a small subset of genes after treatment of TMD8 cells with 1004 Compound 107 for 48 hours. These genes were evaluated for the presence of the 5′-UTR cis-acting elements identified from the gene set where treatment with Compound 107 resulted in modulation of the translational efficiency. Table 10 lists the presence of the 5′-UTR motifs for the genes regulated by Compound 107 at the translational rate. Only approximately 10% of the genes contained the recognition elements suggesting that these sequence motifs are indeed a recognition element involved in regulating translation initiation (see Table 8).

Genes that did contain the 5′-UTR recognition motifs were enriched in immune related biological functional categories including immune and inflammatory responses, defense and stress responses and cytokine mediated signaling. In addition, the genes that did contain the recognition elements were also predominately regulated at the translational efficiency level where drug treatment caused a substantial reduction in ribosome occupancy with minimal changes in total mRNA (e.g., CD97, IRF7, STATSA, WNT8A), further supporting the role of these cis-acting elements in regulating translation initiation.

TABLE 10
Occurrence of de novo 5′-UTR recognition elements in the
MNK-sensitive gene set regulated at translational rate after
incubation of TMD8 cells with 10 μM Compound 107 for 48 hours

| | | 9-mer | 7-mer | 6-mer |
|---|---|---|---|---|
| | | # of | # of | # of |
| | | Occur- | Occur- | Occur- |
| Ensembl ID | HGNC | rences | rences | rences |
| ENSG00000163435 | ELF3 | 5 | 3 | 3 |
| ENSG00000086062 | B4GALT1 | 3 | 0 | 0 |
| ENSG00000086730 | LAT2 | 3 | 0 | 0 |
| ENSG00000131650 | KREMEN2 | 3 | 0 | 1 |
| ENSG00000135363 | LMO2 | 3 | 1 | 2 |
| ENSG00000173511 | VEGFB | 3 | 1 | 1 |
| ENSG00000175274 | TP53I11 | 3 | 0 | 1 |
| ENSG00000197747 | S100A10 | 3 | 0 | 1 |
| ENSG00000197956 | S100A6 | 3 | 0 | 1 |
| ENSG00000078900 | TP73 | 2 | 1 | 2 |
| ENSG00000112297 | AIM1 | 2 | 3 | 1 |
| ENSG00000120915 | EPHX2 | 2 | 0 | 0 |
| ENSG00000132872 | SYT4 | 2 | 0 | 2 |
| ENSG00000136048 | DRAM1 | 2 | 0 | 0 |
| ENSG00000138152 | BTBD16 | 2 | 1 | 0 |
| ENSG00000143545 | RAB13 | 2 | 0 | 0 |
| ENSG00000144115 | THNSL2 | 2 | 2 | 0 |
| ENSG00000157613 | CREB3L1 | 2 | 3 | 4 |
| ENSG00000162419 | GMEB1 | 2 | 0 | 0 |
| ENSG00000166165 | CKB | 2 | 0 | 0 |
| ENSG00000177169 | ULK1 | 2 | 0 | 0 |
| ENSG00000180879 | SSR4 | 2 | 5 | 5 |
| ENSG00000182197 | EXT1 | 2 | 1 | 6 |
| ENSG00000182319 | PRAGMIN.1 | 2 | 0 | 0 |
| ENSG00000185507 | IRF7 | 2 | 0 | 2 |
| ENSG00000011523 | CEP68 | 1 | 0 | 0 |
| ENSG00000038427 | VCAN | 1 | 0 | 0 |
| ENSG00000039068 | CDH1 | 1 | 1 | 0 |
| ENSG00000061492 | WNT8A | 1 | 0 | 1 |
| ENSG00000069956 | MAPK6 | 1 | 1 | 0 |
| ENSG00000075673 | ATP12A | 1 | 0 | 3 |
| ENSG00000091490 | SEL1L3 | 1 | 0 | 3 |
| ENSG00000100097 | LGALS1 | 1 | 1 | 0 |
| ENSG00000100385 | IL2RB | 1 | 0 | 0 |
| ENSG00000103316 | CRYM | 1 | 0 | 0 |
| ENSG00000111331 | OAS3 | 1 | 0 | 0 |
| ENSG00000111679 | PTPN6 | 1 | 0 | 2 |
| ENSG00000117115 | PADI2 | 1 | 0 | 0 |
| ENSG00000122547 | EEPD1 | 1 | 1 | 1 |
| ENSG00000123096 | SSPN | 1 | 0 | 0 |
| ENSG00000126561 | STAT5A | 1 | 1 | 4 |
| ENSG00000137628 | DDX60 | 1 | 0 | 0 |
| ENSG00000142227 | EMP3 | 1 | 0 | 0 |
| ENSG00000143554 | SLC27A3 | 1 | 0 | 0 |
| ENSG00000143851 | PTPN7 | 1 | 0 | 1 |
| ENSG00000148671 | C10orf116 | 1 | 0 | 0 |
| ENSG00000155367 | PPM1J | 1 | 0 | 3 |
| ENSG00000159403 | C1R | 1 | 0 | 0 |
| ENSG00000160190 | SLC37A1 | 1 | 0 | 0 |
| ENSG00000160193 | WDR4 | 1 | 0 | 0 |
| ENSG00000162772 | ATF3 | 1 | 0 | 4 |
| ENSG00000162849 | KIF26B | 1 | 0 | 0 |
| ENSG00000163545 | NUAK2 | 1 | 0 | 0 |
| ENSG00000165795 | NDRG2 | 1 | 0 | 0 |
| ENSG00000166016 | ABTB2 | 1 | 2 | 2 |
| ENSG00000166145 | SPINT1 | 1 | 1 | 0 |
| ENSG00000167930 | ITFG3 | 1 | 0 | 0 |
| ENSG00000168679 | SLC16A4 | 1 | 0 | 0 |
| ENSG00000168952 | STXBP6 | 1 | 1 | 4 |
| ENSG00000170801 | HTRA3 | 1 | 0 | 5 |
| ENSG00000170873 | MTSS1 | 1 | 1 | 0 |
| ENSG00000173890 | GPR160 | 1 | 0 | 2 |
| ENSG00000183508 | FAM46C | 1 | 0 | 0 |
| ENSG00000198734 | F5 | 1 | 0 | 0 |
| ENSG00000213983 | AP1G2 | 1 | 1 | 1 |
| ENSG00000232810 | TNF | 1 | 0 | 0 |
| ENSG00000005379 | BZRAP1 | 0 | 2 | 0 |
| ENSG00000007255 | TRAPPC6A | 0 | 0 | 0 |
| ENSG00000011600 | TYROBP | 0 | 0 | 0 |
| ENSG00000011638 | TMEM159 | 0 | 0 | 0 |
| ENSG00000033327 | GAB2 | 0 | 1 | 4 |
| ENSG00000040933 | INPP4A | 0 | 0 | 0 |
| ENSG00000047346 | FAM214A | 0 | 0 | 0 |
| ENSG00000050344 | NFE2L3 | 0 | 0 | 4 |
| ENSG00000050730 | TNIP3 | 0 | 0 | 0 |
| ENSG00000054219 | LY75 | 0 | 0 | 0 |
| ENSG00000064687 | ABCA7 | 0 | 1 | 0 |
| ENSG00000068650 | ATP11A | 0 | 0 | 0 |
| ENSG00000069424 | KCNAB2 | 0 | 0 | 0 |
| ENSG00000070190 | DAPP1 | 0 | 0 | 0 |
| ENSG00000073737 | DHRS9 | 0 | 0 | 0 |
| ENSG00000075643 | MOCOS | 0 | 0 | 0 |
| ENSG00000076706 | MCAM | 0 | 0 | 0 |
| ENSG00000080493 | SLC4A4 | 0 | 0 | 0 |
| ENSG00000085733 | CTTN | 0 | 0 | 0 |
| ENSG00000089127 | OAS1 | 0 | 0 | 0 |
| ENSG00000089327 | FXYD5 | 0 | 0 | 2 |
| ENSG00000089692 | LAG3 | 0 | 0 | 0 |
| ENSG00000090104 | RGS1 | 0 | 0 | 0 |
| ENSG00000099377 | HSD3B7 | 0 | 1 | 2 |
| ENSG00000100239 | PPP6R2 | 0 | 0 | 0 |
| ENSG00000100300 | TSPO | 0 | 0 | 0 |
| ENSG00000100422 | CERK | 0 | 0 | 0 |
| ENSG00000100628 | ASB2 | 0 | 0 | 0 |
| ENSG00000101190 | TCFL5 | 0 | 0 | 0 |
| ENSG00000102181 | CD99L2 | 0 | 2 | 0 |
| ENSG00000102962 | CCL22 | 0 | 0 | 0 |
| ENSG00000104381 | GDAP1 | 0 | 0 | 0 |
| ENSG00000104974 | LILRA1 | 0 | 0 | 0 |
| ENSG00000105246 | EBI3 | 0 | 0 | 0 |
| ENSG00000105339 | DENND3 | 0 | 0 | 0 |
| ENSG00000105501 | SIGLEC5 | 0 | 1 | 0 |
| ENSG00000106211 | HSPB1 | 0 | 0 | 0 |
| ENSG00000108469 | RECQL5 | 0 | 0 | 1 |
| ENSG00000109323 | MANBA | 0 | 0 | 1 |
| ENSG00000109654 | TRIM2 | 0 | 0 | 0 |
| ENSG00000111335 | OAS2 | 0 | 0 | 2 |
| ENSG00000111863 | C6orf105 | 0 | 0 | 0 |
| ENSG00000112116 | IL17F | 0 | 0 | 0 |
| ENSG00000112799 | LY86 | 0 | 2 | 0 |
| ENSG00000113302 | IL12B | 0 | 0 | 0 |
| ENSG00000115155 | OTOF | 0 | 0 | 1 |
| ENSG00000115415 | STAT1 | 0 | 1 | 0 |
| ENSG00000115902 | SLC1A4 | 0 | 0 | 2 |
| ENSG00000116701 | NCF2 | 0 | 0 | 0 |
| ENSG00000116741 | RGS2 | 0 | 1 | 0 |
| ENSG00000117228 | GBP1 | 0 | 0 | 0 |
| ENSG00000118785 | SPP1 | 0 | 0 | 0 |
| ENSG00000119139 | TJP2 | 0 | 0 | 0 |
| ENSG00000119698 | PPP4R4 | 0 | 0 | 0 |
| ENSG00000119801 | YPEL5 | 0 | 0 | 0 |
| ENSG00000119917 | IFIT3 | 0 | 0 | 0 |
| ENSG00000120756 | PLS1 | 0 | 0 | 0 |
| ENSG00000121858 | TNFSF10 | 0 | 0 | 1 |
| ENSG00000123146 | CD97 | 0 | 0 | 2 |
| ENSG00000125089 | SH3TC1 | 0 | 0 | 0 |
| ENSG00000126259 | KIRREL2 | 0 | 1 | 0 |
| ENSG00000126353 | CCR7 | 0 | 0 | 0 |
| ENSG00000126709 | IFI6 | 0 | 0 | 0 |
| ENSG00000127838 | PNKD | 0 | 0 | 0 |
| ENSG00000129226 | CD68 | 0 | 0 | 0 |
| ENSG00000130589 | RP4-697K14.7.1 | 0 | 0 | 0 |
| ENSG00000132530 | XAF1 | 0 | 0 | 0 |
| ENSG00000132622 | HSPA12B | 0 | 0 | 0 |
| ENSG00000133106 | EPSTI1 | 0 | 0 | 0 |
| ENSG00000135114 | OASL | 0 | 0 | 0 |
| ENSG00000135245 | HILPDA | 0 | 0 | 0 |
| ENSG00000135678 | CPM | 0 | 0 | 0 |
| ENSG00000136197 | C7orf25 | 0 | 0 | 0 |
| ENSG00000136244 | IL6 | 0 | 0 | 0 |
| ENSG00000136634 | IL10 | 0 | 0 | 0 |
| ENSG00000136816 | TOR1B | 0 | 0 | 0 |
| ENSG00000137522 | RNF121 | 0 | 0 | 0 |
| ENSG00000137642 | SORL1 | 0 | 1 | 0 |
| ENSG00000137880 | GCHFR | 0 | 1 | 0 |
| ENSG00000137959 | IFI44L | 0 | 0 | 0 |
| ENSG00000138642 | HERC6 | 0 | 0 | 0 |
| ENSG00000141655 | TNFRSF11A | 0 | 0 | 0 |
| ENSG00000142197 | DOPEY2 | 0 | 0 | 0 |
| ENSG00000142494 | SLC47A1 | 0 | 0 | 2 |
| ENSG00000144218 | AFF3 | 0 | 0 | 0 |
| ENSG00000146072 | TNFRSF21 | 0 | 0 | 0 |
| ENSG00000147813 | NAPRT1 | 0 | 0 | 0 |
| ENSG00000148450 | MSRB2 | 0 | 0 | 0 |
| ENSG00000148814 | LRRC27 | 0 | 1 | 0 |
| ENSG00000149289 | ZC3H12C | 0 | 0 | 0 |
| ENSG00000152778 | IFIT5 | 0 | 0 | 0 |
| ENSG00000155008 | APOOL | 0 | 0 | 0 |
| ENSG00000157601 | MX1 | 0 | 0 | 5 |
| ENSG00000160307 | S100B | 0 | 0 | 0 |
| ENSG00000161642 | ZNF385A | 0 | 0 | 0 |
| ENSG00000162896 | PIGR | 0 | 0 | 0 |
| ENSG00000165949 | IFI27 | 0 | 0 | 0 |
| ENSG00000166825 | ANPEP | 0 | 1 | 0 |
| ENSG00000168298 | HIST1H1E | 0 | 0 | 0 |
| ENSG00000168961 | LGALS9 | 0 | 0 | 2 |
| ENSG00000169245 | CXCL10 | 0 | 0 | 0 |
| ENSG00000170054 | SERPINA9 | 0 | 0 | 0 |
| ENSG00000172578 | KLHL6 | 0 | 0 | 0 |
| ENSG00000175793 | SFN | 0 | 0 | 0 |
| ENSG00000175866 | BAIAP2 | 0 | 0 | 0 |
| ENSG00000176058 | TPRN | 0 | 0 | 0 |
| ENSG00000177082 | WDR73 | 0 | 0 | 0 |
| ENSG00000178498 | DTX3 | 0 | 0 | 0 |
| ENSG00000178567 | EPM2AIP1 | 0 | 0 | 0 |
| ENSG00000181104 | F2R | 0 | 0 | 1 |
| ENSG00000181192 | DHTKD1 | 0 | 0 | 1 |
| ENSG00000182150 | C9orf102 | 0 | 0 | 4 |
| ENSG00000184588 | PDE4B | 0 | 0 | 0 |
| ENSG00000184979 | USP18 | 0 | 0 | 2 |
| ENSG00000185201 | IFITM2 | 0 | 0 | 0 |
| ENSG00000185291 | IL3RA | 0 | 2 | 0 |
| ENSG00000185745 | IFIT1 | 0 | 0 | 0 |
| ENSG00000185885 | IFITM1 | 0 | 0 | 0 |
| ENSG00000187608 | ISG15 | 0 | 0 | 3 |
| ENSG00000188095 | MESP2 | 0 | 0 | 0 |
| ENSG00000188987 | HIST1H4D | 0 | 0 | 2 |
| ENSG00000196154 | S100A4 | 0 | 0 | 0 |
| ENSG00000196226 | HIST1H2BB | 0 | 0 | 0 |
| ENSG00000196374 | HIST1H2BM | 0 | 0 | 0 |
| ENSG00000196433 | ASMT | 0 | 0 | 0 |
| ENSG00000196586 | MYO6 | 0 | 0 | 2 |
| ENSG00000196735 | HLA-DQA1 | 0 | 0 | 0 |
| ENSG00000197121 | PGAP1 | 0 | 2 | 0 |
| ENSG00000197355 | UAP1L1 | 0 | 0 | 0 |
| ENSG00000197409 | HIST1H3D | 0 | 0 | 0 |
| ENSG00000197471 | SPN | 0 | 0 | 0 |
| ENSG00000197594 | ENPP1 | 0 | 0 | 0 |
| ENSG00000198339 | HIST1H4I | 0 | 0 | 0 |
| ENSG00000198374 | HIST1H2AL | 0 | 0 | 0 |
| ENSG00000198682 | PAPSS2 | 0 | 0 | 0 |
| ENSG00000198799 | LRIG2 | 0 | 0 | 0 |
| ENSG00000198959 | TGM2 | 0 | 2 | 0 |
| ENSG00000203813 | HIST1H3H | 0 | 0 | 0 |
| ENSG00000204186 | ZDBF2 | 0 | 1 | 0 |
| ENSG00000204388 | HSPA1B | 0 | 0 | 2 |
| ENSG00000204580 | DDR1 | 0 | 0 | 0 |
| ENSG00000237541 | HLA-DQA2 | 0 | 0 | 0 |
| ENSG00000244509 | APOBEC3C | 0 | 1 | 0 |
| ENSG00000256018 | HIST1H3G | 0 | 0 | 0 |
| ENSG00000256043 | CTSO | 0 | 0 | 0 |
| ENSG00000259207 | ITGB3 | 0 | 0 | 0 |

### Identification of 3′-UTR Recognition Elements in Translationally Regulated Geneset

mRNA stability is recognized as an important post-translational mechanism controlling the expression of a larger number of genes. Transcript stability is regulated by cis-acting elements localized in the 3′-untranslated region (3′-UTR) and trans-acting factors such as microRNAs and RNA-binding proteins. The best characterized cis-acting sequence is the AU-rich element that is reported to contain sequence recognition elements that control mRNA stability or translation. AU-rich elements (AREs) found in the 3′-UTR of many mRNAs provide recognition sites for binding proteins. HNRNPA1 is an ARE binding protein that is reportedly phosphorylated by MNK and has been shown to regulate the stability of TNFα (Buxade et al., Immunity 23:177, 2005).

The MNK-sensitive genes identified from ribosome profiling that were modulated at their translational rate were evaluated for the presence of the AUUUA ARE recognition sequence in their 3′-UTRs. The majority of genes (>65%) were found to contain the AUUUA recognition element; however, this is only slightly enriched above background. Closer analysis identified genes that contained multiple ARE recognition sites within their 3′-UTR that were separated by <15 nucleotides. Approximately 30% of the MNK-sensitive genes contain multiple ARE sites. Many of these genes were found to be associated with the immune, inflammatory or defense response functional classifications (e.g., TNFα, IL6, IL12B, ENPP1, F2R, LY75 and NUAK2).

The genes regulated at the translational rate after treatment of TMD8 cells with 10 μM Compound 107 for 48 hours were also evaluated for sequence specific motifs in their 3′-UTRs. An unbiased search using DREME, a motif discovery algorithm, was utilized to identify de novo recognition elements. This search identified three unique sequence specific 3′-UTR motifs that were statistically enriched relative to the entire background as determined by bootstrap analysis (Table 11). Two 7-mer motifs ([GGA(G|U)U(G|C)C], p-value 2.39×10⁻⁶) and ([CC(A|G)UUCC], p-value 1.55×10′), and a 10-mer ([CCCAA(A|C)UCCC], p-value 1.09×10⁻⁷). All three contain a common signature sequence [A(A/U)UCC] within the identified 3′-UTR motifs that is unique with respect to the AUUUA ARE element. 60% of the genes contain one or more of the three 3′-UTR recognition motifs. 48% of the genes contain the GGA(G|U)U(G|C)C 7-mer motif, 31% contain the second 7-mer motif, CC(A|G)UUCC, and only 7% of the genes contain the 10-mer motif. Essentially all of the genes containing the 10-mer motif also contain one of the 7-mer motifs. The occurrence of each 3′-UTR recognition motif is listed in Table 12 for genes modulated at the translational rate with treatment with an MNK inhibitor.

The genes containing the 3′-UTR recognition elements are enriched for the immune/inflammatory regulation and response, defense and stress response, and cytokine mediated signaling biological functional categories. Approximately 50% of the genes containing either 7-mer 3′-UTR motif and essentially all of the genes containing the 10-mer recognition element were enriched within these immune related functional classifications.

TABLE 11
De novo 3′-UTR recognition motifs sensitive to Compound 107 inhibition
of MNK and their enrichment within the Compound 107 gene sets

| | De Novo 3′-UTR Recognition | | % Genes with Motif - |
|---|---|---|---|
| Motif | Motifs | p-value | Translational Rate |
| 7-mer A | GGA(G\|U)U(G\|C)C | 2.39 × 10⁻⁶ | 48 |
| 7-mer B | CC(A\|G)UUCC | 1.55 × 10⁻⁶ | 31 |
| 10-mer | CCCAA(A\|C)UCCC | 1.09 × 10⁻⁷ | 7 |

TABLE 12
Occurrence of de novo 3′-UTR recognition elements identified in
the MNK-sensitive gene set regulated at the translational rate after
incubation of TMD8 cells with 10 μM Compound 107 for 48 hours

| | | 7-mer_A | 7-mer_B | 10-mer |
|---|---|---|---|---|
| | | # of | # of | # of |
| | | Occur- | Occur- | Occur- |
| Ensembl ID | HGNC | rences | rences | rences |
| ENSG00000148814 | LRRC27 | 14 | 1 | 0 |
| ENSG00000198799 | LRIG2 | 9 | 1 | 0 |
| ENSG00000197121 | PGAP1 | 6 | 0 | 0 |
| ENSG00000135678 | CPM | 5 | 0 | 0 |
| ENSG00000111331 | OAS3 | 4 | 2 | 1 |
| ENSG00000132530 | XAF1 | 4 | 1 | 0 |
| ENSG00000136816 | TOR1B | 4 | 0 | 0 |
| ENSG00000137642 | SORL1 | 4 | 1 | 0 |
| ENSG00000143851 | PTPN7 | 4 | 0 | 0 |
| ENSG00000172578 | KLHL6 | 4 | 0 | 0 |
| ENSG00000102962 | CCL22 | 3 | 1 | 2 |
| ENSG00000100385 | IL2RB | 3 | 0 | 1 |
| ENSG00000197594 | ENPP1 | 3 | 1 | 1 |
| ENSG00000177169 | ULK1 | 3 | 0 | 0 |
| ENSG00000063587 | ZNF275 | 3 | 0 | 0 |
| ENSG00000100422 | CERK | 3 | 2 | 0 |
| ENSG00000141655 | TNFRSF11A | 3 | 0 | 0 |
| ENSG00000196586 | MYO6 | 3 | 2 | 0 |
| ENSG00000086062 | B4GALT1 | 2 | 1 | 1 |
| ENSG00000033327 | GAB2 | 2 | 4 | 1 |
| ENSG00000039068 | CDH1 | 2 | 1 | 1 |
| ENSG00000166016 | ABTB2 | 2 | 1 | 1 |
| ENSG00000182197 | EXT1 | 2 | 0 | 0 |
| ENSG00000183508 | FAM46C | 2 | 2 | 0 |
| ENSG00000007255 | TRAPPC6A | 2 | 0 | 0 |
| ENSG00000011523 | CEP68 | 2 | 2 | 0 |
| ENSG00000073737 | DHRS9 | 2 | 1 | 0 |
| ENSG00000075643 | MOCOS | 2 | 0 | 0 |
| ENSG00000078900 | TP73 | 2 | 2 | 0 |
| ENSG00000080493 | SLC4A4 | 2 | 1 | 0 |
| ENSG00000091490 | SEL1L3 | 2 | 0 | 0 |
| ENSG00000099377 | HSD3B7 | 2 | 0 | 0 |
| ENSG00000101190 | TCFL5 | 2 | 1 | 0 |
| ENSG00000112297 | AIM1 | 2 | 0 | 0 |
| ENSG00000119139 | TJP2 | 2 | 0 | 0 |
| ENSG00000119917 | IFIT3 | 2 | 1 | 0 |
| ENSG00000134508 | CABLES1 | 2 | 1 | 0 |
| ENSG00000136048 | DRAM1 | 2 | 1 | 0 |
| ENSG00000137959 | IFI44L | 2 | 1 | 0 |
| ENSG00000146072 | TNFRSF21 | 2 | 0 | 0 |
| ENSG00000161642 | ZNF385A | 2 | 0 | 0 |
| ENSG00000162804 | SNED1 | 2 | 2 | 0 |
| ENSG00000168961 | LGALS9 | 2 | 1 | 0 |
| ENSG00000178567 | EPM2AIP1 | 2 | 1 | 0 |
| ENSG00000182150 | C9orf102 | 2 | 0 | 0 |
| ENSG00000244509 | APOBEC3C | 2 | 2 | 0 |
| ENSG00000122547 | EEPD1 | 1 | 0 | 1 |
| ENSG00000198959 | TGM2 | 1 | 0 | 1 |
| ENSG00000259207 | ITGB3 | 1 | 0 | 1 |
| ENSG00000038427 | VCAN | 1 | 0 | 0 |
| ENSG00000068650 | ATP11A | 1 | 2 | 0 |
| ENSG00000076706 | MCAM | 1 | 1 | 0 |
| ENSG00000132872 | SYT4 | 1 | 0 | 0 |
| ENSG00000181192 | DHTKD1 | 1 | 0 | 0 |
| ENSG00000005379 | BZRAP1 | 1 | 1 | 0 |
| ENSG00000011638 | TMEM159 | 1 | 0 | 0 |
| ENSG00000022567 | SLC45A4 | 1 | 0 | 0 |
| ENSG00000047346 | FAM214A | 1 | 0 | 0 |
| ENSG00000069424 | KCNAB2 | 1 | 1 | 0 |
| ENSG00000086730 | LAT2 | 1 | 0 | 0 |
| ENSG00000103316 | CRYM | 1 | 0 | 0 |
| ENSG00000104974 | LILRA1 | 1 | 0 | 0 |
| ENSG00000105246 | EBI3 | 1 | 0 | 0 |
| ENSG00000105339 | DENND3 | 1 | 0 | 0 |
| ENSG00000109654 | TRIM2 | 1 | 1 | 0 |
| ENSG00000111335 | OAS2 | 1 | 0 | 0 |
| ENSG00000111679 | PTPN6 | 1 | 0 | 0 |
| ENSG00000112799 | LY86 | 1 | 0 | 0 |
| ENSG00000113302 | IL12B | 1 | 0 | 0 |
| ENSG00000115155 | OTOF | 1 | 1 | 0 |
| ENSG00000117115 | PADI2 | 1 | 1 | 0 |
| ENSG00000126561 | STAT5A | 1 | 0 | 0 |
| ENSG00000127838 | PNKD | 1 | 1 | 0 |
| ENSG00000129226 | CD68 | 1 | 0 | 0 |
| ENSG00000132622 | HSPA12B | 1 | 0 | 0 |
| ENSG00000133106 | EPSTI1 | 1 | 0 | 0 |
| ENSG00000135245 | HILPDA | 1 | 1 | 0 |
| ENSG00000136634 | IL10 | 1 | 1 | 0 |
| ENSG00000137522 | RNF121 | 1 | 0 | 0 |
| ENSG00000137880 | GCHFR | 1 | 0 | 0 |
| ENSG00000142197 | DOPEY2 | 1 | 0 | 0 |
| ENSG00000144115 | THNSL2 | 1 | 1 | 0 |
| ENSG00000148671 | C10orf116 | 1 | 0 | 0 |
| ENSG00000149289 | ZC3H12C | 1 | 0 | 0 |
| ENSG00000152778 | IFIT5 | 1 | 1 | 0 |
| ENSG00000162772 | ATF3 | 1 | 0 | 0 |
| ENSG00000162849 | KIF26B | 1 | 0 | 0 |
| ENSG00000162896 | PIGR | 1 | 0 | 0 |
| ENSG00000163435 | ELF3 | 1 | 0 | 0 |
| ENSG00000163545 | NUAK2 | 1 | 3 | 0 |
| ENSG00000166145 | SPINT1 | 1 | 0 | 0 |
| ENSG00000168679 | SLC16A4 | 1 | 1 | 0 |
| ENSG00000168952 | STXBP6 | 1 | 0 | 0 |
| ENSG00000170801 | HTRA3 | 1 | 0 | 0 |
| ENSG00000175274 | TP53I11 | 1 | 0 | 0 |
| ENSG00000176058 | TPRN | 1 | 1 | 0 |
| ENSG00000178498 | DTX3 | 1 | 0 | 0 |
| ENSG00000181104 | F2R | 1 | 0 | 0 |
| ENSG00000185885 | IFITM1 | 1 | 0 | 0 |
| ENSG00000197471 | SPN | 1 | 0 | 0 |
| ENSG00000204186 | ZDBF2 | 1 | 0 | 0 |
| ENSG00000256043 | CTSO | 1 | 0 | 0 |
| ENSG00000104381 | GDAP1 | 0 | 1 | 1 |
| ENSG00000148450 | MSRB2 | 0 | 1 | 1 |
| ENSG00000165795 | NDRG2 | 0 | 1 | 1 |
| ENSG00000173511 | VEGFB | 0 | 0 | 1 |
| ENSG00000177082 | WDR73 | 0 | 0 | 0 |
| ENSG00000182319 | PRAGMIN.1 | 0 | 0 | 0 |
| ENSG00000011600 | TYROBP | 0 | 1 | 0 |
| ENSG00000050344 | NFE2L3 | 0 | 0 | 0 |
| ENSG00000050730 | TNIP3 | 0 | 0 | 0 |
| ENSG00000061492 | WNT8A | 0 | 0 | 0 |
| ENSG00000064687 | ABCA7 | 0 | 1 | 0 |
| ENSG00000069956 | MAPK6 | 0 | 0 | 0 |
| ENSG00000070190 | DAPP1 | 0 | 0 | 0 |
| ENSG00000075673 | ATP12A | 0 | 0 | 0 |
| ENSG00000085733 | CTTN | 0 | 0 | 0 |
| ENSG00000089127 | OAS1 | 0 | 0 | 0 |
| ENSG00000089327 | FXYD5 | 0 | 0 | 0 |
| ENSG00000089692 | LAG3 | 0 | 0 | 0 |
| ENSG00000090104 | RGS1 | 0 | 0 | 0 |
| ENSG00000100097 | LGALS1 | 0 | 0 | 0 |
| ENSG00000100239 | PPP6R2 | 0 | 0 | 0 |
| ENSG00000100300 | TSPO | 0 | 0 | 0 |
| ENSG00000100628 | ASB2 | 0 | 1 | 0 |
| ENSG00000102181 | CD99L2 | 0 | 1 | 0 |
| ENSG00000105501 | SIGLEC5 | 0 | 0 | 0 |
| ENSG00000106211 | HSPB1 | 0 | 0 | 0 |
| ENSG00000108469 | RECQL5 | 0 | 0 | 0 |
| ENSG00000109323 | MANBA | 0 | 0 | 0 |
| ENSG00000112116 | IL17F | 0 | 0 | 0 |
| ENSG00000115415 | STAT1 | 0 | 1 | 0 |
| ENSG00000115902 | SLC1A4 | 0 | 2 | 0 |
| ENSG00000116701 | NCF2 | 0 | 0 | 0 |
| ENSG00000116741 | RGS2 | 0 | 0 | 0 |
| ENSG00000117228 | GBP1 | 0 | 0 | 0 |
| ENSG00000118785 | SPP1 | 0 | 0 | 0 |
| ENSG00000119698 | PPP4R4 | 0 | 0 | 0 |
| ENSG00000119801 | YPEL5 | 0 | 1 | 0 |
| ENSG00000120756 | PLS1 | 0 | 0 | 0 |
| ENSG00000120915 | EPHX2 | 0 | 0 | 0 |
| ENSG00000121858 | TNFSF10 | 0 | 0 | 0 |
| ENSG00000123096 | SSPN | 0 | 1 | 0 |
| ENSG00000123146 | CD97 | 0 | 0 | 0 |
| ENSG00000125089 | SH3TC1 | 0 | 0 | 0 |
| ENSG00000126259 | KIRREL2 | 0 | 0 | 0 |
| ENSG00000126353 | CCR7 | 0 | 1 | 0 |
| ENSG00000126709 | IFI6 | 0 | 0 | 0 |
| ENSG00000130589 | RP4-697K14.7.1 | 0 | 0 | 0 |
| ENSG00000131650 | KREMEN2 | 0 | 0 | 0 |
| ENSG00000135114 | OASL | 0 | 0 | 0 |
| ENSG00000135363 | LMO2 | 0 | 0 | 0 |
| ENSG00000136197 | C7orf25 | 0 | 0 | 0 |
| ENSG00000136244 | IL6 | 0 | 0 | 0 |
| ENSG00000137628 | DDX60 | 0 | 0 | 0 |
| ENSG00000138152 | BTBD16 | 0 | 0 | 0 |
| ENSG00000138642 | HERC6 | 0 | 2 | 0 |
| ENSG00000142227 | EMP3 | 0 | 0 | 0 |
| ENSG00000142494 | SLC47A1 | 0 | 0 | 0 |
| ENSG00000143545 | RAB13 | 0 | 0 | 0 |
| ENSG00000143554 | SLC27A3 | 0 | 0 | 0 |
| ENSG00000144218 | AFF3 | 0 | 0 | 0 |
| ENSG00000146094 | DOK3 | 0 | 0 | 0 |
| ENSG00000155008 | APOOL | 0 | 0 | 0 |
| ENSG00000155367 | PPM1J | 0 | 0 | 0 |
| ENSG00000157601 | MX1 | 0 | 1 | 0 |
| ENSG00000157613 | CREB3L1 | 0 | 1 | 0 |
| ENSG00000160190 | SLC37A1 | 0 | 0 | 0 |
| ENSG00000160193 | WDR4 | 0 | 0 | 0 |
| ENSG00000160307 | S100B | 0 | 0 | 0 |
| ENSG00000162419 | GMEB1 | 0 | 0 | 0 |
| ENSG00000165949 | IFI27 | 0 | 0 | 0 |
| ENSG00000166165 | CKB | 0 | 0 | 0 |
| ENSG00000166825 | ANPEP | 0 | 1 | 0 |
| ENSG00000167930 | ITFG3 | 0 | 1 | 0 |
| ENSG00000168298 | HIST1H1E | 0 | 0 | 0 |
| ENSG00000169245 | CXCL10 | 0 | 0 | 0 |
| ENSG00000170054 | SERPINA9 | 0 | 2 | 0 |
| ENSG00000170873 | MTSS1 | 0 | 1 | 0 |
| ENSG00000173890 | GPR160 | 0 | 0 | 0 |
| ENSG00000175793 | SFN | 0 | 0 | 0 |
| ENSG00000175866 | BAIAP2 | 0 | 0 | 0 |
| ENSG00000180879 | SSR4 | 0 | 0 | 0 |
| ENSG00000184348 | HIST1H2AK | 0 | 0 | 0 |
| ENSG00000184588 | PDE4B | 0 | 1 | 0 |
| ENSG00000184979 | USP18 | 0 | 1 | 0 |
| ENSG00000185201 | IFITM2 | 0 | 2 | 0 |
| ENSG00000185291 | IL3RA | 0 | 1 | 0 |
| ENSG00000185507 | IRF7 | 0 | 0 | 0 |
| ENSG00000185745 | IFIT1 | 0 | 0 | 0 |
| ENSG00000187608 | ISG15 | 0 | 0 | 0 |
| ENSG00000188095 | MESP2 | 0 | 0 | 0 |
| ENSG00000188987 | HIST1H4D | 0 | 0 | 0 |
| ENSG00000196154 | S100A4 | 0 | 0 | 0 |
| ENSG00000196226 | HIST1H2BB | 0 | 0 | 0 |
| ENSG00000196374 | HIST1H2BM | 0 | 0 | 0 |
| ENSG00000196433 | ASMT | 0 | 0 | 0 |
| ENSG00000196735 | HLA-DQA1 | 0 | 0 | 0 |
| ENSG00000197355 | UAP1L1 | 0 | 1 | 0 |
| ENSG00000197747 | S100A10 | 0 | 0 | 0 |
| ENSG00000197956 | S100A6 | 0 | 0 | 0 |
| ENSG00000198339 | HIST1H4I | 0 | 0 | 0 |
| ENSG00000198374 | HIST1H2AL | 0 | 0 | 0 |
| ENSG00000198682 | PAPSS2 | 0 | 0 | 0 |
| ENSG00000198734 | F5 | 0 | 0 | 0 |
| ENSG00000203813 | HIST1H3H | 0 | 0 | 0 |
| ENSG00000204388 | HSPA1B | 0 | 0 | 0 |
| ENSG00000204580 | DDR1 | 0 | 0 | 0 |
| ENSG00000213983 | AP1G2 | 0 | 0 | 0 |
| ENSG00000232810 | TNF | 0 | 0 | 0 |
| ENSG00000237541 | HLA-DQA2 | 0 | 0 | 0 |
| ENSG00000256018 | HIST1H3G | 0 | 0 | 0 |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including but not limited to U.S. Patent Application Nos. 61/937,272; 62/010,004; 62/037,497 and 62/273,875, are incorporated herein by reference in their entirety. Aspects of the embodiments can be modified, if necessary, to employ concepts of the various patents, applications and publications to provide further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1-38. (canceled)

39. A method of identifying one or more genes sensitive to inhibition by a MNK inhibitor, the method comprising:
(a) isolating cancer cells from a biological sample from a subject who has cancer;
(b) contacting the isolated cancer cells with the MNK inhibitor;
(c) performing ribosomal profiling to measure translational rate, translational efficiency, mRNA level or any combination thereof of genes present in the cancer cells contacted with the MNK inhibitor; and
(d) identifying one or more genes as sensitive to inhibition by the MNK inhibitor wherein:
(i) the translational rate, translational efficiency, mRNA level or any combination thereof of the one or more genes differs by a loge fold change of at least 0.75 relative to translational rate, translational efficiency, mRNA level or any combination thereof of the same genes in the subject's isolated cancer cells not contacted with a MNK inhibitor; and
(ii) the one or more genes contain a 5′-UTR recognition sequence of Table 8, a 3′-UTR recognition sequence of Table 11, or a combination thereof.

40. The method of claim 39, wherein the one or more identified genes are biomarkers for determining responsiveness to the MNK inhibitor.

41. The method of claim 39, wherein the MNK inhibitor has the following Formula (I): 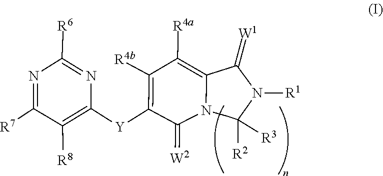
or a stereoisomer, tautomer or pharmaceutically acceptable salt thereof, wherein:
W¹ and W² are independently O, S or N—OR′, where R′ is lower alkyl;
Y is —N(R⁵)—, —O—, —S—, —C(O)—, —S═O, —S(O)₂-, or —CHR⁹—;
R¹ is h_{y}dro_{g}en, lower alkyl, cycloalkyl or heterocyclyl wherein any lower alkyl, cycloalkyl or heterocyclyl is optionally substituted with 1, 2 or 3 J groups;
n is 1, 2 or 3;
R² and R³ are each independently hydrogen, alkyl, alkenyl, alkynyl, aryl, araalkylene, heteroaryl, heteroarylalkylene, cycloalkyl, cycloalkyl alkyl ene, heterocyclyl, or heterocyclylalkylene, wherein any alkyl, aryl, araalkylene, heteroaryl, heteroarylalkylene, cycloalkyl, cycl oal kyl al kyl ene, heterocyclyl, or heterocyclylalkylene, is optionally substituted with 1, 2 or 3 J groups;
or R² and R³ taken together with the carbon atom to which they are attached form a cycloalkyl or heterocyclyl, wherein any cycloalkyl or heterocyclyl is optionally substituted with 1, 2 or 3 J groups;
R^{4a} and R^{4b} are each independently hydrogen, halogen, hydroxyl, thiol, hydroxyalkylene, cyano, alkyl, alkoxy, acyl, thioalkyl, alkenyl, alkynyl, cycloalkyl, aryl, or heterocyclyl;
R⁵ is hydrogen, cyano, or lower alkyl;
or R⁵ and R⁸ taken together with the atoms to which they are attached form a fused heterocyclyl optionally substituted with 1, 2 or 3 J groups;
R⁶, R⁷ and R⁸ are each independently hydrogen, hydroxy, halogen, cyano, amino, alkyl, alkenyl, alkynyl, alkoxy, cycloalkyl, cycloalkylalkylene, cycloalkylalkenylene, alkylaminyl, alkylcarbonylaminyl, cycloalkylcarbonylaminyl, cycloalkyl heterocyclylaminyl, heteroaryl, or heterocyclyl, and wherein any amino, alkyl, alkenyl, alkynyl, alkoxy, cycloalkyl, cycloalkylalkylene, cycloalkylalkenylene, amino, alkyl aminyl, alkylcarbonylaminyl, cycloalkylcarbonylaminyl, cycloalkylaminyl, heterocyclylaminyl, heteroaryl, or heterocyclyl is optionally substituted with 1, 2 or 3 J groups;
or R⁷ and R⁸ taken together with the atoms to which they are attached form a fused heterocyclyl or heteroaryl optionally substituted with 1, 2 or 3 J groups;
J is —SH, —SR⁹, —S(O)R⁹, —S(O)₂R⁹, —S(O)NH₂, —S(O)NR⁹R⁹, —NH₂, —NR⁹R⁹, —COOH, —C(O)OR⁹, —C(O)R⁹, —C(O)—NH₂, —C(O)—NR⁹R⁹, hydroxy, cyano, halogen, acetyl, alkyl, lower alkyl, alkenyl, alkynyl, alkoxy, haloalkyl, thioalkyl, cyanoalkylene, alkylaminyl, NH₂—C(O)-alkylene , NR⁹R⁹—C (O)-alkylen , —CHR⁹—C(O)-lower alkyl, —C(O)-lower alkyl, alkylcarbonylaminyl, cycloalkyl, cycloalkylalkylene, cycloalkylalkenylene, cycloalkylcarbonyl aminyl, cycloalkylaminyl, —CHR⁹—C(O)-cycloalkyl, —C(O)-cycloalkyl, —CHR⁹—C(O)-aryl, —CHR⁹-aryl, —C(O)-aryl, —CHR⁹—C(O)-heterocycloalkyl, —C(O)-heterocycloalkyl, heterocyclylaminyl, or heterocyclyl; or any two J groups bound to the same carbon or hetero atom may be taken together to form oxo; and
R⁹ is hydrogen, lower alkyl or —OH.

42. The method of claim 41, wherein the MNK inhibitor is a compound: 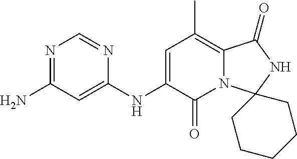 [SMILES: Cc1cc(Nc2cc(N)ncn2)c(=O)n2c1C(=O)NC21CCCCC1]
or a stereoisomer, tautomer or pharmaceutically acceptable salt thereof.

43. The method of claim 39, wherein the translational rate, translational efficiency, mRNA level, or any combination thereof of the one or more genes differs by a loge fold change of 2.0 relative to the translational rate, translational efficiency, mRNA level or any combination thereof of the same genes in the subject's isolated cancer cells not contacted with a MNK inhibitor.

44. The method of claim 39, wherein the translational rate, translational efficiency, mRNA level or any combination thereof of the one or more identified genes is increased relative to the translational rate, translational efficiency, mRNA level or any combination thereof of the same genes in the subject's isolated cancer cells not contacted with a MNK inhibitor.

45. The method of claim 39, wherein the translational rate, translational efficiency, mRNA level or any combination thereof of the one or more identified genes is decreased relative to the translational rate, translational efficiency, mRNA level or any combination thereof of the same genes in the subject's isolated cancer cells not contacted with a MNK inhibitor.

46. The method of claim 39, wherein the cancer cells are contacted with 10 μM of the MNK inhibitor for 3 hours.

47. The method of claim 39, wherein the cancer cells are contacted with 10 μM of the MNK inhibitor for 48 hours.

48. The method of claim 39, wherein the MNK inhibitor is formulated with a pharmaceutically acceptable excipient.

49. The method of claim 39, wherein the cancer is selected from the group consisting of a solid tumor, melanoma, non-small cell lung cancer, renal cell carcinoma, renal cancer, a hematological cancer, prostate cancer, castration-resistant prostate cancer, colon cancer, rectal cancer, gastric cancer, esophageal cancer, bladder cancer, head and neck cancer, thyroid cancer, breast cancer, triple-negative breast cancer, ovarian cancer, cervical cancer, lung cancer, urothelial cancer, pancreatic cancer, glioblastoma, hepatocellular cancer, myeloma, multiple myeloma, leukemia, lymphoma, diffuse large B cell lymphoma (DLBCL), Hodgkin's lymphoma, non-Hodgkin's lymphoma, myelodysplastic syndrome, brain cancer, CNS cancer, malignant glioma, and any combination thereof

50. The method of claim 49, wherein the cancer is lymphoma.

51. The method of claim 39, wherein the subject sample is a tumor tissue sample.

52. The method of claim 39, wherein the subject sample is a hematologic sample.

53. The method of claim 39, wherein the translational rate, translational efficiency, mRNA level, or any combination thereof is of at least two genes in the isolated cancer cells.

54. The method of claim 39, wherein the translational rate, translational efficiency, mRNA level, or any combination thereof is of at least three genes in the isolated cancer cells.

55. The method of claim 39, wherein the translational rate, translational efficiency, mRNA level, or any combination thereof is of at least four genes in the isolated cancer cells.

56. The method of claim 39, wherein the one or more identified genes are selected from the group consisting of NR2F1, VLDLR, C2CD2L, BCL9L, CAV2, ACCN2, FZD5, RBKS, ULK2, KLF5, KLF9, SYT4, TMSB4Y, SKI, CENPBD1, LPAR5, ST3GAL1, WNT8A, WASF1, B3GNT7, TNFRSF14, VANGL2, ZNF771, RPS6KL1, ZNF425, CCDC85C, PER3, RASGRF1, EDN1, FLT3LG, SLC35A2, NR4A3, GLIPR2, ARMC7, PPP1R3D, PSRC1, KIAA0748, SETD1B, SLC16A3, MOB3C, LHFPL2, TTLL11, PCDH9, STMN3, FAM212B, C6orf225, SMN2 and any combination thereof.

57. The method of claim 39, wherein the ribosomal profiling is performed to measure the translational rate of genes present in the isolated cancer cells.

58. The method of claim 39, wherein the ribosomal profiling is performed to measure the translational efficiency of genes present in the isolated cancer cells.
